(19) United States
(12) Patent Application Publication
(10) Pub. No.: US 2025/0381270 A1
(43) Pub. Date: Dec. 18, 2025

(54) ISOLATING ACTIVE T-CELL CARS FROM BULK TRANSDUCED HUMAN T-CELLS

(71) Applicant: The Regents of the University of California, Oakland, CA (US)

(72) Inventors: Charles PRUSSAK, San Diego, CA (US); Christopher OH, Porter Ranch, CA (US)

(21) Appl. No.: 18/854,408

(22) PCT Filed: Apr. 6, 2023
(86) PCT No.: PCT/US2023/065478

§ 371 Date: Oct. 4, 2024

Related U.S. Application Data
(60) Provisional application No. 63/328,215, filed on Apr. 6, 2022.

(51) Int. Cl. A61K 40/11; A61K 35/17; A61K 40/31; A61K 40/42; C07K 16/00; C12N 5/00; C12N 5/0783

(52) U.S. Cl. CPC A61K 40/11; A61K 35/17; A61K 40/31; A61K 40/4202; C07K 16/00; C12N 5/0087; C12N 5/0636; C07K 2317/52

(57) ABSTRACT

Provided herein, inter alia, are methods for the isolation and purification of CAR T-Cells from bulk transduced human T-cells. Also provided herein are chimeric peptide-CAR T-cell complexes useful for the isolation of CAR T-cells. In addition, pharmaceutical compositions and methods for treating cancer are described herein.

### CROSS-REFERENCE TO RELATED APPLICATION

This application claims the benefit of priority under 35 U.S.C. § 119 (e) of the U.S. Patent Application No. 63/328,215, filed Apr. 6, 2022, which is hereby incorporated by reference in its entirety and for all purposes.

### REFERENCE TO A SEQUENCE LISTING

The material in the accompanying Sequence Listing is hereby incorporated by reference in its entirety. The accompanying file named “048537-643001WO_SL_ST26.xml” was created on Apr. 3, 2023 and is 5,602 bytes.

### BACKGROUND

The current art for generating purified Chimeric Antigen Receptors (CARs) for the most part requires the employment of cis-acting elements that are expressed by the lentiviral expression vector or are an integral part of a T-lymphocyte. It is common in the art that a non-signaling portion of the Epidermal Growth Factor (EGF) receptor is expressed as part of a lentiviral CAR expression product. When the lentivirus expresses the CAR product, the inert EGF receptor domain is also expressed on the surface of the transduced cells and can thereby be used for isolation and detection purposes of the CAR T-cell (Wang et al., Blood, 2011; 118 (5): 1255-1263). Provided herein are methods and compositions addressing needs in the art of isolating and purifying CAR T-cells for subsequent therapeutic use.

### BRIEF SUMMARY

In an aspect is provided a method of isolating a CAR T-cell, wherein the method includes: (i) contacting a cell population including a CAR-expressing T-cell with a labeled CAR-binding peptide thereby forming a labeled CAR T-cell-peptide complex; (ii) contacting the labeled CAR T-cell-peptide complex with a label-binding magnetic particle thereby forming a bound labeled CAR T-cell-peptide complex; and (iii) separating said bound labeled CAR T-cell-peptide complex from the cell population, thereby isolating a CAR T-cell.

In another aspect is provided a method of isolating a chimeric antigen receptor (CAR)-expressing T-cell, the method including: (i) contacting a cell population including a CAR-expressing T-cell with a chimeric binding peptide including a CAR-binding domain, a ligand binding domain and a detectable moiety, thereby forming a chimeric peptide-CAR T-cell complex; (ii) contacting the chimeric peptide-CAR T-cell complex with a ligand, thereby forming a ligand-bound chimeric peptide-CAR T-cell complex; and (iii) separating the ligand-bound chimeric peptide-CAR T-cell complex from the cell population, thereby isolating a CAR T-cell population.

In another aspect is provided a method of isolating a chimeric antigen receptor (CAR)-expressing T-cell, the method including: (i) contacting a cell population including a CAR-expressing T-cell with a chimeric binding peptide including a CAR-binding domain and a ligand binding domain, thereby forming a chimeric peptide-CAR T-cell complex; (ii) contacting the chimeric peptide-CAR T-cell complex with a ligand, thereby forming a ligand-bound chimeric peptide-CAR T-cell complex; and (iii) separating the ligand-bound chimeric peptide-CAR T-cell complex from the cell population, thereby isolating a CAR T-cell population.

In another aspect is provided a chimeric peptide-chimeric antigen receptor (CAR) T-cell complex including: a CAR-expressing T-cell bound to a chimeric binding peptide, the chimeric binding peptide including a CAR-binding domain a ligand binding domain, and a detectable moiety.

In another aspect is provided a pharmaceutical composition including an therapeutically effective amount of the purified CAR T-cell population provided herein including embodiments thereof and a pharmaceutically acceptable excipient.

In another aspect is provided a method of treating cancer in a subject in need thereof including administering to the subject a therapeutically effective amount of a purified CAR T-cell provided herein including embodiments thereof or a pharmaceutical composition provided herein including embodiments thereof, thereby treating the cancer.

### BRIEF DESCRIPTION OF THE DRAWINGS

FIGS. 1A-1I illustrate the generation and purification of the T-cell CAR. These figures show collection of human leukapheresed lymphocytes, which were purified, activated and transduced with a ROR1 CAR lentiviral expression vector. Following transduction, the generation of the anti-ROR1 CAR expression product was analyzed by flow cytometry employing fluorescently labeled anti-ROR1-Ig for detection. As shown in FIG. 1F, the CAR product (second peak) was detected on Day 17 and the ROR1 CAR^{POS} population will continue to expand for up to 60 days and may become the major constituent of the expanding T-cell population. In FIGS. 1I, the T-cell CAR product was purified by affinity purification process from Day 17 (FIG. 1F) bulk culture by using ROR1-Ig bound magnetic beads. Employing the affinity purification process, >50% of the CAR product may be achieved routinely in the bulk lymphocyte cultures.

FIG. 2 illustrates isolation of purified CAR T-cells by using a labeled antigen binding fusion protein.

FIGS. 3A-3E show the expression of two types of chimeric antigen receptor (CAR): anti-ROR2 CAR (6E6 and 4G9) and anti-ROR1 CAR (UC961). Control T-cells showed some background level expression (7.2%) of ROR2 CAR (FIG. 3A). The 4G9 (FIG. 3B) and 6E6 (FIG. 3C) ROR2 CAR T-cells purified using the method described herein showed 56.6% and 61.1% CAR expression, respectively, as determined by flow cytometry. In contrast, the control for the ROR1 CAR showed no CAR expression (FIG. 3D), while the UC961 anti-ROR1 CAR T-cells purified using the method described herein showed a high expression of 90.6% (FIG. 3E).

FIGS. 4A-4B show data from the Chromium Release assay performed on the CAR T-cells from FIG. 3. The purified anti-ROR1 UC961 CAR T-cells exhibited high specificity against MEC1-ROR1 cells while showing minimal activity against MEC1-ROR2 cells (FIG. 4A). In contrast, the anti-ROR2 (4G9 and 6E6) CAR T-cells displayed very specific activity against MEC1-ROR2 cells while having minimal activity against MEC1-ROR1 cells (FIG. 4B). The control T-cells (AO) demonstrated background levels of activity due to non-specific T-cell effectors.

FIGS. 5A-5D show that the purification method generates a population of CAR T-cells with increased CAR expression. Control cells demonstrated background levels of expression as determined by flow cytometry (FIG. 5B). FIGS. 5C-5D show purified anti-ROR2 CAR (6E6 and 4G9) had a more uniformly high expression of the CAR. After purification, 6E6 CAR T-cells had 98% expression (FIG. 5C) and 4G9 CAR T-cells had 86.7% expression (FIG. 5D). Purified anti-ROR1 CAR T-cells (UC961) had 89.3% expression of the CAR (FIG. 5E).

FIGS. 6A-6C show a comparison of the CAR expression of purified CAR T-cells (UC961+) and non-purified CAR T-cells (UC961). FIG. 6B shows the CAR expression of control cells (AO) and anti-ROR1 CAR T-cells (UC961) at day 12 (D12) of culture. FIG. 6C shows that by day 14 (D14), the non-purified CARs (UC961; middle panel) showed a decline in CAR expression to 22.2%, while the purified UC961+CAR T-cells showed a significant increase in CAR expression, progressing to 63% (bottom panel).

FIGS. 7A-7E show CAR expression of non-purified CAR (UC961) and control (AO) T-cells between day 2 (D2) and day 19 (D19). The non-purified UC961 CAR T-cells showed a progressive increase in CAR expression, reaching up to 16.2% after 19 days of culture, compared to untransduced control T-cells.

FIGS. 8A-8F show CAR expression of positively selected, purified (UC961+) and non-purified (UC961) CAR T-cells. The purification of UC961 CAR T-cells was performed on day 6 (D6). On day 7 (D7), CAR expression of the purified UC961+CAR T-cells was analyzed, revealing a 37.4% enrichment of the highly positive population of UC961 CARs (FIG. 8C). On day 9 (D9), the first comparison of the purified UC961+CAR T-cells showed a significant increase in CAR expression, with 50.1% expression observed (FIG. 8D, bottom panel), compared to only 15% from the non-purified UC961 CAR T-cells (FIG. 8D, top panel). The enrichment of cells with high CAR expression in the purified CAR T-cells continued to increase on days 12 (D12; FIG. 8E) and 19 (D19; FIG. 8F) compared to non-purified CAR T-cells.

FIGS. 9A-9B show cytotoxic activity of purified CAR T-cells (UC961+) compared to non-purified CAR T-cells (UC961) over time. Results showed that selected CAR T-cells exhibited much higher activity compared to unselected CAR T-cells. It is worth noting that both unselected and selected CAR T-cells showed significant activity compared to non-transduced control T-cells.

FIGS. 10A-10F show cytotoxic activity of purified CAR T-cells (UC961+) versus non-purified CAR T-cells (UC961) using celltracker measured by Incucyte over time. MEC1-ROR1 (M1R1) or MEC1 (M1) cells were used as target cells with the following effector cells: control (AO) T-cells, non-purified UC961 CAR T-cells, and purified UC961+CAR T-cells. The results demonstrate that purified UC961+CAR T-cells had much higher and more specific cytotoxic activity compared to non-purified UC961 CAR T-cells. Furthermore, both non-purified and purified CAR T-cells showed more cytotoxic activity than the control (AO) untransduced T-cells.

FIGS. 11A-11E show CAR expression in transduced and purified Jurkat cell lines, including Wildtype (WT) Jurkats (control), Jurkat-UC961, Jurkat-6E6, purified Jurkat-UC961+, and purified Jurkat-6E6+. The WT Jurkat cells showed no CAR expression (FIG. 11A). Jurkat-UC961 displayed a 65.4% CAR expression, with a smaller population of highly positive Jurkat CARs (FIG. 11B). Jurkat-6E6 showed 31.1% CAR expression, also with a smaller population of highly positive Jurkat CARs (FIG. 11C). In the respective purified Jurkat-UC961+ (FIG. 11D) and Jurkat-6E6+ T-cells (FIG. 11E), the CAR expression was mostly from the highly positive Jurkat CAR population, with an overall expression of 99.4%.

FIGS. 12A-12B show CAR expression in Jurkat cells compared using flow cytometry. FIG. 12A shows the expression of Wildtype (WT) Jurkats compared with transduced Jurkat CARs, namely Jurkat-UC961 (left panel) and Jurkat-6E6 (right panel). FIG. 12B shows the CAR expression of transduced Jurkat cells compared with the purified Jurkat CARs, namely Jurkat-UC961+ (left panel) and Jurkat-6E6+ (right panel).

FIG. 13 shows ROR2 expression of MEC1 cells using flow cytometry. The flow cytometry analysis of the ROR2-expression vector-transduced, magnetically-purified MEC1-ROR2 cell line reveals a high level of ROR2 expression, with 98.7% of the cells expressing this protein. This indicates that the MEC1-ROR2 cell population is essentially pure for ROR2 expression, as demonstrated in the flow diagram.

FIG. 14 shows data from a flow cytometry analysis depicting a magnetic-bead purified MEC1-ROR1 cell line originally transduced with a ROR1-expression vector (top three panels). Additionally, flow cytometry analysis illustrating primary chronic lymphocytic leukemia (CLL) cells that naturally express ROR1 for comparison purposes (bottom three panels).

FIGS. 15A-15B shows data from a flow cytometry analysis of the magnetically-purified MEC1-ROR1/ROR2 target cells and indicates high levels of expression for both ROR1 and ROR2, with ROR1 expression at 97.3% (FIG. 15A) and ROR2 expression at 98.1% (FIG. 15B).

FIGS. 16A-16B show in vitro cell activity of ROR1 CAR T-cells in a 4-hour chromium release assay (FIG. 16A) and a 120-hour ACEA impedance assay (FIG. 16B). ROR1 CAR T-cell products were generated from 2 healthy donors and tested in a chromium release assay at the indicated E:T ratios against leukemia Mec1 ROR^{pos} cells in FIG. 16A and an ACEA impedance assay against MB 231 ROR1^{pos} breast cancer cells in FIG. 16B. The anti-ROR1 CAR T-cells demonstrated high and specific cytotoxicity without significant killing of ROR1-negative target cells.

FIGS. 17A-17B show bioluminescence imaging of mice inoculated with MEC1-ROR1 cells and with ROR1 CAR T-cells. Animals treated with CAR T-cells had reduced disease burden compared to controls. Mice treated with 3e6 CAR-T reduced the leukemic burden to background levels by day 30 and controlled disease for the remainder of the study. Animals in the control groups (untreated, mock transduced) had to be sacrificed on day 20. The CAR-T treated cohort had only minimal amounts of disease at the end of the study. FIG. 17B shows the total bioluminescent product collected from the mice. Squares and circles are controls; triangles are ROR1 CAR T-cell-treated.

FIGS. 18A-18B show anti-ROR1 CAR T-cells specifically killed PDX PCSD13 cells in Effector:Target (E:T) dose-dependent manner in culture in Incucyte Cytotoxicity assay. FIG. 18A shows the cytotoxicity data from control T-cells plus PCSD13 (Target) cells freshly isolated from xenograft and cultured for 48 hours. FIG. 18B shows cytotoxicity data from ROR1 CAR T-cells (effectors) cultured with PCSD13 (Target) cells. The highest dose of anti-ROR1 CAR T-cells completely killed the PCSD13 cells (E:T 3) compared to no CAR T-cells (E:T 0).

FIG. 19 shows specific lysis of primary CLL cells by patient-derived ROR1 CAR T-cells. Primary cells were collected from two patients with CLL by leukapheresis. The bulk cells were purified over a Ficoll gradient and an aliquot of the cells was collected and frozen in bulk. A separate aliquot of freshly purified cells was further processed, total CD3⁺ cells were collected and ROR1 CAR T-cells were generated. For analysis, frozen bulk PBMC (>80% CD19⁺ CLL cells) were thawed, washed and labeled with ⁵¹Chromium. CAR T-cells were added to the patient CLL cells for a 4-hour chromium release assay at 3 different effector-to-target (ET) ratios. Results are from the average of 3 replicates and error bars are for standard error of the mean. Patient 1=71 years old; Patient 2=79 years old.

FIG. 20 shows data from a flow cytometry analysis. After the initial transduction of Jurkat cells with the UC961 CAR expression vector, flow cytometry analysis was performed to determine the baseline expression level of the CAR. The unpurified (unselected) Jurkat-UC961 cells showed 21.8% CAR expression. To further enrich the CAR-expressing population, magnetic bead purification or selection was performed. The Jurkat-UC961+ cells that were purified (selected) by magnetic beads were subsequently analyzed by flow cytometry. The analysis showed that the CAR expression of the Jurkat-UC961+ cells was significantly enriched to 90.6%, demonstrating the success of the magnetic bead selection method in isolating CAR-expressing cells.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### I. DEFINITIONS

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The abbreviations used herein have their conventional meaning within the chemical and biological arts. The chemical structures and formulae set forth herein are constructed according to the standard rules of chemical valency known in the chemical arts.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. The detectable moiety may be used to label biomolecules. Thus, in embodiments, the detectable moiety is a label.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), Van der Waal's bond(s)/interactions, hydrogen bond(s), polar bond(s), or combinations or mixtures thereof).

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The term “bispecific T-cell engager (BiTE)”, “BiTe” or “bispecific antibody” as provided herein is used according to its conventional meaning well known in the art and refers to a bispecific recombinant protein capable to simultaneously bind to two different antigens. In contrast to traditional monoclonal antibodies, BiTE antibodies consist of two independently different antibody regions (e.g., two single-chain variable fragments (scFv)), each of which binds a different antigen. One antibody region engages effector cells (e.g., T-cells) by binding an effector cell-specific antigen (e.g., CD3 molecule) and the second antibody region binds a target cell (e.g., cancer cell or autoimmune-reactive cell) through a cell surface antigen (e.g., BAFF-R) expressed by said target cell. Binding of the BiTE to the two antigens will link the effector cell (e.g., T-cell) to the target cell (e.g., tumor cell) and activate the effector cell (e.g., T-cell) via effector cell-specific antigen signaling (e.g., CD3 signaling). The activated effector cell (e.g., T-cell) will then exert cytotoxic activity against the target cell (e.g., tumor cells).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

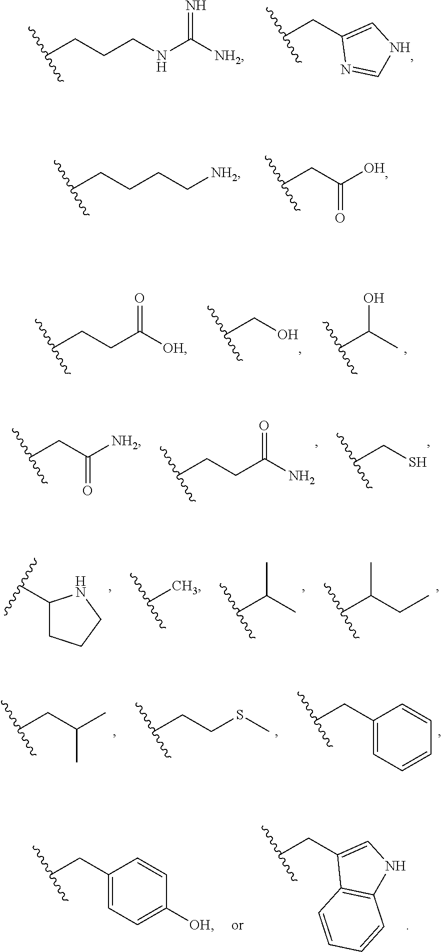

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino) octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe (2-Br)—OH, Boc-Phe (4-Br)—OH, Boc-D-Phe (4-Br)—OH, Boc-D-Phe (3-Cl)—OH, Boc-Phe (4-NH2)-OH, Boc-Phe (3-NO2)-OH, Boc-Phe (3,5-F2)-OH, 2-(4-Boc-piperazino)-2-(3,4-dimethoxyphenyl) acetic acid purum, 2-(4-Boc-piperazino)-2-(2-fluorophenyl) acetic acid purum, 2-(4-Boc-piperazino)-2-(3-fluorophenyl) acetic acid purum, 2-(4-Boc-piperazino)-2-(4-fluorophenyl) acetic acid purum, 2-(4-Boc-piperazino)-2-(4-methoxyphenyl) acetic acid purum, 2-(4-Boc-piperazino)-2-phenylacetic acid purum, 2-(4-Boc-piperazino)-2-(3-pyridyl) acetic acid purum, 2-(4-Boc-piperazino)-2-[4-(trifluoromethyl)phenyl]acetic acid purum, Boc-β-(2-quinolyl)-Ala-OH, N—Boc-1,2,3,6-tetrahydro-2-pyridinecarboxylic acid, Boc-B-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen (Trt)-OH, Fmoc-Phe (2-Br)—OH, Fmoc-Phe (4-Br)—OH, Fmoc-Phe (3,5-F2)-OH, Fmoc-β-(4-thiazolyl)-Ala-OH, Fmoc-β-(2-thienyl)-Ala-OH, 4-(Hydroxymethyl)-D-phenylalanine.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region; also referred to herein as “Fc domain”) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. In embodiments, the Fc region includes a constant heavy chain domain 3 (CH3 domain) and a constant heavy chain domain 2 (CH2 domain).

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

By “cell culture” or “culture” is meant the maintenance of the cells in an artificial, in vitro environment. The term “cell culture” also encompasses cultivating individual cells and tissues. The cells being cultured according to the present invention, whether primary or not, can be cultured and plated or suspended according to the disclosed conditions. The examples herein demonstrate at least one functional set of culture conditions that can be used in conjunction with the methods described herein. If not known, plating or suspension and culture conditions for a given animal or human cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be precoated with a natural, recombinant or synthetic attachment factor or factors or peptide fragments thereof, such as but not limited to collagen, fibronectin and natural or synthetic fragments thereof.

As used herein, the term “cell culture medium” is used in accordance with its plain and ordinary meaning and encompasses any gel or liquid created to support cellular growth in an artificial environment. A culture medium plays an integral role in cell culture technology, supporting in vitro cellular research. It is the medium that supplies the nutrients necessary for cell cultures to survive and proliferate. The cell culture medium also provides the correct osmolality and pH. There are a variety of different types of cell culture media that accommodate cells from mammals, plants, insects, bacteria, yeast, viruses, and more. In embodiments, the cell culture medium includes one or more growth factors. The term cell culture medium may be used interchangeably with cell medium or culture medium.

As used herein, the term “growth factor” is used in accordance with its plain and ordinary meaning and encompasses a biomolecule capable of supporting, stimulating, or promoting cell growth. In embodiments, the growth factor is a diffusible signaling molecule. In embodiments, the growth factor is a protein. In embodiments, the growth factor is a cytokine. In embodiments, the growth factor is a steroid hormone. In embodiments, the growth factor stimulates cell growth, differentiation, survival, or inflammation. In embodiments, the growth factor is secreted. In embodiments, the growth factor is secreted by neighboring cells or distant cells. In embodiments, the growth factor is exogenously applied.

As used herein, the term “culturing T-cells” is used in accordance with its plain and ordinary meaning and refers to the process by which cells are grown under controlled conditions, generally outside their natural environment. After the cells of interest, herein T-cells, have been isolated from a donor or patient blood products, they can subsequently be maintained under carefully controlled conditions. These conditions vary for each cell type, but generally consist of a suitable vessel with a substrate or medium that supplies the essential nutrients.

As used herein, the term “donor” is used in accordance with its plain and ordinary meaning and refers to a subject that supplies living tissue or blood to be used in another body (e.g., a person who furnished blood for transfusion in a histo-compatible recipient). In embodiments, the donor is a living human donor. In embodiments, the donor is a healthy living human donor. In embodiments, the donor is a living human donor with cancer.

As used herein, the term “isolating T-cells” is used in accordance with its plain and ordinary meaning and refers to the process by which interested cells, herein T-cells, can be isolated from a larger population of cells. In embodiments, the isolation of T-cells includes isolating peripheral blood mononuclear cells (PBMCs) from donor human leukapheresed samples. In embodiments, the isolation of T-cells includes isolating CD3-positive (CD3^{pos} or CD3⁺) T-cells from PBMCs. In embodiments, the isolation of T-cells includes isolating a population of CAR-expressing T-cells from T-cells that do not have CAR expression.

As used herein, the term “expanding CAR T-cells” is used in accordance with its plain and ordinary meaning and refers to the process by which CAR-expressing T-cells are grown under controlled conditions in a cell culture to increase the number of CAR-expressing T-cells. In embodiments, an isolated CAR T-cells population is expanded to increase the numbers of CAR T-cells.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a cell (e.g., T-cell)-ligand (e.g. CD3, CD28, etc.) interaction means positively affecting (e.g. increasing) the activity or function of the cell relative to the activity or function of the cell in the absence of the ligand. In aspects activation means positively affecting (e.g. increasing) the proliferation rate or biologic activity of the cell relative to the rate or activity of the cell in the absence of the activator. The terms may reference activation, or activating, sensitizing, or up-regulating signal transduction or enzymatic activity or gene expression of a cell. Thus, activation may include, at least in part, partially or totally increasing stimulation, increasing or enabling activation, or activating, sensitizing, or up-regulating signal transduction or enzymatic activity or gene expression relative to the absence of the activator. Activation may include, at least in part, partially or totally increasing stimulation, increasing or enabling activation, or activating, sensitizing, or up-regulating signal transduction or enzymatic activity or gene expression.

As used herein, the term “activated T-cell” is used in accordance with its plain ordinary meaning and refers to a mature T-cell that has been contacted by a biomolecule and stimulated to express antigen-specific T-cell receptors on its surface. In embodiments, activated T-cells express an antigen specific T-cell receptor that recognizes, interacts with, or binds its antigen. In embodiments, the antigen interaction with the antigen-specific T-cell receptor stimulates the activated T-cell to respond to the presence of the antigen. In embodiments, the activated T-cell respond by entering the cell cycle, secreting cytokines or lytic enzymes, or initiating the cell-based functions of the immune system. In embodiments, a T-cell is activated by contacting the cell with CD3 and/or CD28.

The term “purified CAR T-cell population” is used herein according to its plain ordinary meaning and refers to a population of CAR-expressing T-cells which have been positively selected for expression of a preferred characteristic. In embodiments, the preferred characteristic is expression of a CAR of interest. In embodiments, the selection occurs by isolating the population of CAR-expressing T-cells from a cell population that includes cells which do not express a CAR at a detectable level. In embodiments, the purified CAR T-cell population does not include cells that do not express detectable levels of a CAR.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components

A “ROR1 protein” as referred to herein includes any of the recombinant or naturally-occurring forms of the tyrosine-protein kinase transmembrane receptor (ROR1) also known as neurotrophic tyrosine kinase receptor-related 1 (NTRKR1) or variants or homologs thereof that maintain ROR1 kinase activity (e.g. within at least 50%, 80%, 90%, 95%, 96%, 97%, 98%, 99% or 100% activity compared to ROR1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ROR1 protein. In embodiments, the ROR1 protein is substantially identical to the protein identified by the UniProt reference number Q01973 or a variant or homolog having substantial identity thereto. In embodiments, the ROR1 protein is substantially identical to the protein identified by the UniProt reference number Q9Z139 or a variant or homolog having substantial identity thereto.

A “ROR2 protein” as referred to herein includes any of the recombinant or naturally-occurring forms of the tyrosine-protein kinase transmembrane receptor (ROR2) also known as neurotrophic tyrosine kinase receptor-related 1 (NTRKR2) or variants or homologs thereof that maintain ROR2 kinase activity (e.g. within at least 50%, 80%, 90%, 95%, 96%, 97%, 98%, 99% or 100% activity compared to ROR2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ROR2 protein. In embodiments, the ROR2 protein is substantially identical to the protein identified by the UniProt reference number Q01974 or a variant or homolog having substantial identity thereto. In embodiments, the ROR2 protein is substantially identical to the protein identified by the UniProt reference number Q9Z138 or a variant or homolog having substantial identity thereto.

A “CD19 protein” as referred to herein includes any of the recombinant or naturally-occurring forms of the cluster of differentiation 19 molecule (CD19) also known as B-lymphocyte antigen CD19, B-lymphocyte surface antigen B4, T-cell surface antigen Leu-12, CVID3, or variants or homologs thereof that maintain CD19 activity (e.g. within at least 50%, 80%, 90%, 95%, 96%, 97%, 98%, 99% or 100% activity compared to CD19). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 protein. In embodiments, the ROR2 protein is substantially identical to the protein identified by the UniProt reference number P15391 or a variant or homolog having substantial identity thereto. In embodiments, the ROR2 protein is substantially identical to the protein identified by the UniProt reference number P25918 or a variant or homolog having substantial identity thereto.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example, certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. EMBODIMENTS

### Methods of Isolating CAR T-Cells

Provided herein are, inter alia, methods for isolating and using purified CAR T-cell population. Applicant has discovered that CAR-expressing T-cells can be isolated and purified using a chimeric binding protein that directly targets the CAR of interest. The methods provided herein do not rely on expression of a second recombinant protein to effectuate the isolation. The isolated CAR T-cell population generated using the methods described herein including embodiments thereof highly homogenous and express high levels of the CAR of interest.

For the methods provided herein human T-cells were isolated from peripheral blood mononuclear cells (PBMCs) from the blood of patients (e.g., B cell CLL patients) or healthy individuals using standard cell separation methods (e.g., CD4, CD8, and/or CD3-coated microbeads). Isolated T-cells may then be activated using standard cell culture procedures, expanded for about 1-3 days and subsequently transduced with a viral vector (e.g., lentiviral vector) expressing the CAR of interest. The CAR-transduced T-cell population may then be expanded in appropriate tissue culture media for about 7 to 28 days. The isolated, transduced and expanded T-cell population may then be used in the methods provided herein to select for a highly purified and active CAR T-cell population. Thus, in embodiments, the cell population of step (i) is an isolated T-cell population. In embodiments, the isolated T-cell population is an activated T-cell population. In embodiments, the isolated T-cell population is an activated and transduced T-cell population. In embodiments, the isolated T-cell population expresses CD4, CD8, or CD3 or any combination thereof. In embodiments, the isolated T-cell population expresses CD4. In embodiments, the isolated T-cell population expresses CD8. In embodiments, the isolated T-cell population expresses CD3. The isolated T-cell population may be activated with CD3 and/or CD28-expressing microbeads. The isolated T-cell population may be activated with CD3 and CD28-expressing microbeads. In embodiments, the isolated T-cell population may be activated with CD3 or CD28-expressing microbeads. In embodiments, the isolated T-cell population may be transduced with a viral vector (e.g. lentiviral vector) to generate a CAR-expressing T-cell population. In embodiments, the lentiviral vector encodes a CAR that targets a cancer antigen. In embodiments, the isolated T-cell population is transduced with a lentiviral vector. In embodiments, the lentiviral vector encodes a scFv that targets a cancer antigen. In further embodiments, the transduction step may include a transducing reagent. In embodiments, the transducing reagent is Synperonic F108. In embodiments, the transducing reagent is Lentiboost. In embodiments, the transducing reagent is protamine sulfate. In embodiments, the transducing reagent is polybrene.

Thus, in an aspect is provided a method of isolating a CAR T-cell, wherein the method includes: (i) contacting a cell population including a CAR-expressing T-cell with a labeled CAR-binding peptide thereby forming a labeled CAR T-cell-peptide complex; (ii) contacting the labeled CAR T-cell-peptide complex with a label-binding magnetic particle thereby forming a bound labeled CAR T-cell-peptide complex; and (iii) separating said bound labeled CAR T-cell-peptide complex from the cell population, thereby isolating a CAR T-cell.

In embodiments, the bound labeled CAR T-cell peptide complex is isolated from the cell population with a magnet.

In embodiments, the CAR-T-cell is isolated from the labeled CAR-binding peptide and the label-binding magnetic particle, to obtain a purified CAR-T-cell.

In another aspect is provided a method of isolating a chimeric antigen receptor (CAR)-expressing T-cell, the method including: (i) contacting a cell population including a CAR-expressing T-cell with a chimeric binding peptide including a CAR-binding domain, a ligand binding domain and a detectable moiety, thereby forming a chimeric peptide-CAR T-cell complex; (ii) contacting the chimeric peptide-CAR T-cell complex with a ligand, thereby forming a ligand-bound chimeric peptide-CAR T-cell complex; and (iii) separating the ligand-bound chimeric peptide-CAR T-cell complex from the cell population, thereby isolating a CAR T-cell population.

In another aspect is provided a method of isolating a chimeric antigen receptor (CAR)-expressing T-cell, the method including: (i) contacting a cell population including a CAR-expressing T-cell with a chimeric binding peptide including a CAR-binding domain and a ligand binding domain, thereby forming a chimeric peptide-CAR T-cell complex; (ii) contacting the chimeric peptide-CAR T-cell complex with a ligand, thereby forming a ligand-bound chimeric peptide-CAR T-cell complex; and (iii) separating the ligand-bound chimeric peptide-CAR T-cell complex from the cell population, thereby isolating a CAR T-cell population.

In embodiments, the method further includes after the separating a step (iv) of expanding the isolated CAR T-cell population to form a purified CAR T-cell population. In embodiments, the purified CAR T-cell population does not include the chimeric binding peptide. In embodiments, the chimeric binding peptide is removed from the CAR T-cell. In embodiments, the chimeric binding peptide is separated from the CAR T-cell by enzymatic, physical or chemical means. In embodiments, the chimeric binding peptide is separated from the CAR T-cell by enzymatic means. In embodiments, the chimeric binding peptide is detached from the CAR T-cell by physical means. In embodiments, the chimeric binding peptide is separated from the CAR T-cell by chemical means. In embodiments, the chimeric binding peptide is separated from the CAR T-cell using commercially available releasing reagents. In embodiments, the releasing reagents include enzymes and non-enzymatic alternatives that break bonds between two molecules, thereby forming two separate, unbound molecules. In embodiments, the releasing reagents separate two molecules.

In embodiments, the isolated CAR T-cell population is expanded for about 3 days to about 60 days. In embodiments, the isolated CAR T-cell population is expanded for about 4 days to about 60 days. In embodiments, the isolated CAR T-cell population is expanded for about 5 days to about 60 days. In embodiments, the isolated CAR T-cell population is expanded for about 6 days to about 60 days. In embodiments, the isolated CAR T-cell population is expanded for about 7 days to about 60 days. In embodiments, the isolated CAR T-cell population is expanded for about 8 days to about 60 days. In embodiments, the isolated CAR T-cell population is expanded for about 9 days to about 60 days. In embodiments, the isolated CAR T-cell population is expanded for about 10 days to about 60 days. In embodiments, the isolated CAR T-cell population is expanded for about 11 days to about 60 days. In embodiments, the isolated CAR T-cell population is expanded for about 12 days to about 60 days. In embodiments, the isolated CAR T-cell population is expanded for about 13 days to about 60 days. In embodiments, the isolated CAR T-cell population is expanded for about 14 days to about 60 days. In embodiments, the isolated CAR T-cell population is expanded for about 15 days to about 60 days. In embodiments, the isolated CAR T-cell population is expanded for about 16 days to about 60 days. In embodiments, the isolated CAR T-cell population is expanded for about 17 days to about 60 days. In embodiments, the isolated CAR T-cell population is expanded for about 18 days to about 60 days. In embodiments, the isolated CAR T-cell population is expanded for about 19 days to about 60 days. In embodiments, the isolated CAR T-cell population is expanded for about 20 days to about 60 days. In embodiments, the isolated CAR T-cell population is expanded for about 25 days to about 60 days. In embodiments, the isolated CAR T-cell population is expanded for about 30 days to about 60 days. In embodiments, the isolated CAR T-cell population is expanded for about 35 days to about 60 days. In embodiments, the isolated CAR T-cell population is expanded for about 40 days to about 60 days. In embodiments, the isolated CAR T-cell population is expanded for about 45 days to about 60 days. In embodiments, the isolated CAR T-cell population is expanded for about 50 days to about 60 days. In embodiments, the isolated CAR T-cell population is expanded for about 55 days to about 60 days.

In embodiments, the isolated CAR T-cell population is expanded for about 3 days to about 55 days. In embodiments, the isolated CAR T-cell population is expanded for about 3 days to about 50 days. In embodiments, the isolated CAR T-cell population is expanded for about 3 days to about 45 days. In embodiments, the isolated CAR T-cell population is expanded for about 3 days to about 40 days. In embodiments, the isolated CAR T-cell population is expanded for about 3 days to about 35 days. In embodiments, the isolated CAR T-cell population is expanded for about 3 days to about 30 days. In embodiments, the isolated CAR T-cell population is expanded for about 3 days to about 25 days. In embodiments, the isolated CAR T-cell population is expanded for about 3 days to about 20 days. In embodiments, the isolated CAR T-cell population is expanded for about 3 days to about 19 days. In embodiments, the isolated CAR T-cell population is expanded for about 3 days to about 18 days. In embodiments, the isolated CAR T-cell population is expanded for about 3 days to about 17 days. In embodiments, the isolated CAR T-cell population is expanded for about 3 days to about 16 days. In embodiments, the isolated CAR T-cell population is expanded for about 3 days to about 15 days. In embodiments, the isolated CAR T-cell population is expanded for about 3 days to about 14 days. In embodiments, the isolated CAR T-cell population is expanded for about 3 days to about 13 days. In embodiments, the isolated CAR T-cell population is expanded for about 3 days to about 12 days. In embodiments, the isolated CAR T-cell population is expanded for about 3 days to about 11 days. In embodiments, the isolated CAR T-cell population is expanded for about 3 days to about 10 days. In embodiments, the isolated CAR T-cell population is expanded for about 3 days to about 9 days. In embodiments, the isolated CAR T-cell population is expanded for about 3 days to about 8 days. In embodiments, the isolated CAR T-cell population is expanded for about 3 days to about 7 days. In embodiments, the isolated CAR T-cell population is expanded for about 3 days to about 6 days. In embodiments, the isolated CAR T-cell population is expanded for about 3 days to about 5 days. In embodiments, the isolated CAR T-cell population is expanded for about 3 days to about 4 days.

In embodiments, the isolated CAR T-cell population is expanded for 3 days to 60 days. In embodiments, the isolated CAR T-cell population is expanded for 4 days to 60 days. In embodiments, the isolated CAR T-cell population is expanded for 5 days to 60 days. In embodiments, the isolated CAR T-cell population is expanded for 6 days to 60 days. In embodiments, the isolated CAR T-cell population is expanded for 7 days to 60 days. In embodiments, the isolated CAR T-cell population is expanded for 8 days to 60 days. In embodiments, the isolated CAR T-cell population is expanded for 9 days to 60 days. In embodiments, the isolated CAR T-cell population is expanded for 10 days to 60 days. In embodiments, the isolated CAR T-cell population is expanded for 11 days to 60 days. In embodiments, the isolated CAR T-cell population is expanded for 12 days to 60 days. In embodiments, the isolated CAR T-cell population is expanded for 13 days to 60 days. In embodiments, the isolated CAR T-cell population is expanded for 14 days to 60 days. In embodiments, the isolated CAR T-cell population is expanded for 15 days to 60 days. In embodiments, the isolated CAR T-cell population is expanded for 16 days to 60 days. In embodiments, the isolated CAR T-cell population is expanded for 17 days to 60 days. In embodiments, the isolated CAR T-cell population is expanded for 18 days to 60 days. In embodiments, the isolated CAR T-cell population is expanded for 19 days to 60 days. In embodiments, the isolated CAR T-cell population is expanded for 20 days to 60 days. In embodiments, the isolated CAR T-cell population is expanded for 25 days to 60 days. In embodiments, the isolated CAR T-cell population is expanded for 30 days to 60 days. In embodiments, the isolated CAR T-cell population is expanded for 35 days to 60 days. In embodiments, the isolated CAR T-cell population is expanded for 40 days to 60 days. In embodiments, the isolated CAR T-cell population is expanded for 45 days to 60 days. In embodiments, the isolated CAR T-cell population is expanded for 50 days to 60 days. In embodiments, the isolated CAR T-cell population is expanded for 55 days to 60 days.

In embodiments, the isolated CAR T-cell population is expanded for 3 days to 55 days. In embodiments, the isolated CAR T-cell population is expanded for 3 days to 50 days. In embodiments, the isolated CAR T-cell population is expanded for 3 days to 45 days. In embodiments, the isolated CAR T-cell population is expanded for 3 days to 40 days. In embodiments, the isolated CAR T-cell population is expanded for 3 days to 35 days. In embodiments, the isolated CAR T-cell population is expanded for 3 days to 30 days. In embodiments, the isolated CAR T-cell population is expanded for 3 days to 25 days. In embodiments, the isolated CAR T-cell population is expanded for 3 days to 20 days. In embodiments, the isolated CAR T-cell population is expanded for 3 days to 19 days. In embodiments, the isolated CAR T-cell population is expanded for 3 days to 18 days. In embodiments, the isolated CAR T-cell population is expanded for 3 days to 17 days. In embodiments, the isolated CAR T-cell population is expanded for 3 days to 16 days. In embodiments, the isolated CAR T-cell population is expanded for 3 days to 15 days. In embodiments, the isolated CAR T-cell population is expanded for 3 days to 14 days. In embodiments, the isolated CAR T-cell population is expanded for 3 days to 13 days. In embodiments, the isolated CAR T-cell population is expanded for 3 days to 12 days. In embodiments, the isolated CAR T-cell population is expanded for 3 days to 11 days. In embodiments, the isolated CAR T-cell population is expanded for 3 days to 10 days. In embodiments, the isolated CAR T-cell population is expanded for 3 days to 9 days. In embodiments, the isolated CAR T-cell population is expanded for 3 days to 8 days. In embodiments, the isolated CAR T-cell population is expanded for 3 days to 7 days. In embodiments, the isolated CAR T-cell population is expanded for 3 days to 6 days. In embodiments, the isolated CAR T-cell population is expanded for 3 days to 5 days. In embodiments, the isolated CAR T-cell population is expanded for 3 days to 4 days.

In embodiments, the method further includes after the isolating a step (v) of administering a therapeutically effective amount of the purified CAR T-cell population to a subject in need thereof. In embodiments, the subject is a human. In embodiments, the subject is a cancer subject.

In embodiments, the cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer. In embodiments, the cancer is lymphoma. In embodiments, the cancer is chronic lymphocytic leukemia (CLL). In embodiments, the cancer is small lymphocytic lymphoma. In embodiments, the cancer is marginal cell B-Cell lymphoma. In embodiments, the cancer is Burkett's Lymphoma. In embodiments, the cancer is leukemia. In embodiments, the cancer is chronic B cell leukemia. In embodiments, the cancer is renal cell carcinoma. In embodiments, the cancer is colon cancer. In embodiments, the cancer is colorectal cancer. In embodiments, the cancer is breast cancer. In embodiments, the cancer is epithelial squamous cell cancer. In embodiments, the cancer is melanoma. In embodiments, the cancer is myeloma. In embodiments, the cancer is stomach cancer. In embodiments, the cancer is brain cancer. In embodiments, the cancer is lung cancer, pancreatic cancer. In embodiments, the cancer is cervical cancer. In embodiments, the cancer is ovarian cancer. In embodiments, the cancer is liver cancer. In embodiments, the cancer is bladder cancer. In embodiments, the cancer is prostate cancer. In embodiments, the cancer is testicular cancer. In embodiments, the cancer is thyroid cancer. In embodiments, the cancer is head and neck cancer.

In embodiments, the chimeric binding peptide is a labeled CAR-binding peptide.

In embodiments, the separating includes contacting the ligand-bound chimeric peptide-CAR T-cell complex with a magnetic particle or a solid support. In embodiments, the separating includes contacting the ligand-bound chimeric peptide-CAR T-cell complex with a magnetic particle. In embodiments, the separating includes contacting the ligand-bound chimeric peptide-CAR T-cell complex with a solid support.

In embodiments, the chimeric peptide-CAR T-cell complex is formed by binding of the CAR-binding domain to the CAR of the CAR-expressing T-cell. In embodiments, the binding of the CAR-binding domain does not activate a CAR T-cell at a detectable level. In embodiments, the ligand-bound chimeric peptide-CAR T-cell complex is formed by binding of the detectable moiety to the ligand. In embodiments, the detectable moiety is covalently or non-covalently bound to the chimeric binding peptide. In embodiments, the detectable moiety is covalently bound to the chimeric binding peptide. In embodiments, the detectable moiety is non-covalently bound to the chimeric binding peptide. In embodiments, the detectable moiety is a fluorescent moiety. In embodiments, the detectable moiety is a phycoerythrin (PE) moiety, a fluorescein isothiocyanate (FITC) moiety, or an Alexa Fluor 647 moiety. In embodiments, the detectable moiety is a phycoerythrin (PE) moiety. In embodiments, the detectable moiety is a fluorescein isothiocynate (FITC) moiety. In embodiments, the detectable moiety is an Alexa Fluor 647 moiety.

In embodiments, a plurality (e.g., at least 2) of detectable moieties are covalently or non-covalently bound to the chimeric binding peptide. Thus, in embodiments, the chimeric binding peptide includes a plurality of bound detectable moieties. In embodiments, a plurality of detectable moieties are covalently bound to the chimeric binding peptide. In embodiments, a plurality of detectable moieties are non-covalently bound to the chimeric binding peptide. In embodiments, the plurality of detectable moieties is a plurality of fluorescent moieties. In embodiments, the plurality of detectable moieties is a plurality of phycoerythrin (PE) moieties, a plurality fluorescein isothicynate (FITC) moieties, or a plurality of Alexa Fluor 647 moieties. In embodiments, the plurality of detectable moieties is a plurality of phycoerythrin (PE) moieities. In embodiments, the plurality of detectable moieties is a plurality of fluorescein isothicynate (FITC) moieties. In embodiments, the plurality of detectable moieties is a plurality of Alexa Fluor 647 moieties.

In embodiments, the detectable moiety is bound to an amino acid (e.g., an amino acid side chain) in the chimeric binding protein. In embodiments, the detectable moiety is bound to one or more amino acids (e.g., an amino acid side chain) in the chimeric binding protein. In embodiments, the detectable moiety is bound to one or more amino acids (e.g., one or more amino acid side chains) in the ligand binding domain, the CAR-binding domain and/or the chemical linker.

In embodiments, the detectable moiety is a FITC moiety. In further embodiments, the FITC moiety is bound to a primary amine in a lysine amino acid side chain in the chimeric binding peptide. In embodiments, the FITC moiety is bound to one or more primary amines in one or more lysine amino acid side chains in the chimeric binding peptide. In embodiments, the FITC moiety is bound to one or more primary amines in one or more lysine amino acid side chains in the ligand domain, the CAR-binding domain and/or the chemical linker.

In embodiments, the detectable moiety is an Alexa Fluor 647 moiety. In further embodiments, the Alexa Fluor 647 moiety is bound to a primary amine or a thiol group of an amino acid side chain in the chimeric binding protein. In embodiments, the Alexa Fluor 647 moiety is bound to one or more primary amines or thiol groups of one or more amino acid side chains in the chimeric binding peptide. In embodiments, the Alexa Fluor 647 moiety is bound to one or more primary amines or thiol groups of one or more amino acid side chains in the ligand binding domain, the CAR-binding domain and/or the chemical linker.

In embodiments, the ligand-bound chimeric peptide-CAR T-cell complex is formed by binding of the ligand binding domain to the ligand. In embodiments, the binding is covalent or non-covalent. In embodiments, the binding is covalent. In embodiments, the binding is non-covalent. In embodiments, the chimeric peptide-CAR T-cell complex includes one or more detectable moieties attached to the ligand binding domain, the CAR-binding domain and/or the chemical linker. Thus, the chimeric peptide-CAR T-cell complex may be referred to herein as a labeled CAR T-cell-peptide complex. In embodiments, the chimeric peptide-CAR T-cell complex is a labeled CAR T-cell-peptide complex.

In embodiments, the ligand is a protein, a nucleic acid, or a small molecule. In embodiments, the ligand is a protein. In embodiments, the ligand is a nucleic acid. In embodiments, the ligand is a small molecule. In embodiments, the ligand is an antibody domain.

In embodiments, the ligand is bound to a metal-coated particle or a solid support. In embodiments, the ligand is bound to a metal-coated particle. In embodiments, the ligand is bound to a solid support. In embodiments, the metal-coated particle is magnetic. In embodiments, the ligand binds a detectable moiety. In one further embodiment, the detectable moiety is a label. Thus, the ligand bound to a magnetic metal-coated particle may be referred to as a label-binding magnetic particle. In embodiments, the ligand is bound to a metal-coated particle thereby forming a label-binding magnetic particle.

In embodiments, the CAR-expressing T-cell is a ROR1 CAR T-cell, a ROR2 CAR T-cell, or a CD19 CAR T-cell. In embodiments, the CAR-expressing T-cell is a ROR1 CAR T-cell. In embodiments, the CAR-expressing T-cell is a ROR2 CAR T-cell. In embodiments, the CAR-expressing T-cell is a CD19 CAR T-cell.

In embodiments, the CAR-expressing T-cell is a TNFRSF17 CAR T-cell, a TNFRSF8 CAR T-cell, a CD5 CAR T-cell, a ULBP1 CAR T-cell, a ULBP2 CAR T-cell, a CEACAM5 CAR T-cell, a CD276 CAR T-cell, an IL3RA CAR T-cell, a MS4A1 CAR T-cell, a CD33 CAR T-cell, a NCAM1 CAR T-cell, a MET CAR T-cell, a CSPG4 CAR T-cell, a SDC1 CAR T-cell, a CD22 CAR T-cell, a CD38 CAR T-cell, a CD70 CAR T-cell, an IL1RAP CAR T-cell, an EGFR CAR T-cell, a CD133 CAR T-cell, an EGFRvIII CAR T-cell, an EPCAM CAR T-cell, an EPHA2 CAR T-cell, an ERBB2 CAR T-cell, a GPC3 CAR T-cell, a MSLN CAR T-cell, a TEM1 CAR T-cell, a MUC1 CAR T-cell, a PDCD1 CAR T-cell, a CD274 CAR T-cell, a KDR CAR T-cell, an IL13RA2 CAR T-cell, a FOLH1 CAR T-cell, a GPNMB CAR T-cell, a FAP CAR T-cell, a CA9 CAR T-cell, a FOLR1 CAR T-cell, a L1CAM CAR T-cell, a CD23 CAR T-cell, a PSCA CAR T-cell, a CD44 CAR T-cell, a CD174 CAR T-cell, a SLAMF7 CAR T-cell, a GD2 CAR T-cell, a BCMA CAR T-cell, a CD30 CAR T-cell, a CD4 CAR T-cell, a CD7 CAR T-cell, a CD79B CAR T-cell, a CLEC12A CAR T-cell, a Siglec-2 CAR T-cell, a Siglec-3 CAR T-cell, a SIRP alpha CAR T-cell, a uPAR CAR T-cell, a VEGFR2 CAR T-cell, a GUCY2C CAR T-cell, or a Protein L CAR T-cell.

In embodiments, the CAR-expressing T-cell is a TNFRSF17 CAR T-cell. In embodiments, the CAR-expressing T-cell is a TNFRSF8 CAR T-cell. In embodiments, the CAR-expressing T-cell is a CD5 CAR T-cell. In embodiments, the CAR-expressing T-cell is a ULBP1 CAR T-cell. In embodiments, the CAR-expressing T-cell is a ULBP2 CAR T-cell. In embodiments, the CAR-expressing T-cell is a CEACAM5 CAR T-cell. In embodiments, the CAR-expressing T-cell is a CD276 CAR T-cell. In embodiments, the CAR-expressing T-cell is a IL3RA CAR T-cell. In embodiments, the CAR-expressing T-cell is a MS4A1 CAR T-cell. In embodiments, the CAR-expressing T-cell is a CD33 CAR T-cell. In embodiments, the CAR-expressing T-cell is a NCAM1 CAR T-cell. In embodiments, the CAR-expressing T-cell is a MET CAR T-cell. In embodiments, the CAR-expressing T-cell is a CSPG4 CAR T-cell. In embodiments, the CAR-expressing T-cell is a SDC1 CAR T-cell. In embodiments, the CAR-expressing T-cell is a CD22 CAR T-cell. In embodiments, the CAR-expressing T-cell is a CD38 CAR T-cell. In embodiments, the CAR-expressing T-cell is a CD70 CAR T-cell. In embodiments, the CAR-expressing T-cell is an IL1RAP CAR T-cell. In embodiments, the CAR-expressing T-cell is an EGFR CAR T-cell. In embodiments, the CAR-expressing T-cell is a CD133 CAR T-cell. In embodiments, the CAR-expressing T-cell is an EGFRVIII CAR T-cell. In embodiments, the CAR-expressing T-cell is an EPCAM CAR T-cell. In embodiments, the CAR-expressing T-cell is an EPHA2 CAR T-cell. In embodiments, the CAR-expressing T-cell is an ERBB2 CAR T-cell. In embodiments, the CAR-expressing T-cell is a GPC3 CAR T-cell. In embodiments, the CAR-expressing T-cell is a MSLN CAR T-cell. In embodiments, the CAR-expressing T-cell is a TEM1 CAR T-cell. In embodiments, the CAR-expressing T-cell is a MUC1 CAR T-cell. In embodiments, the CAR-expressing T-cell is a PDCD1 CAR T-cell. In embodiments, the CAR-expressing T-cell is a CD274 CAR T-cell. In embodiments, the CAR-expressing T-cell is a KDR CAR T-cell. In embodiments, the CAR-expressing T-cell is an IL13RA2 CAR T-cell. In embodiments, the CAR-expressing T-cell is a FOLH1 CAR T-cell. In embodiments, the CAR-expressing T-cell is a GPNMB CAR T-cell. In embodiments, the CAR-expressing T-cell is a FAP CAR T-cell. In embodiments, the CAR-expressing T-cell is a CA9 CAR T-cell. In embodiments, the CAR-expressing T-cell is a FOLR1 CAR T-cell. In embodiments, the CAR-expressing T-cell is a L1CAM CAR T-cell, a CD23 CAR T-cell. In embodiments, the CAR-expressing T-cell is a PSCA CAR T-cell. In embodiments, the CAR-expressing T-cell is a CD44 CAR T-cell. In embodiments, the CAR-expressing T-cell is a CD174 CAR T-cell. In embodiments, the CAR-expressing T-cell is a SLAMF7 CAR T-cell. In embodiments, the CAR-expressing T-cell is a GD2 CAR T-cell. In embodiments, the CAR-expressing T-cell is a BCMA CAR T-cell. In embodiments, the CAR-expressing T-cell is a CD30 CAR T-cell. In embodiments, the CAR-expressing T-cell is a CD4 CAR T-cell. In embodiments, the CAR-expressing T-cell is a CD7 CAR T-cell. In embodiments, the CAR-expressing T-cell is a CD79B CAR T-cell. In embodiments, the CAR-expressing T-cell is a CLEC12A CAR T-cell. In embodiments, the CAR-expressing T-cell is a Siglec-2 CAR T-cell. In embodiments, the CAR-expressing T-cell is a Siglec-3 CAR T-cell. In embodiments, the CAR-expressing T-cell is a SIRP alpha CAR T-cell. In embodiments, the CAR-expressing T-cell is a uPAR CAR T-cell, a VEGFR2 CAR T-cell. In embodiments, the CAR-expressing T-cell is a GUCY2C CAR T-cell. In embodiments, the CAR-expressing T-cell is a Protein L CAR T-cell.

In embodiments, the CAR-binding domain and the ligand binding domain are connected through a chemical linker. In embodiments, the chemical linker is a peptide.

The chimeric binding peptide provided herein is a polypeptide including a first domain specifically binding to a CAR and a second domain specifically binding to a ligand used to physically separate the CAR expressing T-cell from the remainder of the cell population. The CAR binding domain may therefore be an epitope specifically bound by the CAR. The ligand binding domain on the other hand may be a peptide domain that specifically binds to an antibody used for separation purposes. The first and second domain may be directly attached to each other or they may be bound to each other by a chemical linker. A “chemical linker,” as provided herein, is a covalent linker, a non-covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered,) heteroarylene.

In embodiments, the chemical linker is a covalent linker. In embodiments, the chemical linker is a hydrocarbon linker. In embodiments, the chemical linker is a cleavable peptide linker.

Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

In embodiments, the chemical linker includes an amino acid sequence of about 2 about 25 amino acids in length. In embodiments, the chemical linker includes an amino acid sequence of about 3 about 25 amino acids in length. In embodiments, the chemical linker includes an amino acid sequence of about 4 about 25 amino acids in length. In embodiments, the chemical linker includes an amino acid sequence of about 5 about 25 amino acids in length. In embodiments, the chemical linker includes an amino acid sequence of about 6 about 25 amino acids in length. In embodiments, the chemical linker includes an amino acid sequence of about 7 about 25 amino acids in length. In embodiments, the chemical linker includes an amino acid sequence of about 8 about 25 amino acids in length. In embodiments, the chemical linker includes an amino acid sequence of about 9 about 25 amino acids in length. In embodiments, the chemical linker includes an amino acid sequence of about 10 about 25 amino acids in length. In embodiments, the chemical linker includes an amino acid sequence of about 11 about 25 amino acids in length. In embodiments, the chemical linker includes an amino acid sequence of about 12 about 25 amino acids in length. In embodiments, the chemical linker includes an amino acid sequence of about 13 about 25 amino acids in length. In embodiments, the chemical linker includes an amino acid sequence of about 14 about 25 amino acids in length. In embodiments, the chemical linker includes an amino acid sequence of about 15 about 25 amino acids in length. In embodiments, the chemical linker includes an amino acid sequence of about 16 about 25 amino acids in length. In embodiments, the chemical linker includes an amino acid sequence of about 17 about 25 amino acids in length. In embodiments, the chemical linker includes an amino acid sequence of about 18 about 25 amino acids in length. In embodiments, the chemical linker includes an amino acid sequence of about 19 about 25 amino acids in length. In embodiments, the chemical linker includes an amino acid sequence of about 20 about 25 amino acids in length. In embodiments, the chemical linker includes an amino acid sequence of about 21 about 25 amino acids in length. In embodiments, the chemical linker includes an amino acid sequence of about 22 about 25 amino acids in length. In embodiments, the chemical linker includes an amino acid sequence of about 23 about 25 amino acids in length. In embodiments, the chemical linker includes an amino acid sequence of about 24 about 25 amino acids in length.

In embodiments, the chemical linker includes an amino acid sequence of about 2 about 24 amino acids in length. In embodiments, the chemical linker includes an amino acid sequence of about 2 about 23 amino acids in length. In embodiments, the chemical linker includes an amino acid sequence of about 2 about 22 amino acids in length. In embodiments, the chemical linker includes an amino acid sequence of about 2 about 21 amino acids in length. In embodiments, the chemical linker includes an amino acid sequence of about 2 about 20 amino acids in length. In embodiments, the chemical linker includes an amino acid sequence of about 2 about 19 amino acids in length. In embodiments, the chemical linker includes an amino acid sequence of about 2 about 18 amino acids in length. In embodiments, the chemical linker includes an amino acid sequence of about 2 about 17 amino acids in length. In embodiments, the chemical linker includes an amino acid sequence of about 2 about 16 amino acids in length. In embodiments, the chemical linker includes an amino acid sequence of about 2 about 15 amino acids in length. In embodiments, the chemical linker includes an amino acid sequence of about 2 about 14 amino acids in length. In embodiments, the chemical linker includes an amino acid sequence of about 2 about 13 amino acids in length. In embodiments, the chemical linker includes an amino acid sequence of about 2 about 12 amino acids in length. In embodiments, the chemical linker includes an amino acid sequence of about 2 about 11 amino acids in length. In embodiments, the chemical linker includes an amino acid sequence of about 2 about 10 amino acids in length. In embodiments, the chemical linker includes an amino acid sequence of about 2 about 9 amino acids in length. In embodiments, the chemical linker includes an amino acid sequence of about 2 about 8 amino acids in length. In embodiments, the chemical linker includes an amino acid sequence of about 2 about 7 amino acids in length. In embodiments, the chemical linker includes an amino acid sequence of about 2 about 6 amino acids in length. In embodiments, the chemical linker includes an amino acid sequence of about 2 about 5 amino acids in length. In embodiments, the chemical linker includes an amino acid sequence of about 2 about 4 amino acids in length. In embodiments, the chemical linker includes an amino acid sequence of about 2 about 3 amino acids in length.

In embodiments, the chemical linker is an amino acid sequence of 2 to 25 amino acids in length. In embodiments, the chemical linker is an amino acid sequence of 3 to 25 amino acids in length. In embodiments, the chemical linker is an amino acid sequence of 4 to 25 amino acids in length. In embodiments, the chemical linker is an amino acid sequence of 5 to 25 amino acids in length. In embodiments, the chemical linker is an amino acid sequence of 6 to 25 amino acids in length. In embodiments, the chemical linker is an amino acid sequence of 7 to 25 amino acids in length. In embodiments, the chemical linker is an amino acid sequence of 8 to 25 amino acids in length. In embodiments, the chemical linker is an amino acid sequence of 9 to 25 amino acids in length. In embodiments, the chemical linker is an amino acid sequence of 10 to 25 amino acids in length. In embodiments, the chemical linker is an amino acid sequence of 11 to 25 amino acids in length. In embodiments, the chemical linker is an amino acid sequence of 12 to 25 amino acids in length. In embodiments, the chemical linker is an amino acid sequence of 13 to 25 amino acids in length. In embodiments, the chemical linker is an amino acid sequence of 14 to 25 amino acids in length. In embodiments, the chemical linker is an amino acid sequence of 15 to 25 amino acids in length. In embodiments, the chemical linker is an amino acid sequence of 16 to 25 amino acids in length. In embodiments, the chemical linker is an amino acid sequence of 17 to 25 amino acids in length. In embodiments, the chemical linker is an amino acid sequence of 18 to 25 amino acids in length. In embodiments, the chemical linker is an amino acid sequence of 19 to 25 amino acids in length. In embodiments, the chemical linker is an amino acid sequence of 20 to 25 amino acids in length. In embodiments, the chemical linker is an amino acid sequence of 21 to 25 amino acids in length. In embodiments, the chemical linker is an amino acid sequence of 22 to 25 amino acids in length. In embodiments, the chemical linker is an amino acid sequence of 23 to 25 amino acids in length. In embodiments, the chemical linker is an amino acid sequence of 24 to 25 amino acids in length.

In embodiments, the chemical linker includes an amino acid sequence of 2 to 24 amino acids in length. In embodiments, the chemical linker includes an amino acid sequence of 2 to 23 amino acids in length. In embodiments, the chemical linker includes an amino acid sequence of 2 to 22 amino acids in length. In embodiments, the chemical linker includes an amino acid sequence of 2 to 21 amino acids in length. In embodiments, the chemical linker includes an amino acid sequence of 2 to 20 amino acids in length. In embodiments, the chemical linker includes an amino acid sequence of 2 to 19 amino acids in length. In embodiments, the chemical linker includes an amino acid sequence of 2 to 18 amino acids in length. In embodiments, the chemical linker includes an amino acid sequence of 2 to 17 amino acids in length. In embodiments, the chemical linker includes an amino acid sequence of 2 to 16 amino acids in length. In embodiments, the chemical linker includes an amino acid sequence of 2 to 15 amino acids in length. In embodiments, the chemical linker includes an amino acid sequence of 2 to 14 amino acids in length. In embodiments, the chemical linker includes an amino acid sequence of 2 to 13 amino acids in length. In embodiments, the chemical linker includes an amino acid sequence of 2 to 12 amino acids in length. In embodiments, the chemical linker includes an amino acid sequence of 2 to 11 amino acids in length. In embodiments, the chemical linker includes an amino acid sequence of 2 to 10 amino acids in length. In embodiments, the chemical linker includes an amino acid sequence of 2 to 9 amino acids in length. In embodiments, the chemical linker includes an amino acid sequence of 2 to 8 amino acids in length. In embodiments, the chemical linker includes an amino acid sequence of 2 to 7 amino acids in length. In embodiments, the chemical linker includes an amino acid sequence of 2 to 6 amino acids in length. In embodiments, the chemical linker includes an amino acid sequence of 2 to 5 amino acids in length. In embodiments, the chemical linker includes an amino acid sequence of 2 to 4 amino acids in length. In embodiments, the chemical linker includes an amino acid sequence of 2 to 3 amino acids in length.

In embodiments, the chemical linker includes the amino acid sequence of SEQ ID NO: 2. In embodiments, the chemical linker is the amino acid sequence of SEQ ID NO:2.

In embodiments, the chimeric binding peptide includes an amino acid sequence of about 25 to about 1000 amino acids in length. In embodiments, the chimeric binding peptide includes an amino acid sequence of about 50 to about 1000 amino acids in length. In embodiments, the chimeric binding peptide includes an amino acid sequence of about 75 to about 1000 amino acids in length. In embodiments, the chimeric binding peptide includes an amino acid sequence of about 100 to about 1000 amino acids in length. In embodiments, the chimeric binding peptide includes an amino acid sequence of about 150 to about 1000 amino acids in length. In embodiments, the chimeric binding peptide includes an amino acid sequence of about 200 to about 1000 amino acids in length. In embodiments, the chimeric binding peptide includes an amino acid sequence of about 250 to about 1000 amino acids in length. In embodiments, the chimeric binding peptide includes an amino acid sequence of about 300 to about 1000 amino acids in length. In embodiments, the chimeric binding peptide includes an amino acid sequence of about 350 to about 1000 amino acids in length. In embodiments, the chimeric binding peptide includes an amino acid sequence of about 400 to about 1000 amino acids in length. In embodiments, the chimeric binding peptide includes an amino acid sequence of about 450 to about 1000 amino acids in length. In embodiments, the chimeric binding peptide includes an amino acid sequence of about 500 to about 1000 amino acids in length. In embodiments, the chimeric binding peptide includes an amino acid sequence of about 550 to about 1000 amino acids in length. In embodiments, the chimeric binding peptide includes an amino acid sequence of about 600 to about 1000 amino acids in length. In embodiments, the chimeric binding peptide includes an amino acid sequence of about 650 to about 1000 amino acids in length. In embodiments, the chimeric binding peptide includes an amino acid sequence of about 700 to about 1000 amino acids in length. In embodiments, the chimeric binding peptide includes an amino acid sequence of about 750 to about 1000 amino acids in length. In embodiments, the chimeric binding peptide includes an amino acid sequence of about 800 to about 1000 amino acids in length. In embodiments, the chimeric binding peptide includes an amino acid sequence of about 850 to about 1000 amino acids in length. In embodiments, the chimeric binding peptide includes an amino acid sequence of about 900 to about 1000 amino acids in length. In embodiments, the chimeric binding peptide includes an amino acid sequence of about 950 to about 1000 amino acids in length.

In embodiments, the chimeric binding peptide includes an amino acid sequence of about 25 to about 950 amino acids in length. In embodiments, the chimeric binding peptide includes an amino acid sequence of about 25 to about 900 amino acids in length. In embodiments, the chimeric binding peptide includes an amino acid sequence of about 25 to about 850 amino acids in length. In embodiments, the chimeric binding peptide includes an amino acid sequence of about 25 to about 800 amino acids in length. In embodiments, the chimeric binding peptide includes an amino acid sequence of about 25 to about 750 amino acids in length. In embodiments, the chimeric binding peptide includes an amino acid sequence of about 25 to about 700 amino acids in length. In embodiments, the chimeric binding peptide includes an amino acid sequence of about 25 to about 650 amino acids in length. In embodiments, the chimeric binding peptide includes an amino acid sequence of about 25 to about 600 amino acids in length. In embodiments, the chimeric binding peptide includes an amino acid sequence of about 25 to about 550 amino acids in length. In embodiments, the chimeric binding peptide includes an amino acid sequence of about 25 to about 500 amino acids in length. In embodiments, the chimeric binding peptide includes an amino acid sequence of about 25 to about 450 amino acids in length. In embodiments, the chimeric binding peptide includes an amino acid sequence of about 25 to about 400 amino acids in length. In embodiments, the chimeric binding peptide includes an amino acid sequence of about 25 to about 350 amino acids in length. In embodiments, the chimeric binding peptide includes an amino acid sequence of about 25 to about 300 amino acids in length. In embodiments, the chimeric binding peptide includes an amino acid sequence of about 25 to about 250 amino acids in length. In embodiments, the chimeric binding peptide includes an amino acid sequence of about 25 to about 200 amino acids in length. In embodiments, the chimeric binding peptide includes an amino acid sequence of about 25 to about 150 amino acids in length. In embodiments, the chimeric binding peptide includes an amino acid sequence of about 25 to about 100 amino acids in length. In embodiments, the chimeric binding peptide includes an amino acid sequence of about 25 to about 75 amino acids in length. In embodiments, the chimeric binding peptide includes an amino acid sequence of about 25 to about 50 amino acids in length.

In embodiments, the chimeric binding peptide includes an amino acid sequence of 25 to 1000 amino acids in length. In embodiments, the chimeric binding peptide includes an amino acid sequence of 50 to 1000 amino acids in length. In embodiments, the chimeric binding peptide includes an amino acid sequence of 75 to 1000 amino acids in length. In embodiments, the chimeric binding peptide includes an amino acid sequence of 100 to 1000 amino acids in length. In embodiments, the chimeric binding peptide includes an amino acid sequence of 150 to 1000 amino acids in length. In embodiments, the chimeric binding peptide includes an amino acid sequence of 200 to 1000 amino acids in length. In embodiments, the chimeric binding peptide includes an amino acid sequence of 250 to 1000 amino acids in length. In embodiments, the chimeric binding peptide includes an amino acid sequence of 300 to 1000 amino acids in length. In embodiments, the chimeric binding peptide includes an amino acid sequence of 350 to 1000 amino acids in length. In embodiments, the chimeric binding peptide includes an amino acid sequence of 400 to 1000 amino acids in length. In embodiments, the chimeric binding peptide includes an amino acid sequence of 450 to 1000 amino acids in length. In embodiments, the chimeric binding peptide includes an amino acid sequence of 500 to 1000 amino acids in length. In embodiments, the chimeric binding peptide includes an amino acid sequence of 550 to 1000 amino acids in length. In embodiments, the chimeric binding peptide includes an amino acid sequence of 600 to 1000 amino acids in length. In embodiments, the chimeric binding peptide includes an amino acid sequence of 650 to 1000 amino acids in length. In embodiments, the chimeric binding peptide includes an amino acid sequence of 700 to 1000 amino acids in length. In embodiments, the chimeric binding peptide includes an amino acid sequence of 750 to 1000 amino acids in length. In embodiments, the chimeric binding peptide includes an amino acid sequence of 800 to 1000 amino acids in length. In embodiments, the chimeric binding peptide includes an amino acid sequence of 850 to 1000 amino acids in length. In embodiments, the chimeric binding peptide includes an amino acid sequence of 900 to 1000 amino acids in length. In embodiments, the chimeric binding peptide includes an amino acid sequence of 950 to 1000 amino acids in length.

In embodiments, the chimeric binding peptide includes an amino acid sequence of 25 to 950 amino acids in length. In embodiments, the chimeric binding peptide includes an amino acid sequence of 25 to 900 amino acids in length. In embodiments, the chimeric binding peptide includes an amino acid sequence of 25 to 850 amino acids in length. In embodiments, the chimeric binding peptide includes an amino acid sequence of 25 to 800 amino acids in length. In embodiments, the chimeric binding peptide includes an amino acid sequence of 25 to 750 amino acids in length. In embodiments, the chimeric binding peptide includes an amino acid sequence of 25 to 700 amino acids in length. In embodiments, the chimeric binding peptide includes an amino acid sequence of 25 to 650 amino acids in length. In embodiments, the chimeric binding peptide includes an amino acid sequence of 25 to 600 amino acids in length. In embodiments, the chimeric binding peptide includes an amino acid sequence of 25 to 550 amino acids in length. In embodiments, the chimeric binding peptide includes an amino acid sequence of 25 to 500 amino acids in length. In embodiments, the chimeric binding peptide includes an amino acid sequence of 25 to 450 amino acids in length. In embodiments, the chimeric binding peptide includes an amino acid sequence of 25 to 400 amino acids in length. In embodiments, the chimeric binding peptide includes an amino acid sequence of 25 to 350 amino acids in length. In embodiments, the chimeric binding peptide includes an amino acid sequence of 25 to 300 amino acids in length. In embodiments, the chimeric binding peptide includes an amino acid sequence of 25 to 250 amino acids in length. In embodiments, the chimeric binding peptide includes an amino acid sequence of 25 to 200 amino acids in length. In embodiments, the chimeric binding peptide includes an amino acid sequence of 25 to 150 amino acids in length. In embodiments, the chimeric binding peptide includes an amino acid sequence of 25 to 100 amino acids in length. In embodiments, the chimeric binding peptide includes an amino acid sequence of 25 to 75 amino acids in length. In embodiments, the chimeric binding peptide includes an amino acid sequence of 25 to 50 amino acids in length.

In embodiments, CAR-binding domain includes an amino acid sequence of about 25 to about 500 amino acids in length. In embodiments, CAR-binding domain includes an amino acid sequence of about 50 to about 500 amino acids in length. In embodiments, CAR-binding domain includes an amino acid sequence of about 75 to about 500 amino acids in length. In embodiments, CAR-binding domain includes an amino acid sequence of about 100 to about 500 amino acids in length. In embodiments, CAR-binding domain includes an amino acid sequence of about 150 to about 500 amino acids in length. In embodiments, CAR-binding domain includes an amino acid sequence of about 200 to about 500 amino acids in length. In embodiments, CAR-binding domain includes an amino acid sequence of about 250 to about 500 amino acids in length. In embodiments, CAR-binding domain includes an amino acid sequence of about 300 to about 500 amino acids in length. In embodiments, CAR-binding domain includes an amino acid sequence of about 350 to about 500 amino acids in length. In embodiments, CAR-binding domain includes an amino acid sequence of about 400 to about 500 amino acids in length. In embodiments, CAR-binding domain includes an amino acid sequence of about 450 to about 500 amino acids in length.

In embodiments, CAR-binding domain includes an amino acid sequence of about 25 to about 450 amino acids in length. In embodiments, CAR-binding domain includes an amino acid sequence of about 25 to about 400 amino acids in length. In embodiments, CAR-binding domain includes an amino acid sequence of about 25 to about 350 amino acids in length. In embodiments, CAR-binding domain includes an amino acid sequence of about 25 to about 300 amino acids in length. In embodiments, CAR-binding domain includes an amino acid sequence of about 25 to about 250 amino acids in length. In embodiments, CAR-binding domain includes an amino acid sequence of about 25 to about 200 amino acids in length. In embodiments, CAR-binding domain includes an amino acid sequence of about 25 to about 150 amino acids in length. In embodiments, CAR-binding domain includes an amino acid sequence of about 25 to about 100 amino acids in length. In embodiments, CAR-binding domain includes an amino acid sequence of about 25 to about 75 amino acids in length. In embodiments, CAR-binding domain includes an amino acid sequence of about 25 to about 50 amino acids in length.

In embodiments, CAR-binding domain includes an amino acid sequence of 25 to 500 amino acids in length. In embodiments, CAR-binding domain includes an amino acid sequence of 50 to 500 amino acids in length. In embodiments, CAR-binding domain includes an amino acid sequence of 75 to 500 amino acids in length. In embodiments, CAR-binding domain includes an amino acid sequence of 100 to 500 amino acids in length. In embodiments, CAR-binding domain includes an amino acid sequence of 150 to 500 amino acids in length. In embodiments, CAR-binding domain includes an amino acid sequence of 200 to 500 amino acids in length. In embodiments, CAR-binding domain includes an amino acid sequence of 250 to 500 amino acids in length. In embodiments, CAR-binding domain includes an amino acid sequence of 300 to 500 amino acids in length. In embodiments, CAR-binding domain includes an amino acid sequence of 350 to 500 amino acids in length. In embodiments, CAR-binding domain includes an amino acid sequence of 400 to 500 amino acids in length. In embodiments, CAR-binding domain includes an amino acid sequence of 450 to 500 amino acids in length.

In embodiments, CAR-binding domain includes an amino acid sequence of 25 to 450 amino acids in length. In embodiments, CAR-binding domain includes an amino acid sequence of 25 to 400 amino acids in length. In embodiments, CAR-binding domain includes an amino acid sequence of 25 to 350 amino acids in length. In embodiments, CAR-binding domain includes an amino acid sequence of 25 to 300 amino acids in length. In embodiments, CAR-binding domain includes an amino acid sequence of 25 to 250 amino acids in length. In embodiments, CAR-binding domain includes an amino acid sequence of 25 to 200 amino acids in length. In embodiments, CAR-binding domain includes an amino acid sequence of 25 to 150 amino acids in length. In embodiments, CAR-binding domain includes an amino acid sequence of 25 to 100 amino acids in length. In embodiments, CAR-binding domain includes an amino acid sequence of 25 to 75 amino acids in length. In embodiments, CAR-binding domain includes an amino acid sequence of 25 to 50 amino acids in length.

In embodiments, the CAR-binding domain is a cancer antigen or fragment thereof. In embodiments, the CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme. In embodiments, the CAR-binding domain is a receptor tyrosine kinase domain. In embodiments, the CAR-binding domain is a tumor necrosis factor (TNF) receptor. In embodiments, the CAR-binding domain is an interleukin cytokine. In embodiments, the CAR-binding domain is an interleukin receptor. In embodiments, the CAR-binding domain is a non-classical serine protease. In embodiments, the CAR-binding domain is a B cell surface molecule. In embodiments, the CAR-binding domain is a T-cell surface molecule. In embodiments, the CAR-binding domain is a natural killer (NK) cell surface molecule. In embodiments, the CAR-binding domain is a mucin. In embodiments, the CAR-binding domain is a cell adhesion molecule. In embodiments, the CAR-binding domain is a disialoganglioside. In embodiments, the CAR-binding domain is a C-type lectin-like receptor. In embodiments, the CAR-binding domain is a tight junction protein. In embodiments, the CAR-binding domain is a Lewis antigen. In embodiments, the CAR-binding domain is a zinc metalloenzyme.

In embodiments, the CAR binding domain is a ROR domain. In embodiments, the CAR-binding domain is a ROR1 domain. In embodiments, the CAR-binding domain is a ROR1 Ig-like domain. In embodiments, the CAR-binding domain includes the amino acid sequence of SEQ ID NO:1. In embodiments, the CAR-binding domain is the amino acid sequence of SEQ ID NO: 1.

In embodiments, the CAR binding domain is a ROR2 domain. In embodiments, the CAR-binding domain is a ROR2 Ig-like domain. In embodiments, the CAR-binding domain is a CD19 domain.

In embodiments, the CAR-binding domain is a TNFRSF17 domain, a TNFRSF8 domain, a CD5 domain, a ULBP1 domain, a ULBP2 domain, a CEACAM5 domain, a CD276 domain, an IL3RA domain, a MS4A1 domain, a CD33 domain, a NCAM1 domain, a MET domain, a CSPG4 domain, a SDC1 domain, a CD22 domain, a CD38 domain, a CD70 domain, an IL1RAP domain, an EGFR domain, a CD133 domain, an EGFRvIII domain, an EPCAM domain, an EPHA2 domain, an ERBB2 domain, a GPC3 domain, a MSLN domain, a TEM1 domain, a MUC1 domain, a PDCD1 domain, a CD274 domain, a KDR domain, an IL13RA2 domain, a FOLH1 domain, a GPNMB domain, a FAP domain, a CA9 domain, a FOLR1 domain, a L1CAM domain, a CD23 domain, a PSCA domain, a CD44 domain, a CD174 domain, a SLAMF7 domain, a GD2 domain, a BCMA domain, a CD30 domain, a CD4 domain, a CD7 domain, a CD79B domain, a CLEC12A domain, a Siglec-2 domain, a Siglec-3 domain, a SIRP alpha domain, a uPAR domain, a VEGFR2 domain, a GUCY2C domain, or a Protein L domain.

In embodiments, the CAR-binding domain is a TNFRSF17 domain. In embodiments, the CAR-binding domain is a TNFRSF8 domain. In embodiments, the CAR-binding domain is a CD5 domain. In embodiments, the CAR-binding domain is a ULBP1 domain. In embodiments, the CAR-binding domain is a ULBP2 domain. In embodiments, the CAR-binding domain is a CEACAM5 domain. In embodiments, the CAR-binding domain is a CD276 domain. In embodiments, the CAR-binding domain is an IL3RA domain. In embodiments, the CAR-binding domain is a MS4A1 domain. In embodiments, the CAR-binding domain is a CD33 domain. In embodiments, the CAR-binding domain is a NCAM1 domain. In embodiments, the CAR-binding domain is a MET domain. In embodiments, the CAR-binding domain is a CSPG4 domain. In embodiments, the CAR-binding domain is a SDC1 domain. In embodiments, the CAR-binding domain is a CD22 domain. In embodiments, the CAR-binding domain is a CD38 domain. In embodiments, the CAR-binding domain is a CD70 domain. In embodiments, the CAR-binding domain is an IL1RAP Domain. In embodiments, the CAR-binding domain is an EGFR domain. In embodiments, the CAR-binding domain is a CD133 domain. In embodiments, the CAR-binding domain is an EGFRvIII domain. In embodiments, the CAR-binding domain is an EPCAM domain. In embodiments, the CAR-binding domain is an EPHA2 domain. In embodiments, the CAR-binding domain is an ERBB2 domain. In embodiments, the CAR-binding domain is a GPC3 domain. In embodiments, the CAR-binding domain is a MSLN domain. In embodiments, the CAR-binding domain is a TEM1 domain. In embodiments, the CAR-binding domain is a MUC1 domain. In embodiments, the CAR-binding domain is a PDCD1 domain. In embodiments, the CAR-binding domain is a CD274 domain. In embodiments, the CAR-binding domain is a KDR domain. In embodiments, the CAR-binding domain is an IL13RA2 domain. In embodiments, the CAR-binding domain is a FOLH1 domain. In embodiments, the CAR-binding domain is a GPNMB domain. In embodiments, the CAR-binding domain is a FAP domain. In embodiments, the CAR-binding domain is a CA9 domain. In embodiments, the CAR-binding domain is a FOLR1 domain. In embodiments, the CAR-binding domain is a L1CAM domain, a CD23 domain. In embodiments, the CAR-binding domain is a PSCA domain. In embodiments, the CAR-binding domain is a CD44 domain. In embodiments, the CAR-binding domain is a CD174 domain. In embodiments, the CAR-binding domain is a SLAMF7 domain. In embodiments, the CAR-binding domain is a GD2 domain. In embodiments, the CAR-binding domain is a BCMA domain. In embodiments, the CAR-binding domain is a CD30 domain. In embodiments, the CAR-binding domain is a CD4 domain. In embodiments, the CAR-binding domain is a CD7 domain. In embodiments, the CAR-binding domain is a CD79B domain. In embodiments, the CAR-binding domain is a CLEC12A domain. In embodiments, the CAR-binding domain is a Siglec-2 domain. In embodiments, the CAR-binding domain is a Siglec-3 domain. In embodiments, the CAR-binding domain is a SIRP alpha domain. In embodiments, the CAR-binding domain is a uPAR domain, a VEGFR2 domain. In embodiments, the CAR-binding domain is a GUCY2C domain. In embodiments, the CAR-binding domain is a Protein L domain.

In embodiments, the ligand binding domain includes an amino acid sequence of about 1 to about 500 amino acids in length. In embodiments, the ligand binding domain includes an amino acid sequence of about 2 to about 500 amino acids in length. In embodiments, the ligand binding domain includes an amino acid sequence of about 3 to about 500 amino acids in length. In embodiments, the ligand binding domain includes an amino acid sequence of about 4 to about 500 amino acids in length. In embodiments, the ligand binding domain includes an amino acid sequence of about 5 to about 500 amino acids in length. In embodiments, the ligand binding domain includes an amino acid sequence of about 6 to about 500 amino acids in length. In embodiments, the ligand binding domain includes an amino acid sequence of about 7 to about 500 amino acids in length. In embodiments, the ligand binding domain includes an amino acid sequence of about 8 to about 500 amino acids in length. In embodiments, the ligand binding domain includes an amino acid sequence of about 9 to about 500 amino acids in length. In embodiments, the ligand binding domain includes an amino acid sequence of about 10 to about 500 amino acids in length. In embodiments, the ligand binding domain includes an amino acid sequence of about 15 to about 500 amino acids in length. In embodiments, the ligand binding domain includes an amino acid sequence of about 20 to about 500 amino acids in length. In embodiments, the ligand binding domain includes an amino acid sequence of about 25 to about 500 amino acids in length. In embodiments, the ligand binding domain includes an amino acid sequence of about 50 to about 500 amino acids in length. In embodiments, the ligand binding domain includes an amino acid sequence of about 75 to about 500 amino acids in length. In embodiments, the ligand binding domain includes an amino acid sequence of about 100 to about 500 amino acids in length. In embodiments, the ligand binding domain includes an amino acid sequence of about 150 to about 500 amino acids in length. In embodiments, the ligand binding domain includes an amino acid sequence of about 200 to about 500 amino acids in length. In embodiments, the ligand binding domain includes an amino acid sequence of about 250 to about 500 amino acids in length. In embodiments, the ligand binding domain includes an amino acid sequence of about 300 to about 500 amino acids in length. In embodiments, the ligand binding domain includes an amino acid sequence of about 350 to about 500 amino acids in length. In embodiments, the ligand binding domain includes an amino acid sequence of about 400 to about 500 amino acids in length. In embodiments, the ligand binding domain includes an amino acid sequence of about 450 to about 500 amino acids in length.

In embodiments, the ligand binding domain includes an amino acid sequence of about 1 to about 450 amino acids in length. In embodiments, the ligand binding domain includes an amino acid sequence of about 1 to about 400 amino acids in length. In embodiments, the ligand binding domain includes an amino acid sequence of about 1 to about 350 amino acids in length. In embodiments, the ligand binding domain includes an amino acid sequence of about 1 to about 300 amino acids in length. In embodiments, the ligand binding domain includes an amino acid sequence of about 1 to about 250 amino acids in length. In embodiments, the ligand binding domain includes an amino acid sequence of about 1 to about 200 amino acids in length. In embodiments, the ligand binding domain includes an amino acid sequence of about 1 to about 150 amino acids in length. In embodiments, the ligand binding domain includes an amino acid sequence of about 1 to about 100 amino acids in length. In embodiments, the ligand binding domain includes an amino acid sequence of about 1 to about 75 amino acids in length. In embodiments, the ligand binding domain includes an amino acid sequence of about 1 to about 50 amino acids in length. In embodiments, the ligand binding domain includes an amino acid sequence of about 1 to about 25 amino acids in length. In embodiments, the ligand binding domain includes an amino acid sequence of about 1 to about 20 amino acids in length. In embodiments, the ligand binding domain includes an amino acid sequence of about 1 to about 15 amino acids in length. In embodiments, the ligand binding domain includes an amino acid sequence of about 1 to about 10 amino acids in length. In embodiments, the ligand binding domain includes an amino acid sequence of about 1 to about 9 amino acids in length. In embodiments, the ligand binding domain includes an amino acid sequence of about 1 to about 8 amino acids in length. In embodiments, the ligand binding domain includes an amino acid sequence of about 1 to about 7 amino acids in length. In embodiments, the ligand binding domain includes an amino acid sequence of about 1 to about 6 amino acids in length. In embodiments, the ligand binding domain includes an amino acid sequence of about 1 to about 5 amino acids in length. In embodiments, the ligand binding domain includes an amino acid sequence of about 1 to about 4 amino acids in length. In embodiments, the ligand binding domain includes an amino acid sequence of about 1 to about 3 amino acids in length. In embodiments, the ligand binding domain includes an amino acid sequence of about 1 to about 2 amino acids in length.

In embodiments, the ligand binding domain includes an amino acid sequence of 1 to 500 amino acids in length. In embodiments, the ligand binding domain includes an amino acid sequence of 2 to 500 amino acids in length. In embodiments, the ligand binding domain includes an amino acid sequence of 3 to 500 amino acids in length. In embodiments, the ligand binding domain includes an amino acid sequence of 4 to 500 amino acids in length. In embodiments, the ligand binding domain includes an amino acid sequence of 5 to 500 amino acids in length. In embodiments, the ligand binding domain includes an amino acid sequence of 6 to 500 amino acids in length. In embodiments, the ligand binding domain includes an amino acid sequence of 7 to 500 amino acids in length. In embodiments, the ligand binding domain includes an amino acid sequence of 8 to 500 amino acids in length. In embodiments, the ligand binding domain includes an amino acid sequence of 9 to 500 amino acids in length. In embodiments, the ligand binding domain includes an amino acid sequence of 10 to 500 amino acids in length. In embodiments, the ligand binding domain includes an amino acid sequence of 15 to 500 amino acids in length. In embodiments, the ligand binding domain includes an amino acid sequence of 20 to 500 amino acids in length. In embodiments, the ligand binding domain includes an amino acid sequence of 25 to 500 amino acids in length. In embodiments, the ligand binding domain includes an amino acid sequence of 50 to 500 amino acids in length. In embodiments, the ligand binding domain includes an amino acid sequence of 75 to 500 amino acids in length. In embodiments, the ligand binding domain includes an amino acid sequence of 100 to 500 amino acids in length. In embodiments, the ligand binding domain includes an amino acid sequence of 150 to 500 amino acids in length. In embodiments, the ligand binding domain includes an amino acid sequence of 200 to 500 amino acids in length. In embodiments, the ligand binding domain includes an amino acid sequence of 250 to 500 amino acids in length. In embodiments, the ligand binding domain includes an amino acid sequence of 300 to 500 amino acids in length. In embodiments, the ligand binding domain includes an amino acid sequence of 350 to 500 amino acids in length. In embodiments, the ligand binding domain includes an amino acid sequence of 400 to 500 amino acids in length. In embodiments, the ligand binding domain includes an amino acid sequence of 450 to 500 amino acids in length.

In embodiments, the ligand binding domain includes an amino acid sequence of 1 to 450 amino acids in length. In embodiments, the ligand binding domain includes an amino acid sequence of 1 to 400 amino acids in length. In embodiments, the ligand binding domain includes an amino acid sequence of 1 to 350 amino acids in length. In embodiments, the ligand binding domain includes an amino acid sequence of 1 to 300 amino acids in length. In embodiments, the ligand binding domain includes an amino acid sequence of 1 to 250 amino acids in length. In embodiments, the ligand binding domain includes an amino acid sequence of 1 to 200 amino acids in length. In embodiments, the ligand binding domain includes an amino acid sequence of 1 to 150 amino acids in length. In embodiments, the ligand binding domain includes an amino acid sequence of 1 to 100 amino acids in length. In embodiments, the ligand binding domain includes an amino acid sequence of 1 to 75 amino acids in length. In embodiments, the ligand binding domain includes an amino acid sequence of 1 to 50 amino acids in length. In embodiments, the ligand binding domain includes an amino acid sequence of 1 to 25 amino acids in length. In embodiments, the ligand binding domain includes an amino acid sequence of 1 to 20 amino acids in length. In embodiments, the ligand binding domain includes an amino acid sequence of 1 to 15 amino acids in length. In embodiments, the ligand binding domain includes an amino acid sequence of 1 to 10 amino acids in length. In embodiments, the ligand binding domain includes an amino acid sequence of 1 to 9 amino acids in length. In embodiments, the ligand binding domain includes an amino acid sequence of 1 to 8 amino acids in length. In embodiments, the ligand binding domain includes an amino acid sequence of 1 to 7 amino acids in length. In embodiments, the ligand binding domain includes an amino acid sequence of 1 to 6 amino acids in length. In embodiments, the ligand binding domain includes an amino acid sequence of 1 to 5 amino acids in length. In embodiments, the ligand binding domain includes an amino acid sequence of 1 to 4 amino acids in length. In embodiments, the ligand binding domain includes an amino acid sequence of 1 to 3 amino acids in length. In embodiments, the ligand binding domain includes an amino acid sequence of 1 to 2 amino acids in length.

In embodiments, the ligand binding domain is an antibody domain. In embodiments, the ligand binding domain is an Fc domain. In embodiments, the ligand binding domain includes the amino acid sequence of SEQ ID NO:3. In embodiments, the ligand binding domain is the amino acid sequence of SEQ ID NO:3.

The chimeric binding peptide may include a polyhistidine domain and be referred to as His-tagged. The polyhistidine domain or His-tag may bind a metal ion. The binding of a polyhistidine domain to a metal ion may be used to isolate CAR-expressing T-cells. In embodiments, the ligand binding domain is a polyhistidine domain. In further embodiments, the polyhistidine domain includes an amino acid sequence including 1 or more (e.g. 2, 3, 4, 5, 6, etc.) histidine amino acids. In embodiments, the polyhistine domain is an amino acid sequence that is 1 or more (e.g. 2, 3, 4, 5, 6, etc.) histidine amino acids. In embodiments, the ligand is a metal ion. In further embodiments, the metal ion is a cobalt ion, a copper ion, or a nickel ion. In embodiments, the metal ion is a cobalt ion. In embodiments, the metal ion is a copper ion. In embodiments, the metal ion is a nickel ion. In embodiments, the metal ion is bound to a particle or a solid support. In embodiments, the metal ion is bound to a particle. In embodiments, the metal ion is bound to a solid support. In embodiments, the polyhistidine domain binds a plurality (e.g. more than 2) of metal ions. In embodiments, the plurality of metal ions is bound to one or more particles or a solid support.

In embodiments, the detectable moiety is a fluorescent moiety. In embodiments, the detectable moiety is a phycoerythrin (PE) moiety, a fluorescein isothiocynate (FITC) moiety, or an Alexa Fluor 647 moiety. In embodiments, the detectable moiety is a phycoerythrin (PE) moiety. In embodiments, the detectable moiety is a fluorescein isothiocynate (FITC) moiety. In embodiments, the detectable moiety is an Alexa Fluor 647 moiety.

In embodiments, the chimeric binding peptide comprises a ROR1 domain, an Fc domain and one or more PE moieties. In embodiments, the chimeric binding peptide includes the amino acid sequence of SEQ ID NO:4. In embodiments, the chimeric binding peptide is the amino acid sequence of SEQ ID NO:4.

In embodiments, the chimeric binding peptide is non-immunogenic. In embodiments, the chimeric binding peptide includes a linear structure. In embodiments, the chimeric binding peptide is linear. In embodiments, the chimeric binding peptide does not include a globular structure. In embodiments, the chimeric binding peptide is not globular.

In embodiments, the chimeric binding peptide includes a ROR1 CAR-binding domain, a Fc ligand binding domain and a plurality of PE detectable moieties attached to the CAR binding domain and the Fc ligand binding domain. In embodiments, the chimeric binding peptide includes the amino acid sequence of SEQ ID NO:4 and a plurality of PE detectable moieties bound thereto. In embodiments, the chimeric binding peptide is the amino acid sequence of SEQ ID NO:4 and a plurality of PE detectable moieties bound thereto. In one further embodiment, the chimeric binding peptide is referred to herein as ROR-Ig

In one embodiment, the method of isolating a chimeric antigen receptor (CAR)-expressing T-cell, includes: (i) contacting a cell population including a ROR1 CAR-expressing T-cell with a ROR-Ig chimeric binding peptide, thereby forming a ROR-Ig chimeric peptide-ROR1 CAR T-cell complex; (ii) contacting the ROR-Ig chimeric peptide-ROR1 CAR T-cell complex with an anti-PE antibody-coated magnetic bead, thereby forming an anti-PE magnetic bead-bound ROR-Ig chimeric peptide-ROR1 CAR T-cell complex; (iii) separating the anti-PE magnetic bead-bound ROR-Ig chimeric peptide-ROR1 CAR T-cell complex from the cell population, thereby isolating a ROR1 CAR T-cell population; and (iv) expanding the isolated ROR1 CAR T-cell population to form a purified ROR1 CAR T-cell population.

### Chimeric Peptide-CAR T-Cell Complexes

Provided herein, inter alia, are chimeric peptide-chimeric antigen receptor (CAR) T-cell complexes useful for the isolation and purification of a population of CAR-expressing T-cells. Thus, in an aspect is provided a chimeric peptide-chimeric antigen receptor (CAR) T-cell complex including: a CAR-expressing T-cell bound to a chimeric binding peptide, the chimeric binding peptide including a CAR-binding domain a ligand binding domain, and a detectable moiety. In embodiments, the CAR-binding domain is bound to the CAR. In embodiments, the binding of the CAR-binding domain does not activate a CAR T-cell at a detectable level.

In embodiments, the CAR-expressing T-cell is a ROR1 CAR T-cell, a ROR2 CAR T-cell, or a CD19 CAR T-cell. In embodiments, the CAR-expressing T-cell is a ROR1 CAR T-cell. In embodiments, the CAR-expressing T-cell is a ROR2 CAR T-cell. In embodiments, the CAR-expressing T-cell is a CD19 CAR T-cell.

In embodiments, the CAR-expressing T-cell is a TNFRSF17 CAR T-cell, a TNFRSF8 CAR T-cell, a CD5 CAR T-cell, a ULBP1 CAR T-cell, a ULBP2 CAR T-cell, a CEACAM5 CAR T-cell, a CD276 CAR T-cell, an IL3RA CAR T-cell, a MS4A1 CAR T-cell, a CD33 CAR T-cell, a NCAM1 CAR T-cell, a MET CAR T-cell, a CSPG4 CAR T-cell, a SDC1 CAR T-cell, a CD22 CAR T-cell, a CD38 CAR T-cell, a CD70 CAR T-cell, an IL1RAP CAR T-cell, an EGFR CAR T-cell, a CD133 CAR T-cell, an EGFRVIII CAR T-cell, an EPCAM CAR T-cell, an EPHA2 CAR T-cell, an ERBB2 CAR T-cell, a GPC3 CAR T-cell, a MSLN CAR T-cell, a TEM1 CAR T-cell, a MUC1 CAR T-cell, a PDCD1 CAR T-cell, a CD274 CAR T-cell, a KDR CAR T-cell, an IL13RA2 CAR T-cell, a FOLH1 CAR T-cell, a GPNMB CAR T-cell, a FAP CAR T-cell, a CA9 CAR T-cell, a FOLR1 CAR T-cell, a L1CAM CAR T-cell, a CD23 CAR T-cell, a PSCA CAR T-cell, a CD44 CAR T-cell, a CD174 CAR T-cell, a SLAMF7 CAR T-cell, a GD2 CAR T-cell, a BCMA CAR T-cell, a CD30 CAR T-cell, a CD4 CAR T-cell, a CD7 CAR T-cell, a CD79B CAR T-cell, a CLEC12A CAR T-cell, a Siglec-2 CAR T-cell, a Siglec-3 CAR T-cell, a SIRP alpha CAR T-cell, a uPAR CAR T-cell, a VEGFR2 CAR T-cell, a GUCY2C CAR T-cell, or a Protein L CAR T-cell.

In embodiments, the CAR-expressing T-cell is a TNFRSF17 CAR T-cell. In embodiments, the CAR-expressing T-cell is a TNFRSF8 CAR T-cell. In embodiments, the CAR-expressing T-cell is a CD5 CAR T-cell. In embodiments, the CAR-expressing T-cell is a ULBP1 CAR T-cell. In embodiments, the CAR-expressing T-cell is a ULBP2 CAR T-cell. In embodiments, the CAR-expressing T-cell is a CEACAM5 CAR T-cell. In embodiments, the CAR-expressing T-cell is a CD276 CAR T-cell. In embodiments, the CAR-expressing T-cell is an IL3RA CAR T-cell. In embodiments, the CAR-expressing T-cell is a MS4A1 CAR T-cell. In embodiments, the CAR-expressing T-cell is a CD33 CAR T-cell. In embodiments, the CAR-expressing T-cell is a NCAM1 CAR T-cell. In embodiments, the CAR-expressing T-cell is a MET CAR T-cell. In embodiments, the CAR-expressing T-cell is a CSPG4 CAR T-cell. In embodiments, the CAR-expressing T-cell is a SDC1 CAR T-cell. In embodiments, the CAR-expressing T-cell is a CD22 CAR T-cell. In embodiments, the CAR-expressing T-cell is a CD38 CAR T-cell. In embodiments, the CAR-expressing T-cell is a CD70 CAR T-cell. In embodiments, the CAR-expressing T-cell is an IL1RAP CAR T-cell. In embodiments, the CAR-expressing T-cell is an EGFR CAR T-cell. In embodiments, the CAR-expressing T-cell is a CD133 CAR T-cell. In embodiments, the CAR-expressing T-cell is an EGFRVIII CAR T-cell. In embodiments, the CAR-expressing T-cell is an EPCAM CAR T-cell. In embodiments, the CAR-expressing T-cell is an EPHA2 CAR T-cell. In embodiments, the CAR-expressing T-cell is an ERBB2 CAR T-cell. In embodiments, the CAR-expressing T-cell is a GPC3 CAR T-cell. In embodiments, the CAR-expressing T-cell is a MSLN CAR T-cell. In embodiments, the CAR-expressing T-cell is a TEM1 CAR T-cell. In embodiments, the CAR-expressing T-cell is a MUC1 CAR T-cell. In embodiments, the CAR-expressing T-cell is a PDCD1 CAR T-cell. In embodiments, the CAR-expressing T-cell is a CD274 CAR T-cell. In embodiments, the CAR-expressing T-cell is a KDR CAR T-cell. In embodiments, the CAR-expressing T-cell is an IL13RA2 CAR T-cell. In embodiments, the CAR-expressing T-cell is a FOLH1 CAR T-cell. In embodiments, the CAR-expressing T-cell is a GPNMB CAR T-cell. In embodiments, the CAR-expressing T-cell is a FAP CAR T-cell. In embodiments, the CAR-expressing T-cell is a CA9 CAR T-cell. In embodiments, the CAR-expressing T-cell is a FOLR1 CAR T-cell. In embodiments, the CAR-expressing T-cell is a L1CAM CAR T-cell, a CD23 CAR T-cell. In embodiments, the CAR-expressing T-cell is a PSCA CAR T-cell. In embodiments, the CAR-expressing T-cell is a CD44 CAR T-cell. In embodiments, the CAR-expressing T-cell is a CD174 CAR T-cell. In embodiments, the CAR-expressing T-cell is a SLAMF7 CAR T-cell. In embodiments, the CAR-expressing T-cell is a GD2 CAR T-cell. In embodiments, the CAR-expressing T-cell is a BCMA CAR T-cell. In embodiments, the CAR-expressing T-cell is a CD30 CAR T-cell. In embodiments, the CAR-expressing T-cell is a CD4 CAR T-cell. In embodiments, the CAR-expressing T-cell is a CD7 CAR T-cell. In embodiments, the CAR-expressing T-cell is a CD79B CAR T-cell. In embodiments, the CAR-expressing T-cell is a CLEC12A CAR T-cell. In embodiments, the CAR-expressing T-cell is a Siglec-2 CAR T-cell. In embodiments, the CAR-expressing T-cell is a Siglec-3 CAR T-cell. In embodiments, the CAR-expressing T-cell is a SIRP alpha CAR T-cell. In embodiments, the CAR-expressing T-cell is a uPAR CAR T-cell, a VEGFR2 CAR T-cell. In embodiments, the CAR-expressing T-cell is a GUCY2C CAR T-cell. In embodiments, the CAR-expressing T-cell is a Protein L CAR T-cell.

In embodiments, the complex further includes a ligand bound to the ligand binding domain. In embodiments, the binding is covalent or non-covalent. In embodiments, the binding is covalent. In embodiments, the binding is non-covalent.

In embodiments, the ligand is a protein, a nucleic acid, or a small molecule. In embodiments, the ligand is a protein. In embodiments, the ligand is a nucleic acid. In embodiments, the ligand is a small molecule. In embodiments, the ligand is an antibody domain.

In embodiments, the complex is bound to a metal-coated particle or a solid support through the ligand. In embodiments, the complex is bound to a metal-coated particle. In embodiments, the complex is bound to a solid support.

In embodiments, the CAR-binding domain and the ligand binding domain are connected through a chemical linker. In embodiments, the chemical linker is a peptide.

In embodiments, the chemical linker includes an amino acid sequence of about 2 to about 25 amino acids in length. In embodiments, the chemical linker includes an amino acid sequence of about 3 to about 25 amino acids in length. In embodiments, the chemical linker includes an amino acid sequence of about 4 to about 25 amino acids in length. In embodiments, the chemical linker includes an amino acid sequence of about 5 to about 25 amino acids in length. In embodiments, the chemical linker includes an amino acid sequence of about 6 to about 25 amino acids in length. In embodiments, the chemical linker includes an amino acid sequence of about 7 to about 25 amino acids in length. In embodiments, the chemical linker includes an amino acid sequence of about 8 to about 25 amino acids in length. In embodiments, the chemical linker includes an amino acid sequence of about 9 to about 25 amino acids in length. In embodiments, the chemical linker includes an amino acid sequence of about 10 to about 25 amino acids in length. In embodiments, the chemical linker includes an amino acid sequence of about 11 to about 25 amino acids in length. In embodiments, the chemical linker includes an amino acid sequence of about 12 to about 25 amino acids in length. In embodiments, the chemical linker includes an amino acid sequence of about 13 to about 25 amino acids in length. In embodiments, the chemical linker includes an amino acid sequence of about 14 to about 25 amino acids in length. In embodiments, the chemical linker includes an amino acid sequence of about 15 to about 25 amino acids in length. In embodiments, the chemical linker includes an amino acid sequence of about 16 to about 25 amino acids in length. In embodiments, the chemical linker includes an amino acid sequence of about 17 to about 25 amino acids in length. In embodiments, the chemical linker includes an amino acid sequence of about 18 to about 25 amino acids in length. In embodiments, the chemical linker includes an amino acid sequence of about 19 to about 25 amino acids in length. In embodiments, the chemical linker includes an amino acid sequence of about 20 to about 25 amino acids in length. In embodiments, the chemical linker includes an amino acid sequence of about 21 to about 25 amino acids in length. In embodiments, the chemical linker includes an amino acid sequence of about 22 to about 25 amino acids in length. In embodiments, the chemical linker includes an amino acid sequence of about 23 to about 25 amino acids in length. In embodiments, the chemical linker includes an amino acid sequence of about 24 to about 25 amino acids in length.

In embodiments, the chemical linker includes an amino acid sequence of about 2 to about 24 amino acids in length. In embodiments, the chemical linker includes an amino acid sequence of about 2 to about 23 amino acids in length. In embodiments, the chemical linker includes an amino acid sequence of about 2 to about 22 amino acids in length. In embodiments, the chemical linker includes an amino acid sequence of about 2 to about 21 amino acids in length. In embodiments, the chemical linker includes an amino acid sequence of about 2 to about 20 amino acids in length. In embodiments, the chemical linker includes an amino acid sequence of about 2 to about 19 amino acids in length. In embodiments, the chemical linker includes an amino acid sequence of about 2 to about 18 amino acids in length. In embodiments, the chemical linker includes an amino acid sequence of about 2 to about 17 amino acids in length. In embodiments, the chemical linker includes an amino acid sequence of about 2 to about 16 amino acids in length. In embodiments, the chemical linker includes an amino acid sequence of about 2 to about 15 amino acids in length. In embodiments, the chemical linker includes an amino acid sequence of about 2 to about 14 amino acids in length. In embodiments, the chemical linker includes an amino acid sequence of about 2 to about 13 amino acids in length. In embodiments, the chemical linker includes an amino acid sequence of about 2 to about 12 amino acids in length. In embodiments, the chemical linker includes an amino acid sequence of about 2 to about 11 amino acids in length. In embodiments, the chemical linker includes an amino acid sequence of about 2 to about 10 amino acids in length. In embodiments, the chemical linker includes an amino acid sequence of about 2 to about 9 amino acids in length. In embodiments, the chemical linker includes an amino acid sequence of about 2 to about 8 amino acids in length. In embodiments, the chemical linker includes an amino acid sequence of about 2 to about 7 amino acids in length. In embodiments, the chemical linker includes an amino acid sequence of about 2 to about 6 amino acids in length. In embodiments, the chemical linker includes an amino acid sequence of about 2 to about 5 amino acids in length. In embodiments, the chemical linker includes an amino acid sequence of about 2 about 4 amino acids in length. In embodiments, the chemical linker includes an amino acid sequence of about 2 to about 3 amino acids in length.

In embodiments, the chemical linker is an amino acid sequence of 2 to 25 amino acids in length. In embodiments, the chemical linker is an amino acid sequence of 3 to 25 amino acids in length. In embodiments, the chemical linker is an amino acid sequence of 4 to 25 amino acids in length. In embodiments, the chemical linker is an amino acid sequence of 5 to 25 amino acids in length. In embodiments, the chemical linker is an amino acid sequence of 6 to 25 amino acids in length. In embodiments, the chemical linker is an amino acid sequence of 7 to 25 amino acids in length. In embodiments, the chemical linker is an amino acid sequence of 8 to 25 amino acids in length. In embodiments, the chemical linker is an amino acid sequence of 9 to 25 amino acids in length. In embodiments, the chemical linker is an amino acid sequence of 10 to 25 amino acids in length. In embodiments, the chemical linker is an amino acid sequence of 11 to 25 amino acids in length. In embodiments, the chemical linker is an amino acid sequence of 12 to 25 amino acids in length. In embodiments, the chemical linker is an amino acid sequence of 13 to 25 amino acids in length. In embodiments, the chemical linker is an amino acid sequence of 14 to 25 amino acids in length. In embodiments, the chemical linker is an amino acid sequence of 15 to 25 amino acids in length. In embodiments, the chemical linker is an amino acid sequence of 16 to 25 amino acids in length. In embodiments, the chemical linker is an amino acid sequence of 17 to 25 amino acids in length. In embodiments, the chemical linker is an amino acid sequence of 18 to 25 amino acids in length. In embodiments, the chemical linker is an amino acid sequence of 19 to 25 amino acids in length. In embodiments, the chemical linker is an amino acid sequence of 20 to 25 amino acids in length. In embodiments, the chemical linker is an amino acid sequence of 21 to 25 amino acids in length. In embodiments, the chemical linker is an amino acid sequence of 22 to 25 amino acids in length. In embodiments, the chemical linker is an amino acid sequence of 23 to 25 amino acids in length. In embodiments, the chemical linker is an amino acid sequence of 24 to 25 amino acids in length.

In embodiments, the chemical linker includes an amino acid sequence of 2 to 24 amino acids in length. In embodiments, the chemical linker includes an amino acid sequence of 2 to 23 amino acids in length. In embodiments, the chemical linker includes an amino acid sequence of 2 to 22 amino acids in length. In embodiments, the chemical linker includes an amino acid sequence of 2 to 21 amino acids in length. In embodiments, the chemical linker includes an amino acid sequence of 2 to 20 amino acids in length. In embodiments, the chemical linker includes an amino acid sequence of 2 to 19 amino acids in length. In embodiments, the chemical linker includes an amino acid sequence of 2 to 18 amino acids in length. In embodiments, the chemical linker includes an amino acid sequence of 2 to 17 amino acids in length. In embodiments, the chemical linker includes an amino acid sequence of 2 to 16 amino acids in length. In embodiments, the chemical linker includes an amino acid sequence of 2 to 15 amino acids in length. In embodiments, the chemical linker includes an amino acid sequence of 2 to 14 amino acids in length. In embodiments, the chemical linker includes an amino acid sequence of 2 to 13 amino acids in length. In embodiments, the chemical linker includes an amino acid sequence of 2 to 12 amino acids in length. In embodiments, the chemical linker includes an amino acid sequence of 2 to 11 amino acids in length. In embodiments, the chemical linker includes an amino acid sequence of 2 to 10 amino acids in length. In embodiments, the chemical linker includes an amino acid sequence of 2 to 9 amino acids in length. In embodiments, the chemical linker includes an amino acid sequence of 2 to 8 amino acids in length. In embodiments, the chemical linker includes an amino acid sequence of 2 to 7 amino acids in length. In embodiments, the chemical linker includes an amino acid sequence of 2 to 6 amino acids in length. In embodiments, the chemical linker includes an amino acid sequence of 2 to 5 amino acids in length. In embodiments, the chemical linker includes an amino acid sequence of 2 to 4 amino acids in length. In embodiments, the chemical linker includes an amino acid sequence of 2 to 3 amino acids in length.

In embodiments, the chemical linker includes the amino acid sequence of SEQ ID NO: 2. In embodiments, the chemical linker is the amino acid sequence of SEQ ID NO:2.

In embodiments, the chimeric binding peptide includes an amino acid sequence of about 25 to about 1000 amino acids in length. In embodiments, the chimeric binding peptide includes an amino acid sequence of about 50 to about 1000 amino acids in length. In embodiments, the chimeric binding peptide includes an amino acid sequence of about 75 to about 1000 amino acids in length. In embodiments, the chimeric binding peptide includes an amino acid sequence of about 100 to about 1000 amino acids in length. In embodiments, the chimeric binding peptide includes an amino acid sequence of about 150 to about 1000 amino acids in length. In embodiments, the chimeric binding peptide includes an amino acid sequence of about 200 to about 1000 amino acids in length. In embodiments, the chimeric binding peptide includes an amino acid sequence of about 250 to about 1000 amino acids in length. In embodiments, the chimeric binding peptide includes an amino acid sequence of about 300 to about 1000 amino acids in length. In embodiments, the chimeric binding peptide includes an amino acid sequence of about 350 to about 1000 amino acids in length. In embodiments, the chimeric binding peptide includes an amino acid sequence of about 400 to about 1000 amino acids in length. In embodiments, the chimeric binding peptide includes an amino acid sequence of about 450 to about 1000 amino acids in length. In embodiments, the chimeric binding peptide includes an amino acid sequence of about 500 to about 1000 amino acids in length. In embodiments, the chimeric binding peptide includes an amino acid sequence of about 550 to about 1000 amino acids in length. In embodiments, the chimeric binding peptide includes an amino acid sequence of about 600 to about 1000 amino acids in length. In embodiments, the chimeric binding peptide includes an amino acid sequence of about 650 to about 1000 amino acids in length. In embodiments, the chimeric binding peptide includes an amino acid sequence of about 700 to about 1000 amino acids in length. In embodiments, the chimeric binding peptide includes an amino acid sequence of about 750 to about 1000 amino acids in length. In embodiments, the chimeric binding peptide includes an amino acid sequence of about 800 to about 1000 amino acids in length. In embodiments, the chimeric binding peptide includes an amino acid sequence of about 850 to about 1000 amino acids in length. In embodiments, the chimeric binding peptide includes an amino acid sequence of about 900 to about 1000 amino acids in length. In embodiments, the chimeric binding peptide includes an amino acid sequence of about 950 to about 1000 amino acids in length.

In embodiments, the chimeric binding peptide includes an amino acid sequence of about 25 to about 950 amino acids in length. In embodiments, the chimeric binding peptide includes an amino acid sequence of about 25 to about 900 amino acids in length. In embodiments, the chimeric binding peptide includes an amino acid sequence of about 25 to about 850 amino acids in length. In embodiments, the chimeric binding peptide includes an amino acid sequence of about 25 to about 800 amino acids in length. In embodiments, the chimeric binding peptide includes an amino acid sequence of about 25 to about 750 amino acids in length. In embodiments, the chimeric binding peptide includes an amino acid sequence of about 25 to about 700 amino acids in length. In embodiments, the chimeric binding peptide includes an amino acid sequence of about 25 to about 650 amino acids in length. In embodiments, the chimeric binding peptide includes an amino acid sequence of about 25 to about 600 amino acids in length. In embodiments, the chimeric binding peptide includes an amino acid sequence of about 25 to about 550 amino acids in length. In embodiments, the chimeric binding peptide includes an amino acid sequence of about 25 to about 500 amino acids in length. In embodiments, the chimeric binding peptide includes an amino acid sequence of about 25 to about 450 amino acids in length. In embodiments, the chimeric binding peptide includes an amino acid sequence of about 25 to about 400 amino acids in length. In embodiments, the chimeric binding peptide includes an amino acid sequence of about 25 to about 350 amino acids in length. In embodiments, the chimeric binding peptide includes an amino acid sequence of about 25 to about 300 amino acids in length. In embodiments, the chimeric binding peptide includes an amino acid sequence of about 25 to about 250 amino acids in length. In embodiments, the chimeric binding peptide includes an amino acid sequence of about 25 to about 200 amino acids in length. In embodiments, the chimeric binding peptide includes an amino acid sequence of about 25 to about 150 amino acids in length. In embodiments, the chimeric binding peptide includes an amino acid sequence of about 25 to about 100 amino acids in length. In embodiments, the chimeric binding peptide includes an amino acid sequence of about 25 to about 75 amino acids in length. In embodiments, the chimeric binding peptide includes an amino acid sequence of about 25 to about 50 amino acids in length.

In embodiments, the chimeric binding peptide includes an amino acid sequence of 25 to 1000 amino acids in length. In embodiments, the chimeric binding peptide includes an amino acid sequence of 50 to 1000 amino acids in length. In embodiments, the chimeric binding peptide includes an amino acid sequence of 75 to 1000 amino acids in length. In embodiments, the chimeric binding peptide includes an amino acid sequence of 100 to 1000 amino acids in length. In embodiments, the chimeric binding peptide includes an amino acid sequence of 150 to 1000 amino acids in length. In embodiments, the chimeric binding peptide includes an amino acid sequence of 200 to 1000 amino acids in length. In embodiments, the chimeric binding peptide includes an amino acid sequence of 250 to 1000 amino acids in length. In embodiments, the chimeric binding peptide includes an amino acid sequence of 300 to 1000 amino acids in length. In embodiments, the chimeric binding peptide includes an amino acid sequence of 350 to 1000 amino acids in length. In embodiments, the chimeric binding peptide includes an amino acid sequence of 400 to 1000 amino acids in length. In embodiments, the chimeric binding peptide includes an amino acid sequence of 450 to 1000 amino acids in length. In embodiments, the chimeric binding peptide includes an amino acid sequence of 500 to 1000 amino acids in length. In embodiments, the chimeric binding peptide includes an amino acid sequence of 550 to 1000 amino acids in length. In embodiments, the chimeric binding peptide includes an amino acid sequence of 600 to 1000 amino acids in length. In embodiments, the chimeric binding peptide includes an amino acid sequence of 650 to 1000 amino acids in length. In embodiments, the chimeric binding peptide includes an amino acid sequence of 700 to 1000 amino acids in length. In embodiments, the chimeric binding peptide includes an amino acid sequence of 750 to 1000 amino acids in length. In embodiments, the chimeric binding peptide includes an amino acid sequence of 800 to 1000 amino acids in length. In embodiments, the chimeric binding peptide includes an amino acid sequence of 850 to 1000 amino acids in length. In embodiments, the chimeric binding peptide includes an amino acid sequence of 900 to 1000 amino acids in length. In embodiments, the chimeric binding peptide includes an amino acid sequence of 950 to 1000 amino acids in length.

In embodiments, the chimeric binding peptide includes an amino acid sequence of 25 to 950 amino acids in length. In embodiments, the chimeric binding peptide includes an amino acid sequence of 25 to 900 amino acids in length. In embodiments, the chimeric binding peptide includes an amino acid sequence of 25 to 850 amino acids in length. In embodiments, the chimeric binding peptide includes an amino acid sequence of 25 to 800 amino acids in length. In embodiments, the chimeric binding peptide includes an amino acid sequence of 25 to 750 amino acids in length. In embodiments, the chimeric binding peptide includes an amino acid sequence of 25 to 700 amino acids in length. In embodiments, the chimeric binding peptide includes an amino acid sequence of 25 to 650 amino acids in length. In embodiments, the chimeric binding peptide includes an amino acid sequence of 25 to 600 amino acids in length. In embodiments, the chimeric binding peptide includes an amino acid sequence of 25 to 550 amino acids in length. In embodiments, the chimeric binding peptide includes an amino acid sequence of 25 to 500 amino acids in length. In embodiments, the chimeric binding peptide includes an amino acid sequence of 25 to 450 amino acids in length. In embodiments, the chimeric binding peptide includes an amino acid sequence of 25 to 400 amino acids in length. In embodiments, the chimeric binding peptide includes an amino acid sequence of 25 to 350 amino acids in length. In embodiments, the chimeric binding peptide includes an amino acid sequence of 25 to 300 amino acids in length. In embodiments, the chimeric binding peptide includes an amino acid sequence of 25 to 250 amino acids in length. In embodiments, the chimeric binding peptide includes an amino acid sequence of 25 to 200 amino acids in length. In embodiments, the chimeric binding peptide includes an amino acid sequence of 25 to 150 amino acids in length. In embodiments, the chimeric binding peptide includes an amino acid sequence of 25 to 100 amino acids in length. In embodiments, the chimeric binding peptide includes an amino acid sequence of 25 to 75 amino acids in length. In embodiments, the chimeric binding peptide includes an amino acid sequence of 25 to 50 amino acids in length.

In embodiments, CAR-binding domain includes an amino acid sequence of about 25 to about 500 amino acids in length. In embodiments, CAR-binding domain includes an amino acid sequence of about 50 to about 500 amino acids in length. In embodiments, CAR-binding domain includes an amino acid sequence of about 75 to about 500 amino acids in length. In embodiments, CAR-binding domain includes an amino acid sequence of about 100 to about 500 amino acids in length. In embodiments, CAR-binding domain includes an amino acid sequence of about 150 to about 500 amino acids in length. In embodiments, CAR-binding domain includes an amino acid sequence of about 200 to about 500 amino acids in length. In embodiments, CAR-binding domain includes an amino acid sequence of about 250 to about 500 amino acids in length. In embodiments, CAR-binding domain includes an amino acid sequence of about 300 to about 500 amino acids in length. In embodiments, CAR-binding domain includes an amino acid sequence of about 350 to about 500 amino acids in length. In embodiments, CAR-binding domain includes an amino acid sequence of about 400 to about 500 amino acids in length. In embodiments, CAR-binding domain includes an amino acid sequence of about 450 to about 500 amino acids in length.

In embodiments, CAR-binding domain includes an amino acid sequence of about 25 to about 450 amino acids in length. In embodiments, CAR-binding domain includes an amino acid sequence of about 25 to about 400 amino acids in length. In embodiments, CAR-binding domain includes an amino acid sequence of about 25 to about 350 amino acids in length. In embodiments, CAR-binding domain includes an amino acid sequence of about 25 to about 300 amino acids in length. In embodiments, CAR-binding domain includes an amino acid sequence of about 25 to about 250 amino acids in length. In embodiments, CAR-binding domain includes an amino acid sequence of about 25 to about 200 amino acids in length. In embodiments, CAR-binding domain includes an amino acid sequence of about 25 to about 150 amino acids in length. In embodiments, CAR-binding domain includes an amino acid sequence of about 25 to about 100 amino acids in length. In embodiments, CAR-binding domain includes an amino acid sequence of about 25 to about 75 amino acids in length. In embodiments, CAR-binding domain includes an amino acid sequence of about 25 to about 50 amino acids in length.

In embodiments, CAR-binding domain includes an amino acid sequence of 25 to 500 amino acids in length. In embodiments, CAR-binding domain includes an amino acid sequence of 50 to 500 amino acids in length. In embodiments, CAR-binding domain includes an amino acid sequence of 75 to 500 amino acids in length. In embodiments, CAR-binding domain includes an amino acid sequence of 100 to 500 amino acids in length. In embodiments, CAR-binding domain includes an amino acid sequence of 150 to 500 amino acids in length. In embodiments, CAR-binding domain includes an amino acid sequence of 200 to 500 amino acids in length. In embodiments, CAR-binding domain includes an amino acid sequence of 250 to 500 amino acids in length. In embodiments, CAR-binding domain includes an amino acid sequence of 300 to 500 amino acids in length. In embodiments, CAR-binding domain includes an amino acid sequence of 350 to 500 amino acids in length. In embodiments, CAR-binding domain includes an amino acid sequence of 400 to 500 amino acids in length. In embodiments, CAR-binding domain includes an amino acid sequence of 450 to 500 amino acids in length.

In embodiments, CAR-binding domain includes an amino acid sequence of 25 to 450 amino acids in length. In embodiments, CAR-binding domain includes an amino acid sequence of 25 to 400 amino acids in length. In embodiments, CAR-binding domain includes an amino acid sequence of 25 to 350 amino acids in length. In embodiments, CAR-binding domain includes an amino acid sequence of 25 to 300 amino acids in length. In embodiments, CAR-binding domain includes an amino acid sequence of 25 to 250 amino acids in length. In embodiments, CAR-binding domain includes an amino acid sequence of 25 to 200 amino acids in length. In embodiments, CAR-binding domain includes an amino acid sequence of 25 to 150 amino acids in length. In embodiments, CAR-binding domain includes an amino acid sequence of 25 to 100 amino acids in length. In embodiments, CAR-binding domain includes an amino acid sequence of 25 to 75 amino acids in length. In embodiments, CAR-binding domain includes an amino acid sequence of 25 to 50 amino acids in length.

In embodiments, the CAR-binding domain is a cancer antigen or fragment thereof. In embodiments, the CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme. In embodiments, the CAR-binding domain is a receptor tyrosine kinase domain. In embodiments, the CAR-binding domain is a tumor necrosis factor (TNF) receptor. In embodiments, the CAR-binding domain is an interleukin cytokine. In embodiments, the CAR-binding domain is an interleukin receptor. In embodiments, the CAR-binding domain is a non-classical serine protease. In embodiments, the CAR-binding domain is a B cell surface molecule. In embodiments, the CAR-binding domain is a T-cell surface molecule. In embodiments, the CAR-binding domain is a natural killer (NK) cell surface molecule. In embodiments, the CAR-binding domain is a mucin. In embodiments, the CAR-binding domain is a cell adhesion molecule. In embodiments, the CAR-binding domain is a disialoganglioside. In embodiments, the CAR-binding domain is a C-type lectin-like receptor. In embodiments, the CAR-binding domain is a tight junction protein. In embodiments, the CAR-binding domain is a Lewis antigen. In embodiments, the CAR-binding domain is a zinc metalloenzyme.

In embodiments, the CAR binding domain is a ROR domain. In embodiments, the CAR-binding domain is a ROR1 domain. In embodiments, the CAR-binding domain is a ROR1 Ig-like domain. In embodiments, the CAR-binding domain includes the amino acid sequence of SEQ ID NO:1. In embodiments, the CAR-binding domain is the amino acid sequence of SEQ ID NO: 1.

In embodiments, the CAR binding domain is a ROR2 domain. In embodiments, the CAR-binding domain is a ROR2 Ig-like domain. In embodiments, the CAR-binding domain is a CD19 domain.

In embodiments, the CAR-binding domain is a TNFRSF17 domain, a TNFRSF8 domain, a CD5 domain, a ULBP1 domain, a ULBP2 domain, a CEACAM5 domain, a CD276 domain, an IL3RA domain, a MS4A1 domain, a CD33 domain, a NCAM1 domain, a MET domain, a CSPG4 domain, a SDC1 domain, a CD22 domain, a CD38 domain, a CD70 domain, an IL1RAP domain, an EGFR domain, a CD133 domain, an EGFRvIII domain, an EPCAM domain, an EPHA2 domain, an ERBB2 domain, a GPC3 domain, a MSLN domain, a TEM1 domain, a MUC1 domain, a PDCD1 domain, a CD274 domain, a KDR domain, an IL13RA2 domain, a FOLH1 domain, a GPNMB domain, a FAP domain, a CA9 domain, a FOLR1 domain, a L1CAM domain, a CD23 domain, a PSCA domain, a CD44 domain, a CD174 domain, a SLAMF7 domain, a GD2 domain, a BCMA domain, a CD30 domain, a CD4 domain, a CD7 domain, a CD79B domain, a CLEC12A domain, a Siglec-2 domain, a Siglec-3 domain, a SIRP alpha domain, a uPAR domain, a VEGFR2 domain, a GUCY2C domain, or a Protein L domain.

In embodiments, the CAR-binding domain is a TNFRSF17 domain. In embodiments, the CAR-binding domain is a TNFRSF8 domain. In embodiments, the CAR-binding domain is a CD5 domain. In embodiments, the CAR-binding domain is a ULBP1 domain. In embodiments, the CAR-binding domain is a ULBP2 domain. In embodiments, the CAR-binding domain is a CEACAM5 domain. In embodiments, the CAR-binding domain is a CD276 domain. In embodiments, the CAR-binding domain is an IL3RA domain. In embodiments, the CAR-binding domain is a MS4A1 domain. In embodiments, the CAR-binding domain is a CD33 domain. In embodiments, the CAR-binding domain is a NCAM1 domain. In embodiments, the CAR-binding domain is a MET domain. In embodiments, the CAR-binding domain is a CSPG4 domain. In embodiments, the CAR-binding domain is a SDC1 domain. In embodiments, the CAR-binding domain is a CD22 domain. In embodiments, the CAR-binding domain is a CD38 domain. In embodiments, the CAR-binding domain is a CD70 domain. In embodiments, the CAR-binding domain is an IL1RAP domain. In embodiments, the CAR-binding domain is an EGFR domain. In embodiments, the CAR-binding domain is a CD133 domain. In embodiments, the CAR-binding domain is an EGFRvIII domain. In embodiments, the CAR-binding domain is an EPCAM domain. In embodiments, the CAR-binding domain is an EPHA2 domain. In embodiments, the CAR-binding domain is an ERBB2 domain. In embodiments, the CAR-binding domain is a GPC3 domain. In embodiments, the CAR-binding domain is a MSLN domain. In embodiments, the CAR-binding domain is a TEM1 domain. In embodiments, the CAR-binding domain is a MUC1 domain. In embodiments, the CAR-binding domain is a PDCD1 domain. In embodiments, the CAR-binding domain is a CD274 domain. In embodiments, the CAR-binding domain is a KDR domain. In embodiments, the CAR-binding domain is an IL13RA2 domain. In embodiments, the CAR-binding domain is a FOLH1 domain. In embodiments, the CAR-binding domain is a GPNMB domain. In embodiments, the CAR-binding domain is a FAP domain. In embodiments, the CAR-binding domain is a CA9 domain. In embodiments, the CAR-binding domain is a FOLR1 domain. In embodiments, the CAR-binding domain is a L1CAM domain, a CD23 domain. In embodiments, the CAR-binding domain is a PSCA domain. In embodiments, the CAR-binding domain is a CD44 domain. In embodiments, the CAR-binding domain is a CD174 domain. In embodiments, the CAR-binding domain is a SLAMF7 domain. In embodiments, the CAR-binding domain is a GD2 domain. In embodiments, the CAR-binding domain is a BCMA domain. In embodiments, the CAR-binding domain is a CD30 domain. In embodiments, the CAR-binding domain is a CD4 domain. In embodiments, the CAR-binding domain is a CD7 Domain. In embodiments, the CAR-binding domain is a CD79B domain. In embodiments, the CAR-binding domain is a CLEC12A domain. In embodiments, the CAR-binding domain is a Siglec-2 domain. In embodiments, the CAR-binding domain is a Siglec-3 domain. In embodiments, the CAR-binding domain is a SIRP alpha domain. In embodiments, the CAR-binding domain is a uPAR domain, a VEGFR2 domain. In embodiments, the CAR-binding domain is a GUCY2C domain. In embodiments, the CAR-binding domain is a Protein L domain.

In embodiments, the ligand binding domain includes an amino acid sequence of about 1 to about 500 amino acids in length. In embodiments, the ligand binding domain includes an amino acid sequence of about 2 to about 500 amino acids in length. In embodiments, the ligand binding domain includes an amino acid sequence of about 3 to about 500 amino acids in length. In embodiments, the ligand binding domain includes an amino acid sequence of about 4 to about 500 amino acids in length. In embodiments, the ligand binding domain includes an amino acid sequence of about 5 to about 500 amino acids in length. In embodiments, the ligand binding domain includes an amino acid sequence of about 6 to about 500 amino acids in length. In embodiments, the ligand binding domain includes an amino acid sequence of about 7 to about 500 amino acids in length. In embodiments, the ligand binding domain includes an amino acid sequence of about 8 to about 500 amino acids in length. In embodiments, the ligand binding domain includes an amino acid sequence of about 9 to about 500 amino acids in length. In embodiments, the ligand binding domain includes an amino acid sequence of about 10 to about 500 amino acids in length. In embodiments, the ligand binding domain includes an amino acid sequence of about 15 to about 500 amino acids in length. In embodiments, the ligand binding domain includes an amino acid sequence of about 20 to about 500 amino acids in length. In embodiments, the ligand binding domain includes an amino acid sequence of about 25 to about 500 amino acids in length. In embodiments, the ligand binding domain includes an amino acid sequence of about 50 to about 500 amino acids in length. In embodiments, the ligand binding domain includes an amino acid sequence of about 75 to about 500 amino acids in length. In embodiments, the ligand binding domain includes an amino acid sequence of about 100 to about 500 amino acids in length. In embodiments, the ligand binding domain includes an amino acid sequence of about 150 to about 500 amino acids in length. In embodiments, the ligand binding domain includes an amino acid sequence of about 200 to about 500 amino acids in length. In embodiments, the ligand binding domain includes an amino acid sequence of about 250 to about 500 amino acids in length. In embodiments, the ligand binding domain includes an amino acid sequence of about 300 to about 500 amino acids in length. In embodiments, the ligand binding domain includes an amino acid sequence of about 350 to about 500 amino acids in length. In embodiments, the ligand binding domain includes an amino acid sequence of about 400 to about 500 amino acids in length. In embodiments, the ligand binding domain includes an amino acid sequence of about 450 to about 500 amino acids in length.

In embodiments, the ligand binding domain includes an amino acid sequence of about 1 to about 450 amino acids in length. In embodiments, the ligand binding domain includes an amino acid sequence of about 1 to about 400 amino acids in length. In embodiments, the ligand binding domain includes an amino acid sequence of about 1 to about 350 amino acids in length. In embodiments, the ligand binding domain includes an amino acid sequence of about 1 to about 300 amino acids in length. In embodiments, the ligand binding domain includes an amino acid sequence of about 1 to about 250 amino acids in length. In embodiments, the ligand binding domain includes an amino acid sequence of about 1 to about 200 amino acids in length. In embodiments, the ligand binding domain includes an amino acid sequence of about 1 to about 150 amino acids in length. In embodiments, the ligand binding domain includes an amino acid sequence of about 1 to about 100 amino acids in length. In embodiments, the ligand binding domain includes an amino acid sequence of about 1 to about 75 amino acids in length. In embodiments, the ligand binding domain includes an amino acid sequence of about 1 to about 50 amino acids in length. In embodiments, the ligand binding domain includes an amino acid sequence of about 1 to about 25 amino acids in length. In embodiments, the ligand binding domain includes an amino acid sequence of about 1 to about 20 amino acids in length. In embodiments, the ligand binding domain includes an amino acid sequence of about 1 to about 15 amino acids in length. In embodiments, the ligand binding domain includes an amino acid sequence of about 1 to about 10 amino acids in length. In embodiments, the ligand binding domain includes an amino acid sequence of about 1 to about 9 amino acids in length. In embodiments, the ligand binding domain includes an amino acid sequence of about 1 to about 8 amino acids in length. In embodiments, the ligand binding domain includes an amino acid sequence of about 1 to about 7 amino acids in length. In embodiments, the ligand binding domain includes an amino acid sequence of about 1 to about 6 amino acids in length. In embodiments, the ligand binding domain includes an amino acid sequence of about 1 to about 5 amino acids in length. In embodiments, the ligand binding domain includes an amino acid sequence of about 1 to about 4 amino acids in length. In embodiments, the ligand binding domain includes an amino acid sequence of about 1 to about 3 amino acids in length. In embodiments, the ligand binding domain includes an amino acid sequence of about 1 to about 2 amino acids in length.

In embodiments, the ligand binding domain includes an amino acid sequence of 1 to 500 amino acids in length. In embodiments, the ligand binding domain includes an amino acid sequence of 2 to 500 amino acids in length. In embodiments, the ligand binding domain includes an amino acid sequence of 3 to 500 amino acids in length. In embodiments, the ligand binding domain includes an amino acid sequence of 4 to 500 amino acids in length. In embodiments, the ligand binding domain includes an amino acid sequence of 5 to 500 amino acids in length. In embodiments, the ligand binding domain includes an amino acid sequence of 6 to 500 amino acids in length. In embodiments, the ligand binding domain includes an amino acid sequence of 7 to 500 amino acids in length. In embodiments, the ligand binding domain includes an amino acid sequence of 8 to 500 amino acids in length. In embodiments, the ligand binding domain includes an amino acid sequence of 9 to 500 amino acids in length. In embodiments, the ligand binding domain includes an amino acid sequence of 10 to 500 amino acids in length. In embodiments, the ligand binding domain includes an amino acid sequence of 15 to 500 amino acids in length. In embodiments, the ligand binding domain includes an amino acid sequence of 20 to 500 amino acids in length. In embodiments, the ligand binding domain includes an amino acid sequence of 25 to 500 amino acids in length. In embodiments, the ligand binding domain includes an amino acid sequence of 50 to 500 amino acids in length. In embodiments, the ligand binding domain includes an amino acid sequence of 75 to 500 amino acids in length. In embodiments, the ligand binding domain includes an amino acid sequence of 100 to 500 amino acids in length. In embodiments, the ligand binding domain includes an amino acid sequence of 150 to 500 amino acids in length. In embodiments, the ligand binding domain includes an amino acid sequence of 200 to 500 amino acids in length. In embodiments, the ligand binding domain includes an amino acid sequence of 250 to 500 amino acids in length. In embodiments, the ligand binding domain includes an amino acid sequence of 300 to 500 amino acids in length. In embodiments, the ligand binding domain includes an amino acid sequence of 350 to 500 amino acids in length. In embodiments, the ligand binding domain includes an amino acid sequence of 400 to 500 amino acids in length. In embodiments, the ligand binding domain includes an amino acid sequence of 450 to 500 amino acids in length.

In embodiments, the ligand binding domain includes an amino acid sequence of 1 to 450 amino acids in length. In embodiments, the ligand binding domain includes an amino acid sequence of 1 to 400 amino acids in length. In embodiments, the ligand binding domain includes an amino acid sequence of 1 to 350 amino acids in length. In embodiments, the ligand binding domain includes an amino acid sequence of 1 to 300 amino acids in length. In embodiments, the ligand binding domain includes an amino acid sequence of 1 to 250 amino acids in length. In embodiments, the ligand binding domain includes an amino acid sequence of 1 to 200 amino acids in length. In embodiments, the ligand binding domain includes an amino acid sequence of 1 to 150 amino acids in length. In embodiments, the ligand binding domain includes an amino acid sequence of 1 to 100 amino acids in length. In embodiments, the ligand binding domain includes an amino acid sequence of 1 to 75 amino acids in length. In embodiments, the ligand binding domain includes an amino acid sequence of 1 to 50 amino acids in length. In embodiments, the ligand binding domain includes an amino acid sequence of 1 to 25 amino acids in length. In embodiments, the ligand binding domain includes an amino acid sequence of 1 to 20 amino acids in length. In embodiments, the ligand binding domain includes an amino acid sequence of 1 to 15 amino acids in length. In embodiments, the ligand binding domain includes an amino acid sequence of 1 to 10 amino acids in length. In embodiments, the ligand binding domain includes an amino acid sequence of 1 to 9 amino acids in length. In embodiments, the ligand binding domain includes an amino acid sequence of 1 to 8 amino acids in length. In embodiments, the ligand binding domain includes an amino acid sequence of 1 to 7 amino acids in length. In embodiments, the ligand binding domain includes an amino acid sequence of 1 to 6 amino acids in length. In embodiments, the ligand binding domain includes an amino acid sequence of 1 to 5 amino acids in length. In embodiments, the ligand binding domain includes an amino acid sequence of 1 to 4 amino acids in length. In embodiments, the ligand binding domain includes an amino acid sequence of 1 to 3 amino acids in length. In embodiments, the ligand binding domain includes an amino acid sequence of 1 to 2 amino acids in length.

In embodiments, the ligand binding domain is an antibody domain. In embodiments, the ligand binding domain is an Fc domain. In embodiments, the ligand binding domain includes the amino acid sequence of SEQ ID NO:3. In embodiments, the ligand binding domain is the amino acid sequence of SEQ ID NO:3.

In embodiments, the detectable moiety is a fluorescent moiety. In embodiments, the detectable moiety is a phycoerythrin (PE) moiety.

In embodiments, the chimeric binding peptide comprises a ROR1 domain, an Fc domain and one or more PE moieties. In embodiments, the chimeric binding peptide includes the amino acid sequence of SEQ ID NO:4. In embodiments, the chimeric binding peptide is the amino acid sequence of SEQ ID NO:4. In embodiments, the complex includes a ROR1 CAR T-cell, a ROR1 domain, an Fc domain and one or more PE moieties.

### Pharmaceutical Compositions

Provided herein, inter alia, are pharmaceutical compositions including a purified CAR T-cell population which has high CAR expression. Thus, in an aspect is provided a pharmaceutical composition including a therapeutically effective amount of the purified CAR T-cell population provided herein including embodiments thereof and a pharmaceutically acceptable excipient.

### Methods of Treatment

Provided herein, inter alia, are methods of treating cancer in a subject in need thereof. The purified CAR T-cell population generated using the methods described herein included embodiments thereof are efficient at targeting and killing cancer cells in vitro and in vivo. Thus, in an aspect is provided a method of treating cancer in a subject in need thereof including administering to the subject a therapeutically effective amount of the purified CAR T-cell provided herein including embodiments thereof or the pharmaceutical composition provided herein including embodiments thereof, thereby treating the cancer.

In embodiments, the cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer. In embodiments, the cancer is lymphoma. In embodiments, the cancer is chronic lymphocytic leukemia (CLL). In embodiments, the cancer is small lymphocytic lymphoma. In embodiments, the cancer is marginal cell B-Cell lymphoma. In embodiments, the cancer is Burkett's Lymphoma. In embodiments, the cancer is leukemia. In embodiments, the cancer is chronic B cell leukemia. In embodiments, the cancer is renal cell carcinoma. In embodiments, the cancer is colon cancer. In embodiments, the cancer is colorectal cancer. In embodiments, the cancer is breast cancer. In embodiments, the cancer is epithelial squamous cell cancer. In embodiments, the cancer is melanoma. In embodiments, the cancer is myeloma. In embodiments, the cancer is stomach cancer. In embodiments, the cancer is brain cancer. In embodiments, the cancer is lung cancer, pancreatic cancer. In embodiments, the cancer is cervical cancer. In embodiments, the cancer is ovarian cancer. In embodiments, the cancer is liver cancer. In embodiments, the cancer is bladder cancer. In embodiments, the cancer is prostate cancer. In embodiments, the cancer is testicular cancer. In embodiments, the cancer is thyroid cancer. In embodiments, the cancer is head and neck cancer.

### EXAMPLES

### Example 1

The trafficking of human T-cells towards specific tumor antigens may be achieved by modifying human T-cells with transactivating chimeric antigen receptors (CARS). The transactivated CAR molecules have been in development since the 1980s and consist of an extracellular antigen binding moiety (single chain variable fragment (scFv) derived most often from a monoclonal antibody (mAb)) bound to an intracellular signaling domain through a flexible stalk. CAR T-cells have been generated that employed scFv generated from mAbs that target ROR1 and ROR2. For the generation of CAR T-cells, Peripheral Blood Mononuclear Cell (PBMC) from donor human leukapheresed samples were isolated. The collected PBMC were further processed to isolate CD3^{pos} T-cells that express the lineage defining proteins CD4 and CD8. The isolated bulk lymphocyte population was then activated with CD3/CD28 expressing microbeads and then transduced with lentivirus expression construct to generate the anti-tumor CAR-T product.

To generate a purified more homogenous product that we can control the relative activity of the product, we devised a purification method to isolate our anti-ROR1 and ROR2 CAR products. The invention that we have developed can also be easily employed to isolate T-cell CARs generated from other mAbs and scFv and be used to generate purified T-cell CARs that will have enhanced specific activities. For this process, first isolate PBMC from donor human leukapheresed samples. The collected PBMC are further processed to isolate CD3^{pos} T-cells that express the lineage defining proteins CD4 and CD8. This bulk lymphocyte population is then activated with CD3/CD28 expressing microbeads and then transduced with our lentivirus expression construct to generate the anti-tumor CAR-T product.

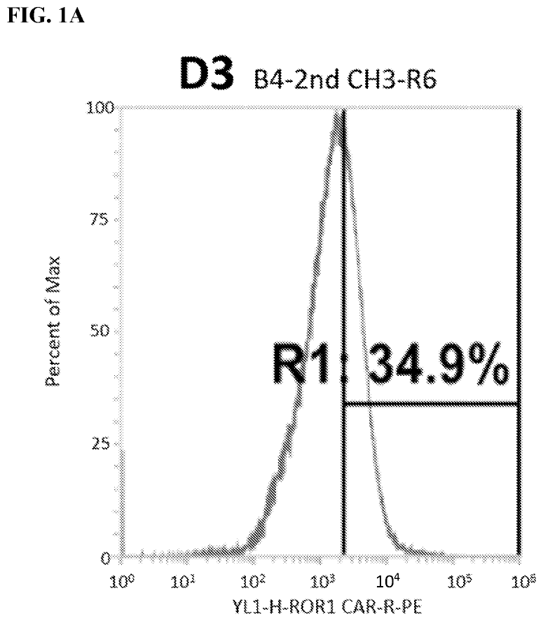

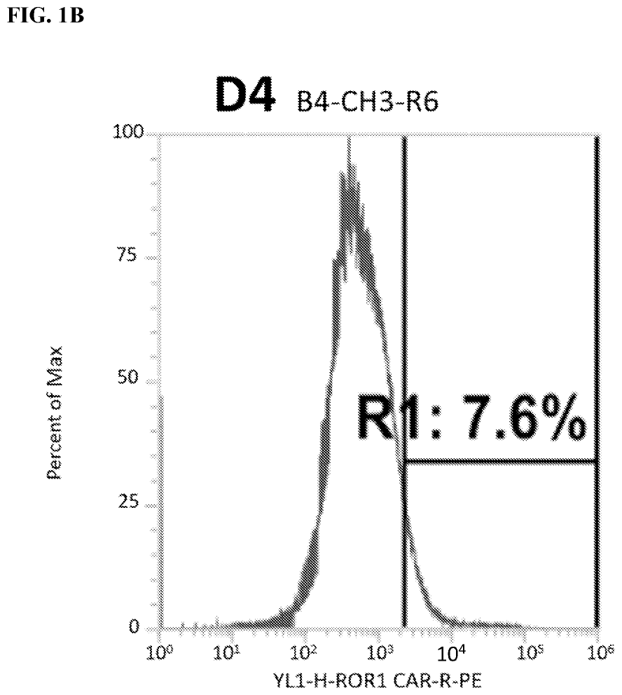

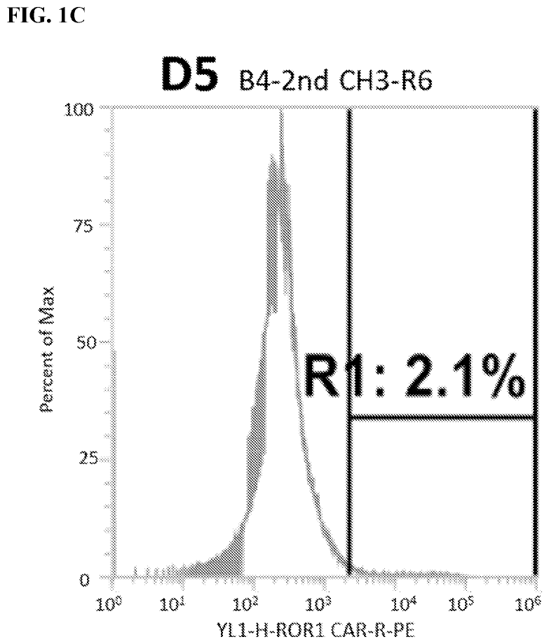

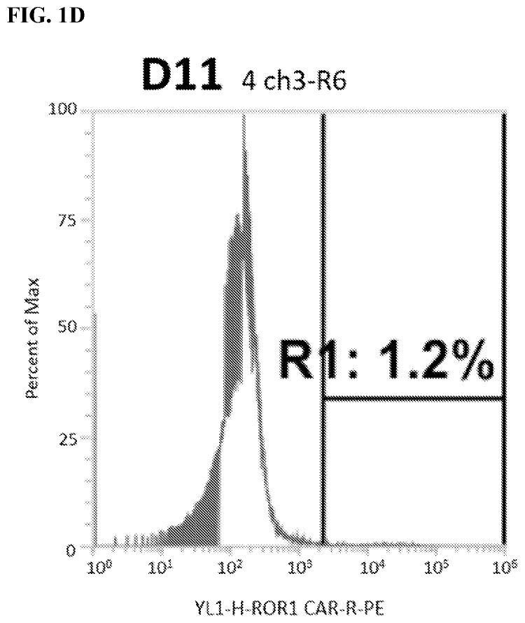

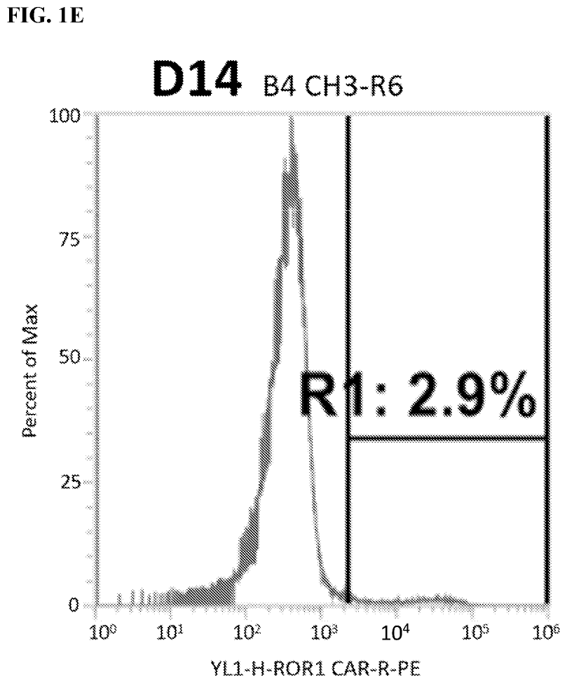

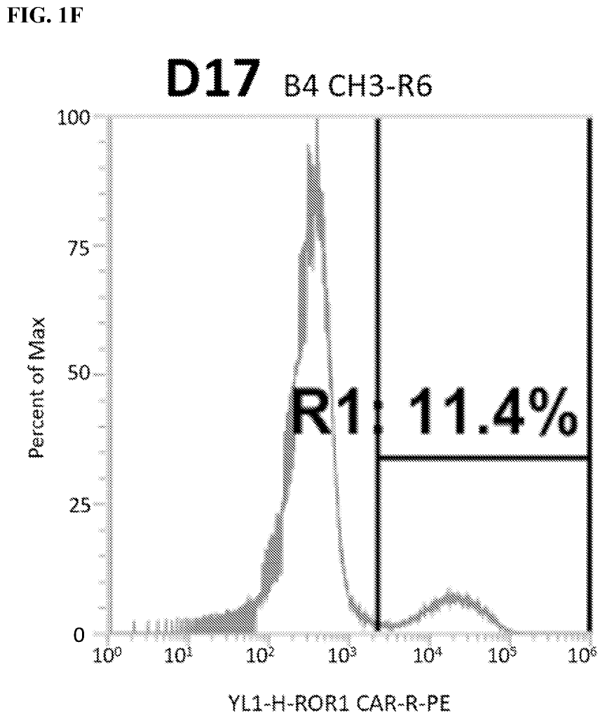

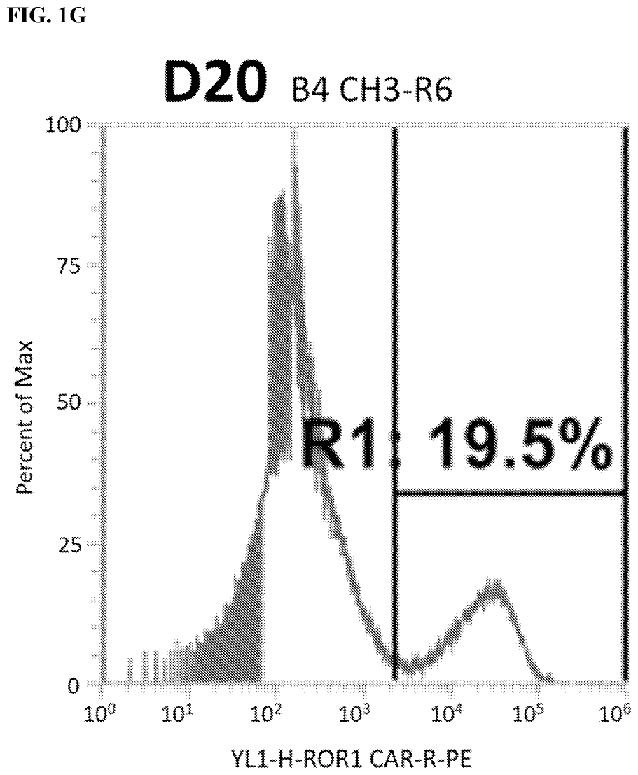

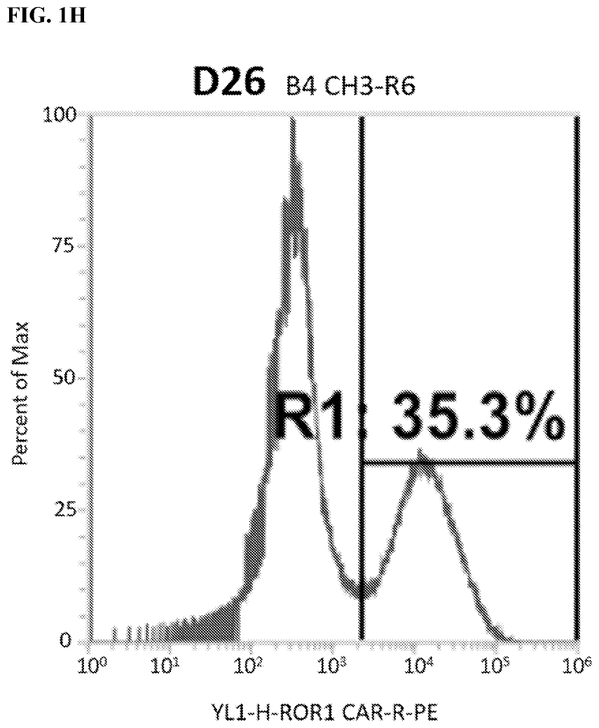

As shown in FIGS. 1A-1I, the generation of the T-cell CAR takes at least 14 days before any specific activity of the CAR product was detected. Several studies were conducted and the activity of generated CAR products in in-vitro and in-vivo studies were dependent on the 2nd high ROR-Ig binding peak shown in FIGS. 1A-1I Since the emergence of CAR product from the bulk T-cell population, which was dependent on the expansion of population, to exceed the cells in the bulk culture (signal to noise). Dozens of CAR T-cell products were generated from individual donor human T-cells and the eventual relative expression of the active peak was variable. Therefore, to generate a purified more homogenous product that may be controlled by the relative activity of the product, a purification method was devised to isolate anti-Tyrosine-protein kinase transmembrane receptors known as anti-ROR1 and ROR2 CAR products. The current subject matter that was developed may also be easily employed to isolate T-cell CARs generated from other mAbs and scFv and may be used to generate purified T-cell CARs that will have enhanced specific activities.

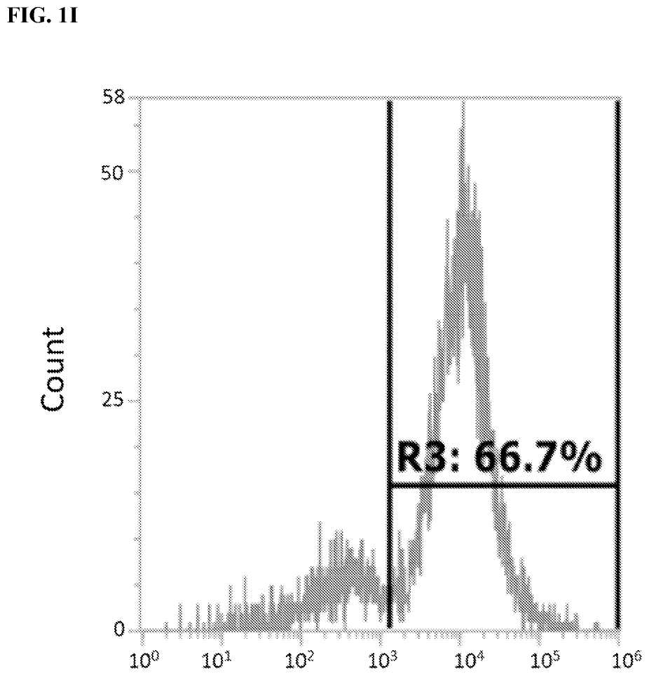

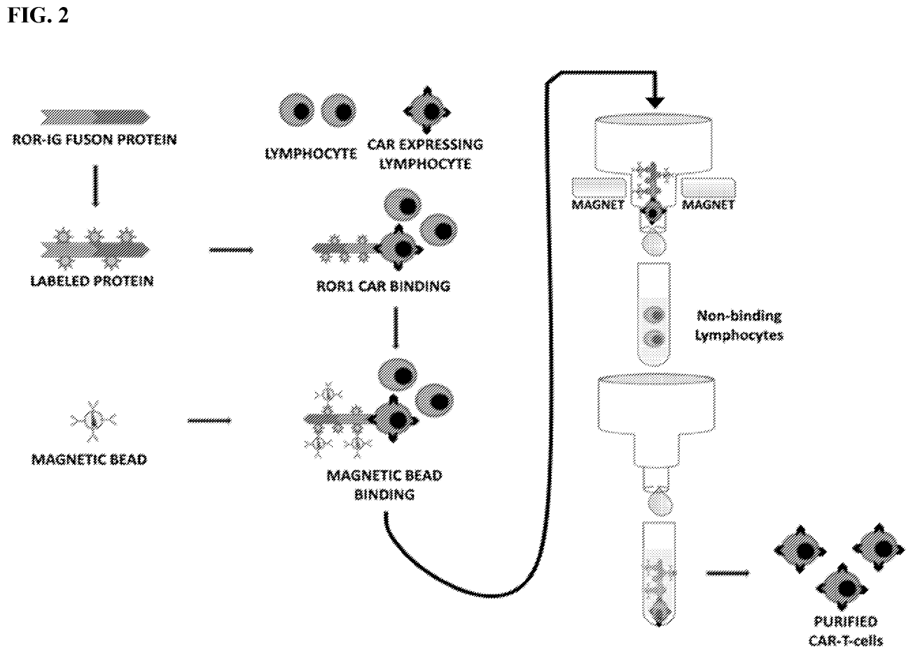

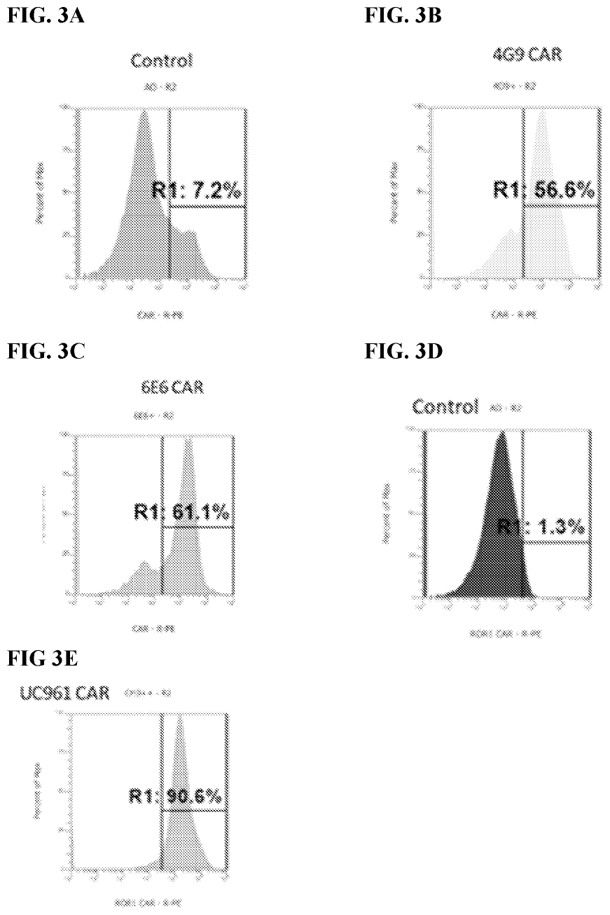

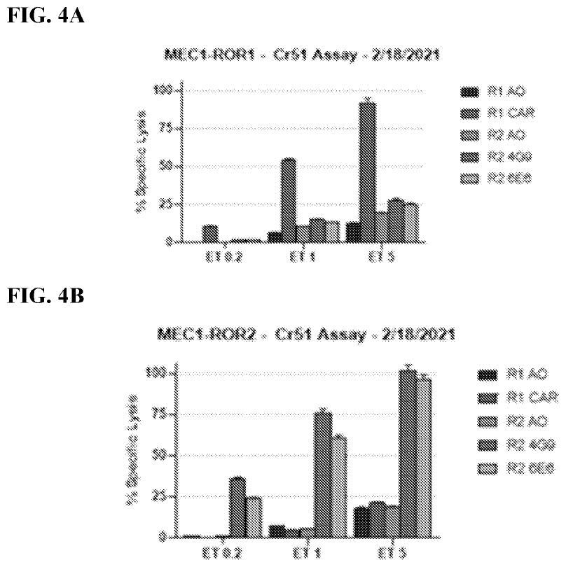

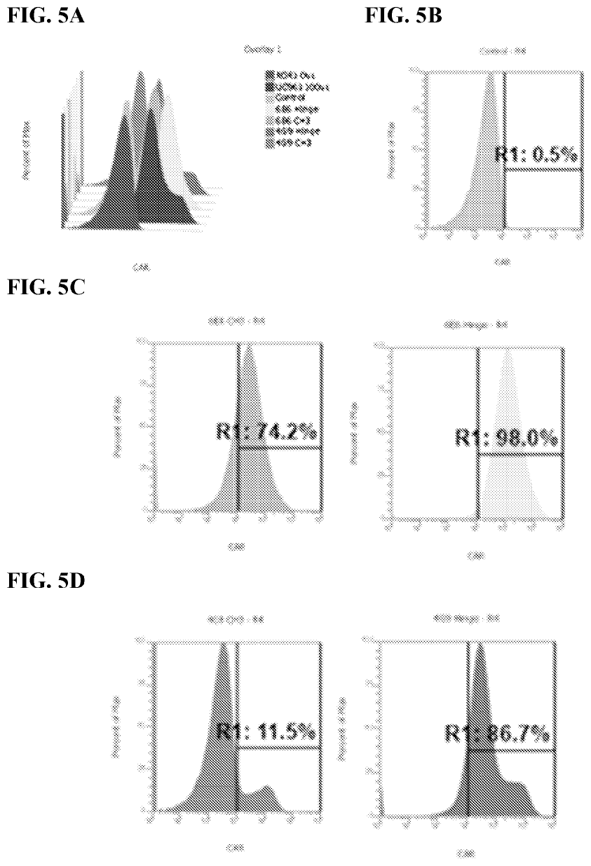

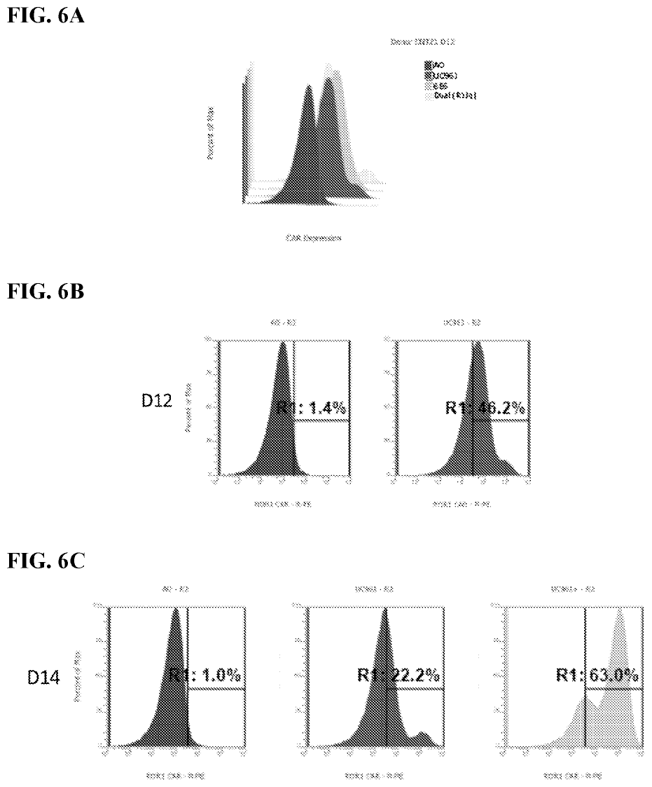

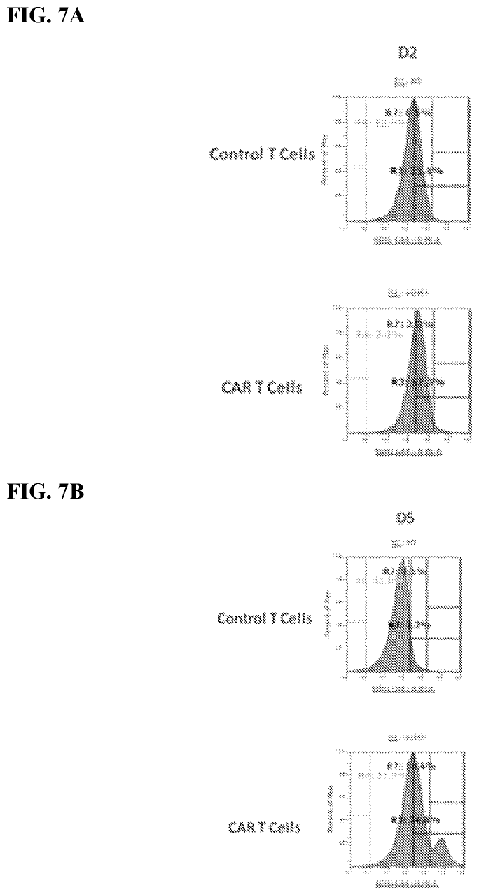

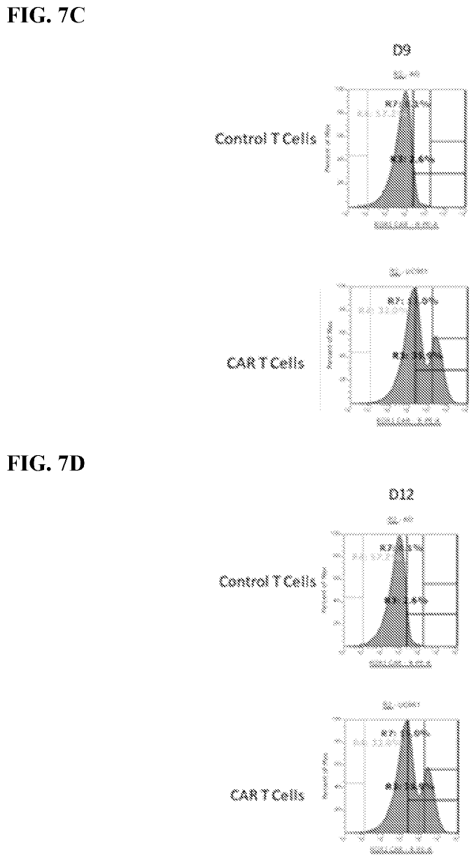

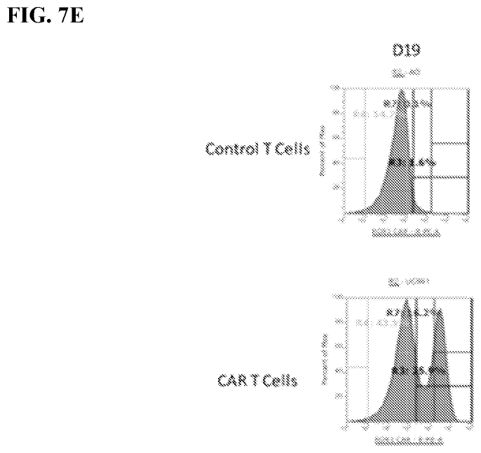

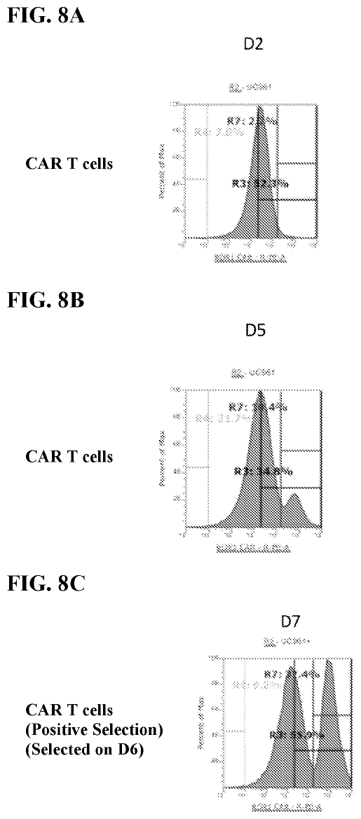

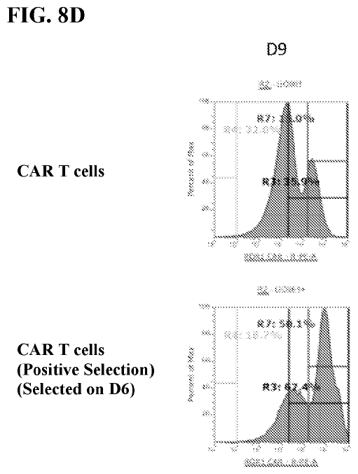

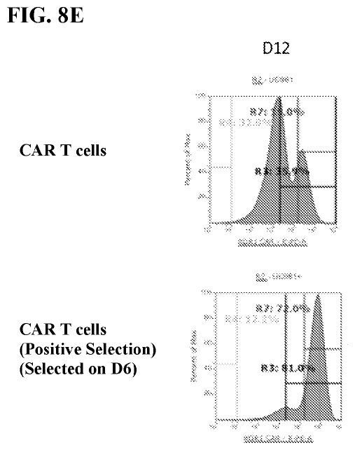

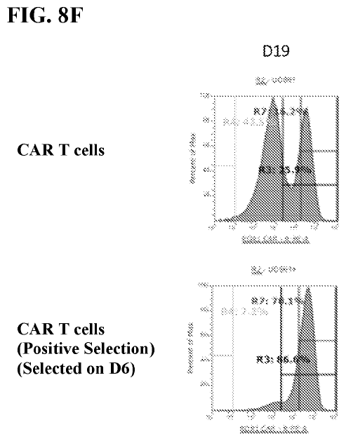

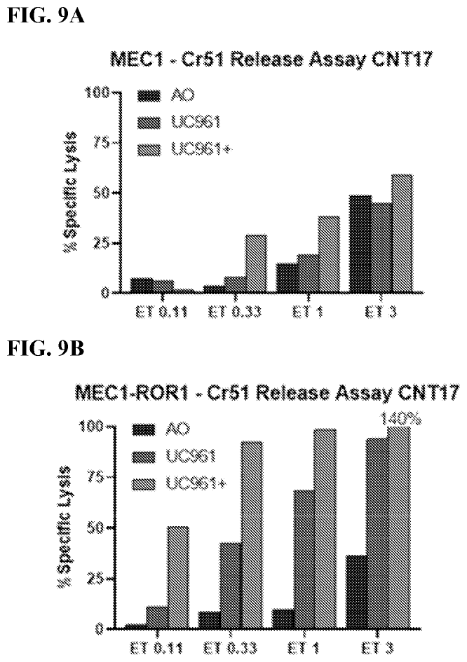

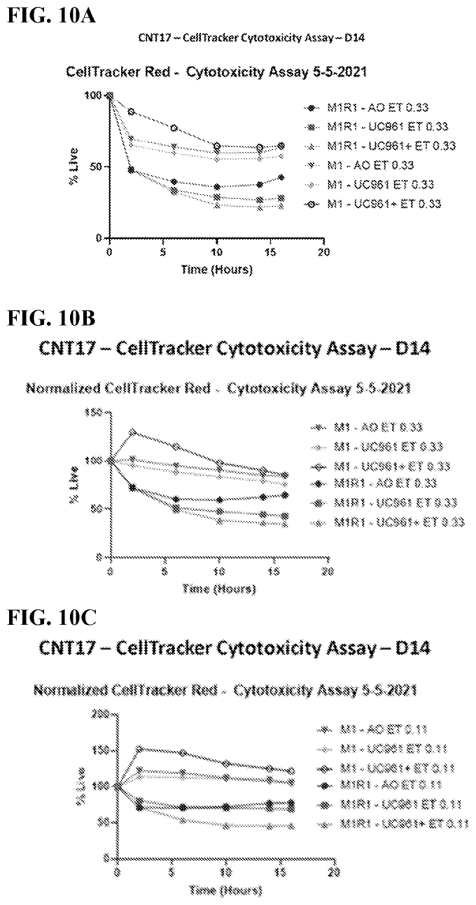

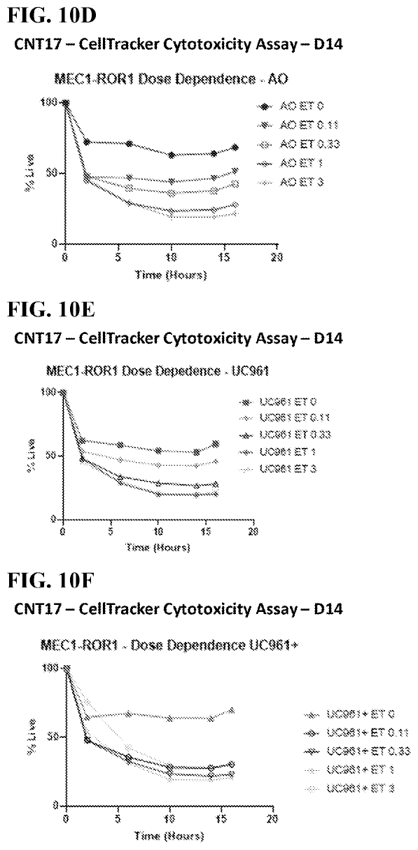

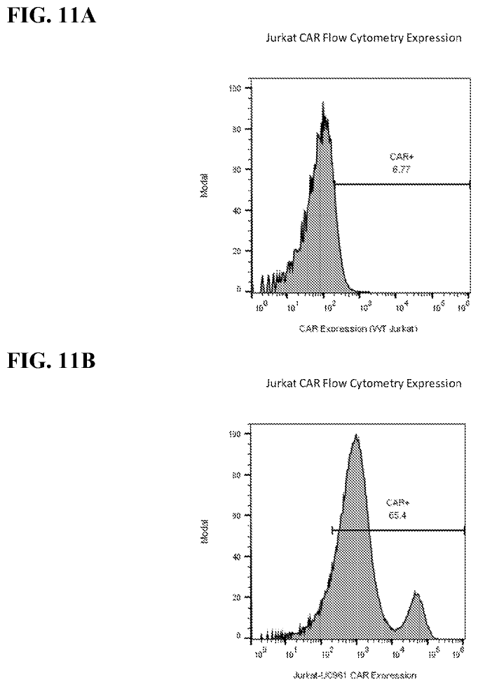

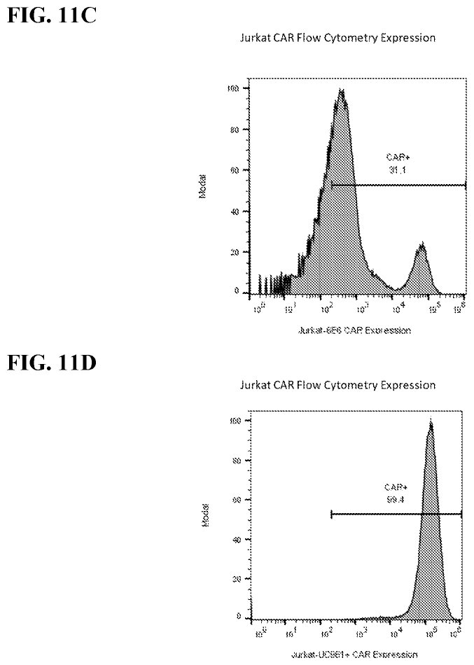

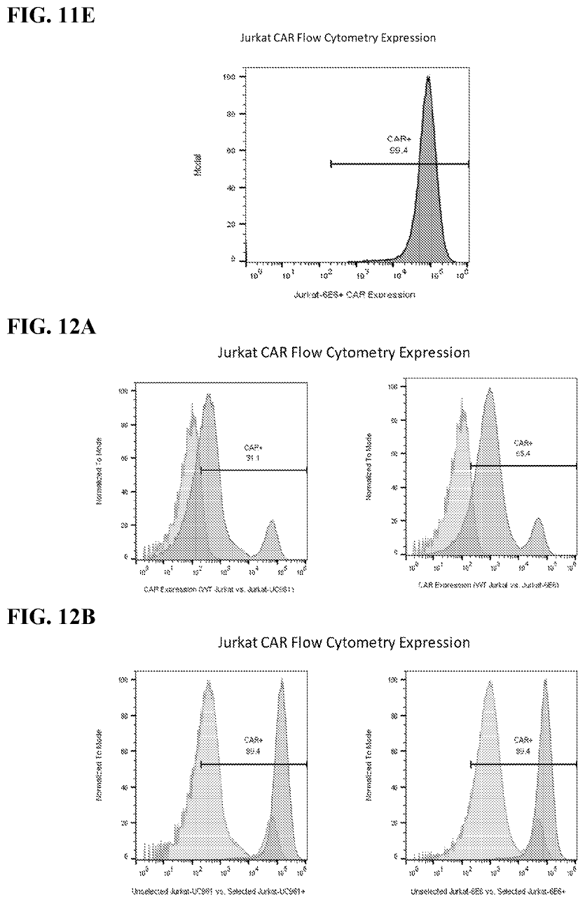

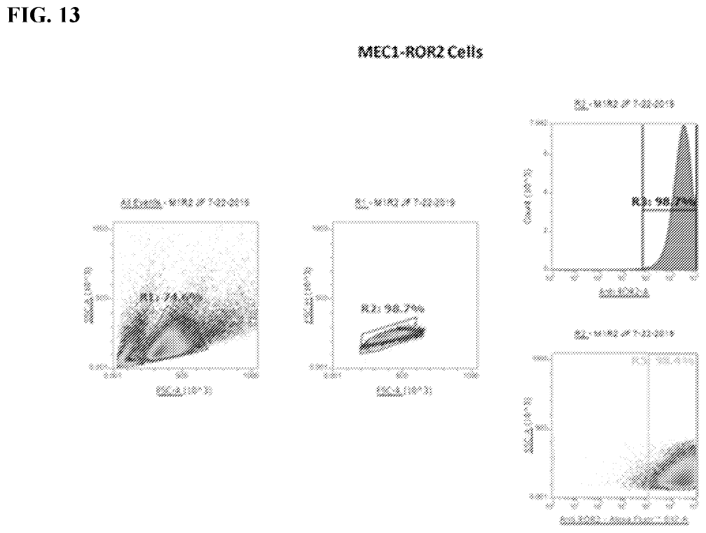

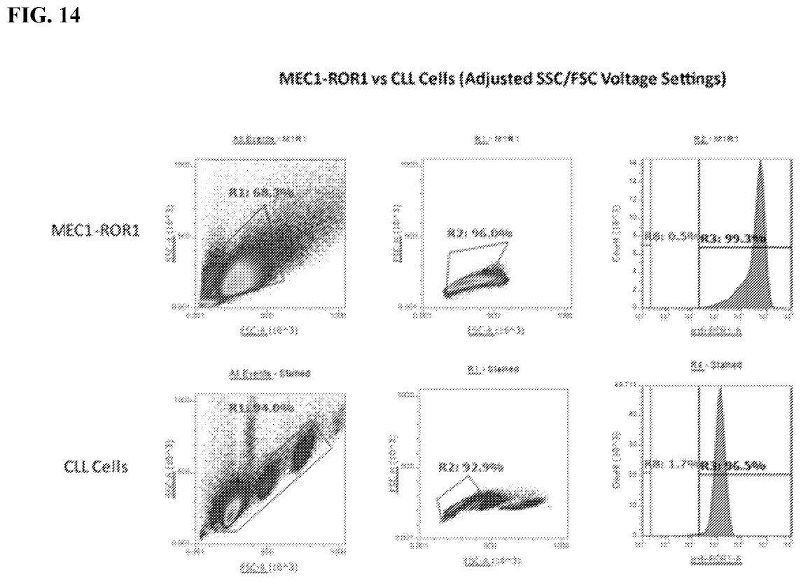

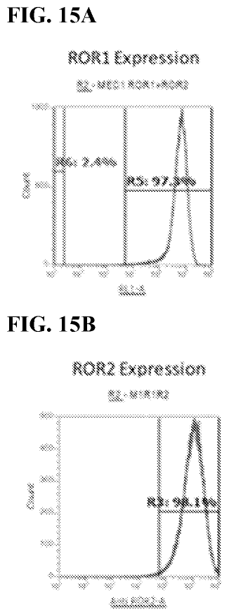

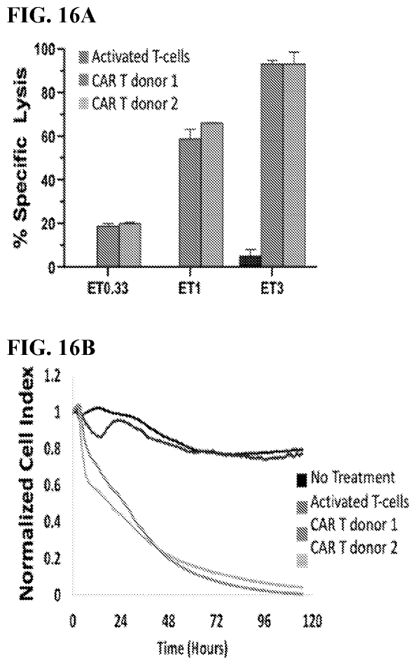

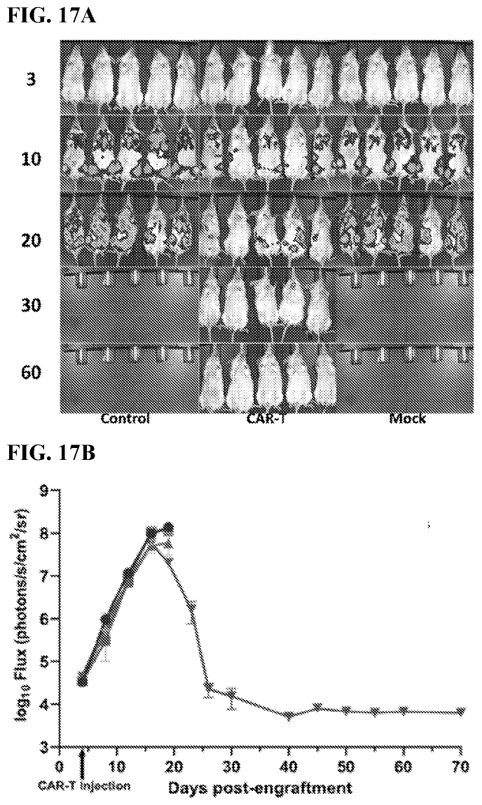

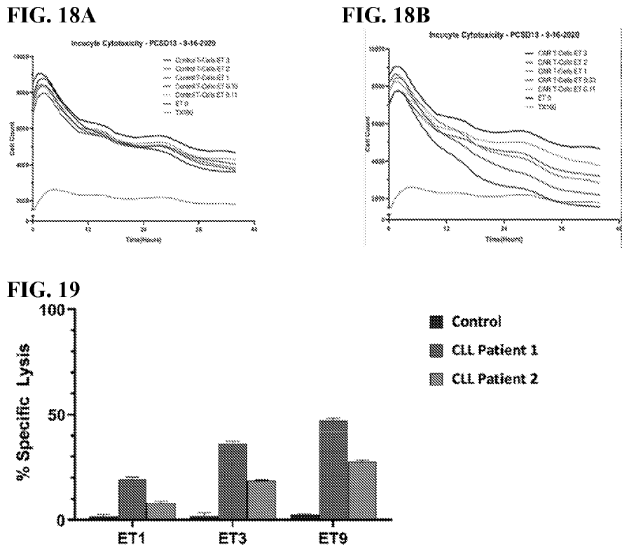

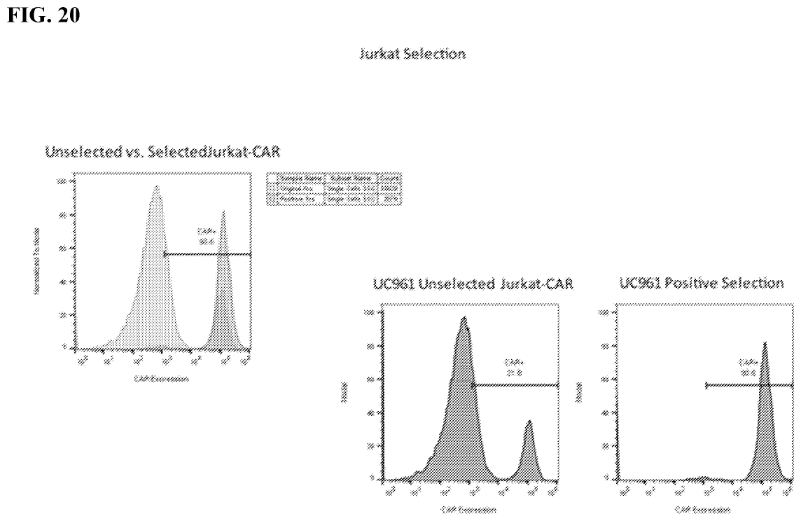

In the first step, a fusion product that expressed the target antigen (for example ROR1-Ig) was labeled with an antigenic label (a fluorescent stain for example Alex 647 or PE) and isolation of the labeled cells was then accomplished using magnetic microbeads. For isolation of the labeled cells, Miltenyi has magnetic cell isolation beads that may target the fluorescent markers Alexa 647, APC, FITC and PE, which were part of their multi-sort Kits that employ fluorescent mAbs to isolate the target cells. Following the positive selection, the magnetic particles were removed from the cells by using commercially available releasing reagents (FIG. 2).

As shown in FIGS. 1A-1I, isolation of active T-cell CARs from the bulk transduced human T-cells were regularly used to purify generated anti-ROR1 CAR product and were involved in the process of transitioning anti-ROR1 CAR into a GMP compatible process for clinical application.

To use the commercial product as described, one would need an antibody to bind to the target. For a CAR that is targeted to a specific antigen, one would need an antibody that binds to the scFv, which would be an anti-idiotype antibody. Making an antibody to work against the target can be difficult to do and may impact the activity of the targeted T-cell CAR product because of the strong avidity of the immune molecule for the target. For the cirmtuzumab-based ROR1 targeting scFv this anti-idiotype antibody does not exist and it would be difficult to make one that would work with for present invention.

Making a protein fusion product is very simple and readily adaptable to any targeted scFv (for instance a CD19-Ig Fusion protein exists). Therefore, the invention could use the many available commercial fusion proteins to purify the CARs including those that have been approved for clinical use.

The current invention would be beneficial for anyone conducting preclinical and clinical studies with CAR T-cells. It would of great interest to major pharmaceutical companies that have approved CAR products, and other companies that are advancing T-cell CARS into clinical studies.

### Example 2: T-Cell Culture and Transduction Protocol

Human primary T-cells were obtained by directly isolating them from either human donor buffy coat or leukapheresis (apheresis) product. The healthy donor specimens used in this study were acquired from the San Diego Blood Bank. For B-cell CLL patient specimens that met the required diagnostics and immunophenotypic criteria, they were collected from the University of California, San Diego (UCSD) Moores Cancer Center with written and informed consent from the patients. This study was conducted in compliance with the Declaration of Helsinki and the UC San Diego institutional review board guidelines.

T-cells were isolated directly from donor and patient blood products using StraightFrom MicroBeads (Miltenyi Biotec) per standard protocol. Automated separation was carried out to isolate CD4, CD8, and/or CD3 T-cell sub-populations using the autoMACS Pro Separator (Miltenyi Biotec). T-cells were cultured in ImmunoCult™-XF T-cell Expansion Medium (Stemcell Technologies #10981) supplemented with Pen/Strep (Gibco), gentamycin (VWR), and 25 ng/ml of IL-7 and IL-15 (Miltenyi Biotec) in G-Rex6 or gRex100 (Wilson Wolf). T-cell TransAct (Miltenyi Biotec) was added per standard protocol for T-cell stimulation. 1-3 days after activation, lentiviruses carrying the constructs of interest were added at MOI 10 with 10 μg/mL protamine sulfate (Sigma-Aldrich) or 100 ug/mL Synperonic F108 (Sigma-Aldrich) as a transducing agent. Media was exchanged with the standard culture media the following day and replenished every 2-4 days during expansion.

Additional transducing reagents that are contemplated, include but are not limited to: (1) Synperonic F108: Non-ionic surfactant used to increase the efficiency of lentiviral transduction. Recommended concentration is 0.01-0.05%; (2) Lentiboost: Transduction enhancer used to improve viral transduction efficiency. Recommended concentration is 8 μg/mL; (3) Protamine sulfate: Cationic peptide used to neutralize the negative charge on the cell surface and enhance viral attachment. Recommended concentration is 2-8 μg/mL; and (4) Polybrene: Cationic polymer that can increase viral attachment to cells and enhance transduction efficiency. Recommended concentration is 4-8 μg/mL.

### Example 3

The trafficking of human T-cells towards specific tumor antigens can be achieved by modifying these cells with transactivating chimeric antigen receptors (CARS). These CAR molecules have been in development since the 1980s and consist of an extracellular antigen binding moiety (single chain variable fragment (scFv) derived most often from a monoclonal antibody (mAb)) bound to an intracellular signaling domain through a flexible stalk. We have generated CAR T-cells that employ scFv generated from mAbs that target ROR1 and ROR2. For this process, we first isolate PBMC from donor human leukapheresed samples. The collected PBMC are further processed to isolate CD3⁺ T-cells that express the lineage defining proteins CD4 and CD8. This bulk lymphocyte population is then activated with CD3/CD28 expressing microbeads and then transduced with our lentivirus expression construct to generate the anti-tumor CAR-T product. As shown in FIGS. 1A-1I, the generation of the T-cell CAR takes at least 14 days before any specific activity of the CAR product is detected. We have conducted many studies and the activity of our CAR products in in vitro and in vivo studies is dependent on the 2nd high ROR-Ig binding peak shown in FIGS. 1F-1H. Since the emergence of this product from the bulk T-cell population is dependent on the expansion of this population to exceed the cells in the bulk culture (signal to noise). We have generated dozens of CAR-T products from individual donor human T-cells and the eventual relative expression of the active peak is variable. Therefore, to generate a purified more homogenous product that we can control the relative activity of the product, we devised a purification method to isolate our anti-ROR1 and ROR2 CAR products. The invention that we have developed can also be easily employed to isolate T-cell CARs generated from other mAbs and scFv and be used to generate purified T-cell CARs that will have enhanced specific activities.

In the first step, a fusion product that expresses the target antigen (for example ROR1-Ig) is labeled with an antigenic label (a fluorescent stain for example Alex 647 or PE) and isolation of the labeled cells is then accomplished \ using magnetic microbeads. For this process Miltenyi has magnetic cell isolation beads that can target the fluorescent markers Alexa 647, APC, FITC and PE, which are part of their multi-sort Kits that employ fluorescent mAbs to isolate the target cells. Following the positive selection (i.e. purification), the magnetic particles are removed from the cells by using commercially available releasing reagents (FIG. 2).

### Example 4: Methods to Generate Purified CAR T-Cells

One method described herein is dependent on the use of a bifunctional “sandwich” protein. Bifunctional sandwich proteins are chimeric molecules that contain multiple functional sites that have been created as bridge or adaptor elements primarily for subsequent analysis. Bifunctional sandwich proteins have been used for multiple scientific applications including the quantification of biological products by ELISA, PCR or FLOW cytometry. One purification procedure described herein is based on the utilization of chimeric bifunctional proteins that: 1) bind to the CAR antigen binding domain, and 2) have secondary antigenic site(s) that can be bound by a secondary capture moiety including a site-directed secondary antibody or ligand.

In certain embodiments, the secondary binding domain can be recognized by an antibody or ligand attached to a solid matrix. In this manner the chimeric protein would be bound by the CAR-binding domain forming a bridge to the secondary binding site. In one embodiment, the secondary binding site can be bound by an antibody or ligand attached to a solid matrix to directly capture the CAR T-cell, while contaminating non-CAR expressing cells remain unbound. The solid matrix bound CAR T-cells can then be removed from the unbound cells by physical means including centrifugation or column chromatography. The purified CAR-T-cells can subsequently be detached from the solid matrix by enzymatic, physical or chemical means. In another method the secondary antibody can be attached to an iron containing solid matrix bead, which can then be used to magnetically remove the bound CAR T-cell from contaminating non-CAR expressing T-cells. The CAR-T-cells isolated in this manner can subsequently be detached from the solid matrix by enzymatic, physical or chemical means.

There are many commercial systems that can be used to affect the purification of the CAR-T-cells as described herein. In certain applications, this may include the use of iron coated beads conjugated to an antibody or ligand that binds to a common laboratory reagent such as R-Phycoerythrin (PE) or Alexaflor®647.

Procedure. The first step in this procedure is the generation of CAR modified cells using one of many established procedures for generating these cellular immunotherapies. During the generation of the CAR products, both modified cellular products that express a chimeric antigen receptor and non-modified cells will be contained in the final product. The relative expansion of these two cell populations will define the quality of the final product used therapeutically. Therefore, a highly purified product will maximize performance, decrease off-target activities and toxicities and increase the reproducibility of the immunotherapy's activity.

### Example 5: Isolation of CAR T-Cells Using Anti-PE MicroBeads

### 1.1 Principle of MACS® Separation

First, the CAR T-cells are contacted with a chimeric binding peptide labeled with an R-Phycoerythrin (PE) fluorescent moiety. Subsequently, the cells are contacted with Anti-PE MicroBeads UltraPure which binds the PE fluorescent moiety of the chimeric binding peptide which is bound to a CAR-expressing T-cell. Then the cell suspension is loaded on a MACS® Column which is placed in the magnetic field of a MACS Separator. The magnetically labeled CAR T-cells are retained in the column while the unlabeled cells run through. After removal of the column from the magnetic field, the magnetically retained cells can be eluted as the positively selected cell fraction.

### BACKGROUND INFORMATION

Anti-PE MicroBeads UltraPure have been especially developed for highly efficient separation of cells from debris-rich samples or other biological materials according to surface markers labeled with PE-conjugated primary antibodies, peptides, or ligands. After separation the PE-labeled cells can be directly detected by flow cytometry or fluorescence microscopy.

Fluorochrome tandem conjugates of R-Phycoerythrin and other fluorescent dyes that are often used in flow cytometry for third color analysis may also be recognized by Anti-PE MicroBeads UltraPure, e.g., PE-Cy™5, ECD, and PC5. For separation of cells labeled with primary antibodies conjugated to PE-Cy5, Anti-Cy5/Anti-Alexa Fluor® 647 MicroBeads are recommended.

### Protocol

Protocol should be performed quickly with cells kept cold and solutions pre-cooled. This will prevent capping of antibodies on the cell surface and a non-specific cell labeling.

Volumes for magnetic labeling described herein are for up to 10⁷ total cells. When working with fewer than 10⁷, use the same volumes as indicated. When working with higher cell numbers, scale up all reagent volumes and total volumes accordingly (e.g. for 2×10⁷ total cells, use twice the volume of all indicated reagent volumes and total volumes).

For optimal performance it is important to obtain a single cell suspension before magnetic separation. Pass cells through 30 μm nylon mesh (Pre-Separation Filters, 30 μm, #130-041-407) to remove cell clumps which may clog the column. Moisten filter with buffer before use.

The recommended incubation temperature is 2-8° C. Higher temperatures and/or longer incubation times may lead to non specific cell labeling. Working on ice may require increased incubation times.

The centrifugal force and centrifugation time mentioned below are recommendations. The optimal relative centrifugal force (RCF) and centrifugation time may be different depending on the cell sample.

Primary PE-conjugated antibodies should be titrated to determine the optimal staining dilution. Staining should not increase fluorescence intensity of the negative population.

### Magnetic Labeling

1. Determine cell number.
2. Centrifuge cell suspension at 300×g for 10 minutes. Aspirate supernatant completely.
3. Resuspend cell pellet and stain with the primary PE-conjugated antibody according to the manufacturer's recommendations.
4. Mix well and incubate for 10 minutes in the dark in the refrigerator (2-8° C.) or according to the manufacturer's recommendations.
5. Wash cells to remove unbound primary antibody by adding 1-2 mL of buffer per 10⁷ cells and centrifuge at 300×g for 10 minutes.
6. (Optional) Repeat washing step.
7. Aspirate supernatant completely and resuspend cell pellet in 80 μL of buffer per 107 total cells.
8. Add 20 μL of Anti-PE MicroBeads UltraPure per 10⁷ total cells.
9. Mix well and incubate for 15 minutes in the refrigerator (2-8° C.).
10. Wash cells by adding 1-2 mL of buffer per 10⁷ cells and centrifuge at 300×g for 10 minutes.
11. Aspirate supernatant completely.
12. Resuspend up to 10⁸ cells in 500 μL of buffer. Note: For higher cell numbers, scale up buffer volume accordingly. Note: For depletion with LD Columns, resuspend up to 1.25×10⁸ cells in 500 μL of buffer.
13. Proceed to magnetic separation.
   Magnetic separation with MS or LS Columns
1. Place column in the magnetic field of a suitable MACS Separator. For details refer to the respective MACS Column data sheet.
2. Prepare column by rinsing with the appropriate amount of buffer: MS: 500 μL LS: 3 mL
3. Apply cell suspension onto the column. Collect flow-through containing unlabeled cells.
4. Wash column with the appropriate amount of buffer. Collect unlabeled cells that pass through and combine with the flow through from step 3. MS: 3×500 μL LS: 3×3 mL
5. Remove column from the separator and place it on a suitable collection tube.
6. Pipette the appropriate amount of buffer onto the column. Immediately flush out the magnetically labeled cells by firmly pushing the plunger into the column. MS: 1 mL LS: 5 mL
7. (Optional) To increase the purity of the magnetically labeled cells, the eluted fraction can be enriched over a second MS or LS Column. Repeat the magnetic separation procedure as described in steps 1 to 6 by using a new column.
   Magnetic Separation with the autoMACS® Pro Separator
Refer to the respective user manual for instructions on how to use the autoMACS® Pro Separator. Buffers used for operating the autoMACS Pro Separator should have a temperature of ≥10° C.
1. Prepare and prime the instrument.
2. Apply tube containing the sample and provide tubes for collecting the labeled and unlabeled cell fractions. Place sample tube in row A of the tube rack and the fraction collection tubes in rows B and C.
3. For a standard separation choose one of the following programs:
1. Positive selection: Possel. Collect positive fraction in row C of the tube rack.
2. Depletion: Deplete. Collect negative fraction in row B of the tube rack.

### Informal Sequence Listing

SEQ ID NO: 1. ROR1 protein
QETELSVSAELVPTSSWNISSELNKDSYLTLDEPMNNITTSLGQTAELHCKVSGNPPPTIR
WFKNDAPVVQEPRRLSFRSTIYGSRLRIRNLDTTDTGYFQCVATNGKEVVSSTGVLFVK
FGPPPTASPGYSDEYEEDGFCQPYRGIACARFIGNRTVYMESLHMQGEIENQITAAFTMI
GTSSHLSDKCSQFAIPSLCHYAFPYCDETSSVPKPRDLCRDECEILENVLCQTEYIFARSN
PMILMRLKLPNCEDLPQPESPEAANCIRIGIPMADPINKNHKCYNSTGVDYRGTVSVTKS
GRQCQPWNSQYPHTHTFTALRFPELNGGHSYCRNPGNQKEAPWCFTLDENFKSDLCDIP
ACDSKDSKEKNKME
SEQ ID NO: 2. Linker
IEGRMD
SEQ ID NO: 3. Fc Tag
PKSSDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 4. ROR-Ig-Full length
QETELSVSAELVPTSSWNISSELNKDSYLTLDEPMNNITTSLGQTAELHCKVSGNPPPTIR
WFKNDAPVVQEPRRLSFRSTIYGSRLRIRNLDTTDTGYFQCVATNGKEVVSSTGVLFVK
FGPPPTASPGYSDEYEEDGFCQPYRGIACARFIGNRTVYMESLHMQGEIENQITAAFTMI
GTSSHLSDKCSQFAIPSLCHYAFPYCDETSSVPKPRDLCRDECEILENVLCQTEYIFARSN
PMILMRLKLPNCEDLPQPESPEAANCIRIGIPMADPINKNHKCYNSTGVDYRGTVSVTKS
GRQCQPWNSQYPHTHTFTALRFPELNGGHSYCRNPGNQKEAPWCFTLDENFKSDLCDIP
ACDSKDSKEKNKMEIEGRMDPKSSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEA
LHNHYTQKSLSLSPGK

### P EMBODIMENTS

P embodiment 1. A method of isolating a CAR T-cell, wherein said method comprises: (i) contacting a cell population comprising a CAR-expressing T-cell with a labeled CAR-binding peptide thereby forming a labeled CAR T-cell-peptide complex; (ii) contacting said labeled CAR T-cell-peptide complex with a label-binding magnetic particle thereby forming a bound labeled CAR T-cell-peptide complex; and (iii) separating said bound labeled CAR T-cell-peptide complex from said cell population, thereby isolating a CAR T-cell.

P embodiment 2. The method of P embodiment 1, wherein said bound labeled CAR T-cell peptide complex is isolated from said cell population with a magnet.

P embodiment 3. The method of P embodiment 2, wherein the CAR-T-cell is isolated from the labeled CAR-binding peptide and the label-binding magnetic particle, to obtain a purified CAR-T-cell.

### EMBODIMENTS

Embodiment 1. A method of isolating a chimeric antigen receptor (CAR)-expressing T-cell, said method comprising: (i) contacting a cell population comprising a CAR-expressing T-cell with a chimeric binding peptide comprising a CAR-binding domain, a ligand binding domain and a detectable moiety, thereby forming a chimeric peptide-CAR T-cell complex; (ii) contacting said chimeric peptide-CAR T-cell complex with a ligand, thereby forming a ligand-bound chimeric peptide-CAR T-cell complex; and (iii) separating said ligand-bound chimeric peptide-CAR T-cell complex from said cell population, thereby isolating a CAR T-cell population.

Embodiment 2. The method of embodiment 1, further comprising after said separating a step (iv) of expanding said isolated CAR T-cell population to form a purified CAR T-cell population.

Embodiment 3. The method of embodiment 1 or 2, wherein said isolated CAR T-cell population is expanded for about 3 days to about 60 days.

Embodiment 4. The method of embodiment 2 or 3, further comprising after said isolating a step (v) of administering a therapeutically effective amount of said purified CAR T-cell population to a subject in need thereof.

Embodiment 5. The method of embodiment 4, wherein said subject is a human.

Embodiment 6. The method of embodiment 4 or 5, wherein said subject is a cancer subject.

Embodiment 7. The method of any one of embodiments 4-6, wherein said cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer.

Embodiment 8. The method of any one of embodiments 1-7, wherein said separating comprises contacting said ligand-bound chimeric peptide-CAR T-cell complex with a magnetic particle or a solid support.

Embodiment 9. The method of any one of embodiments 1-8, wherein said chimeric peptide-CAR T-cell complex is formed by binding of said CAR-binding domain to said CAR of said CAR-expressing T-cell.

Embodiment 10. The method of any one of embodiments 1-9, wherein said ligand-bound chimeric peptide-CAR T-cell complex is formed by binding of said detectable moiety to said ligand.

Embodiment 11. The method of any one of embodiments 1-9, wherein said ligand-bound chimeric peptide-CAR T-cell complex is formed by binding of said ligand binding domain to said ligand.

Embodiment 12. The method of any one of embodiments 1-11, wherein said binding is covalent or non-covalent.

Embodiment 13. The method of any one of embodiments 1-12, wherein said ligand is a protein, a nucleic acid, or a small molecule.

Embodiment 14. The method of any one of embodiments 1-13, wherein said ligand is an antibody domain.

Embodiment 15. The method of any one of embodiments 1-14, wherein said ligand is bound to a metal-coated particle or a solid support.

Embodiment 16. The method of any of embodiments 1-15, wherein said CAR-expressing T-cell is a ROR1 CAR T-cell, a ROR2 CAR T-cell, or a CD19 CAR T-cell.

Embodiment 17. The method of any of embodiments 1-16, wherein said CAR-binding domain and said ligand binding domain are connected through a chemical linker.

Embodiment 18. The method of embodiment 17, wherein said chemical linker is a peptide.

Embodiment 19. The method of embodiment 17 or 18, wherein said chemical linker comprises an amino acid sequence of about 2 to about 25 amino acids in length.

Embodiment 20. The method of any one of embodiments 17-19, wherein said chemical linker comprises the amino acid sequence of SEQ ID NO:2.

Embodiment 21. The method of any one of embodiments 1-20, wherein said chimeric binding peptide comprises an amino acid sequence of about 25 to about 1000 amino acids in length.

Embodiment 22. The method of any one of embodiments 1-21, wherein said CAR-binding domain comprises an amino acid sequence of about 25 to about 500 amino acids in length.

Embodiment 23. The method of any one of embodiments 1-22, wherein said CAR-binding domain is a cancer antigen or fragment thereof.

Embodiment 24. The method of any one of embodiments 1-23, wherein said CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme.

Embodiment 25. The method of any one of embodiments 1-24, wherein said CAR binding domain is a ROR domain.

Embodiment 26. The method of any one of embodiments 1-25, wherein said CAR-binding domain is a ROR1 domain.

Embodiment 27. The method of any one of embodiments 1-26, wherein said CAR-binding domain is a ROR1 Ig-like domain.

Embodiment 28. The method of any one of embodiments 1-27, wherein said CAR-binding domain comprises the amino acid sequence of SEQ ID NO:1.

Embodiment 29. The method of any one of embodiments 1-28, wherein said ligand binding domain comprises an amino acid sequence of about 1 to about 500 amino acids in length.

Embodiment 30. The method of any one of embodiments 1-29, wherein said ligand binding domain is an antibody domain.

Embodiment 31. The method of any one of embodiments 1-30, wherein said ligand binding domain is an Fc domain.

Embodiment 32. The method of any one of embodiments 1-31, wherein said ligand binding domain comprises the amino acid sequence of SEQ ID NO:3.

Embodiment 33. The method of any one of embodiments 1-32, wherein said detectable moiety is a fluorescent moiety.

Embodiment 34. The method of any one of embodiments 1-33, wherein said detectable moiety is a phycoerythrin (PE) moiety.

Embodiment 35. The method of any one of embodiments 1-34, wherein said chimeric binding peptide comprises a ROR1 domain, an Fc domain and one or more PE moieties.

Embodiment 36. The method of any one of embodiments 1-35, wherein said chimeric binding peptide comprises the amino acid sequence of SEQ ID NO:4.

Embodiment 37. The method of any one of embodiments 1-36, wherein said chimeric binding peptide comprises the sequence of SEQ ID NO:4.

Embodiment 38. A chimeric peptide-chimeric antigen receptor (CAR) T-cell complex comprising: a CAR-expressing T-cell bound to a chimeric binding peptide, said chimeric binding peptide comprising a CAR-binding domain a ligand binding domain, and a detectable moiety.

Embodiment 39. The complex of embodiment 38, wherein said CAR-binding domain is bound to said CAR.

Embodiment 40. The complex of embodiment 38 or 39, wherein said CAR-expressing T-cell is a ROR CAR T-cell, a ROR2 CAR T-cell, or a CD19 CAR T-cell.

Embodiment 41. The complex of any one of embodiments 38-40, wherein said complex further comprises a ligand bound to said ligand binding domain.

Embodiment 42. The complex of any one of embodiments 38-41, wherein said binding is covalent or non-covalent.

Embodiment 43. The complex of any one of embodiments 38-42, wherein said ligand is a protein, a nucleic acid or a small molecule.

Embodiment 44. The complex of any one of embodiments 38-41, wherein said ligand is an antibody domain.

Embodiment 45. The complex of any one of claims 38-44, wherein said complex is bound to a metal-coated particle or a solid support through said ligand.

Embodiment 46. The complex of any one of embodiments 38-43, wherein said CAR-binding domain and said ligand binding domain are connected through a chemical linker.

Embodiment 47. The complex of embodiment 46, wherein said chemical linker is a peptide.

Embodiment 48. The complex of embodiment 46 or 47, wherein said chemical linker comprises an amino acid sequence of about 2 about 25 amino acids in length.

Embodiment 49. The complex of any one of embodiments 46-48, wherein said chemical linker comprises the amino acid sequence of SEQ ID NO:2.

Embodiment 50. The complex of any one of embodiments 38-49, wherein said chimeric binding peptide comprises an amino acid sequence of about 25 to about 1000 amino acids in length.

Embodiment 51. The complex of any one of embodiments 38-50, wherein said CAR-binding domain comprises an amino acid sequence of about 25 to about 500 amino acids in length.

Embodiment 52. The complex of any one of embodiments 38-51, wherein said CAR-binding domain is a cancer antigen or fragment thereof.

Embodiment 53. The complex of any one of embodiments 38-52, wherein said CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme.

Embodiment 54. The complex of any one of embodiments 38-53, wherein said CAR-binding domain is a ROR domain.

Embodiment 55. The complex of any one of embodiments 38-54, wherein said CAR-binding domain is a ROR1 domain.

Embodiment 56. The complex of any one of embodiments 38-55, wherein said CAR-binding domain is a ROR1 Ig-like domain.

Embodiment 57. The complex of any one of embodiments 38-56, wherein said CAR-binding domain comprises the sequence of SEQ ID NO:1.

Embodiment 58. The complex of any one of embodiments 38-57, wherein said ligand binding domain comprises an amino acid sequence of about 1 to about 500 amino acids in length.

Embodiment 59. The complex of any one of embodiments 38-58, wherein said ligand binding domain is an antibody domain.

Embodiment 60. The complex of any one of embodiments 38-59, wherein said ligand binding domain is an Fc domain.

Embodiment 61. The complex of any one of embodiments 38-60, wherein said ligand binding domain comprises the sequence of SEQ ID NO:3.

Embodiment 62. The complex of any one of embodiments 38-61, wherein said detectable moiety is a fluorescent moiety.

Embodiment 63. The complex of any one of embodiments 38-62, wherein said detectable moiety is a phycoerythrin (PE) moiety.

Embodiment 64. The complex of any one of embodiments 38-63, wherein said chimeric binding peptide comprises a ROR1 domain, an Fc domain and one or more PE moieties.

Embodiment 65. The complex of any one of embodiments 38-64, wherein said chimeric binding peptide comprises the sequence of SEQ ID NO:4.

Embodiment 66. The complex of any one of embodiments 38-65, wherein said complex comprises a ROR1 CAR T-cell, a ROR1 domain, an Fc domain and one or more PE moieties.

Embodiment 67. A pharmaceutical composition comprising a therapeutically effective amount of said purified CAR T-cell population of embodiment 2 and a pharmaceutically acceptable excipient.

Embodiment 68. A method of treating cancer in a subject in need thereof comprising administering to said subject a therapeutically effective amount of said purified CAR T-cell of embodiment 2 or the pharmaceutical composition of embodiment 67, thereby treating said cancer.

Embodiment 69. The method of embodiment 68, wherein said cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer.

What is claimed is:

1. A method of isolating a chimeric antigen receptor (CAR)-expressing T-cell, said method comprising:
(i) contacting a cell population comprising a CAR-expressing T-cell with a chimeric binding peptide comprising a CAR-binding domain, a ligand binding domain and a detectable moiety, thereby forming a chimeric peptide-CAR T-cell complex;
(ii) contacting said chimeric peptide-CAR T-cell complex with a ligand, thereby forming a ligand-bound chimeric peptide-CAR T-cell complex; and
(iii) separating said ligand-bound chimeric peptide-CAR T-cell complex from said cell population, thereby isolating a CAR T-cell population.

2. The method of claim 1, further comprising after said separating a step (iv) of expanding said isolated CAR T-cell population to form a purified CAR T-cell population.

3. The method of claim 1, wherein said isolated CAR T-cell population is expanded for about 3 days to about 60 days.

4. The method of claim 2, further comprising after said isolating a step (v) of administering a therapeutically effective amount of said purified CAR T-cell population to a subject in need thereof.

5. The method of claim 4, wherein said subject is a human.

6. The method of claim 4, wherein said subject is a cancer subject.

7. The method of claim 4, wherein said cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer.

8. The method of claim 1, wherein said separating comprises contacting said ligand-bound chimeric peptide-CAR T-cell complex with a magnetic particle or a solid support.

9. The method of claim 1, wherein said chimeric peptide-CAR T-cell complex is formed by binding of said CAR-binding domain to said CAR of said CAR-expressing T-cell.

10. The method of claim 1, wherein said ligand-bound chimeric peptide-CAR T-cell complex is formed by binding of said detectable moiety to said ligand.

11. The method of claim 1, wherein said ligand-bound chimeric peptide-CAR T-cell complex is formed by binding of said ligand binding domain to said ligand.

12. The method of claim 1, wherein said binding is covalent or non-covalent.

13. The method of claim 1, wherein said ligand is a protein, a nucleic acid, or a small molecule.

14. The method of claim 1, wherein said ligand is an antibody domain.

15. The method of claim 1, wherein said ligand is bound to a metal-coated particle or a solid support.

16. The method of claim 1, wherein said CAR-expressing T-cell is a ROR1 CAR T-cell, a ROR2 CAR T-cell, or a CD19 CAR T-cell.

17. The method of claim 1, wherein said CAR-binding domain and said ligand binding domain are connected through a chemical linker.

18. The method of claim 17, wherein said chemical linker is a peptide.

19. The method of claim 17, wherein said chemical linker comprises an amino acid sequence of about 2 to about 25 amino acids in length.

20. The method of claim 17, wherein said chemical linker comprises the amino acid sequence of SEQ ID NO:2.

21. The method of claim 1, wherein said chimeric binding peptide comprises an amino acid sequence of about 25 to about 1000 amino acids in length.

22. The method of claim 1, wherein said CAR-binding domain comprises an amino acid sequence of about 25 to about 500 amino acids in length.

23. The method of claim 1, wherein said CAR-binding domain is a cancer antigen or fragment thereof.

24. The method of claim 1, wherein said CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme.

25. The method of claim 1, wherein said CAR binding domain is a ROR domain.

26. The method of a claim 1, wherein said CAR-binding domain is a ROR1 domain.

27. The method of claim 1, wherein said CAR-binding domain is a ROR1 Ig-like domain.

28. The method of claim 1, wherein said CAR-binding domain comprises the amino acid sequence of SEQ ID NO:1.

29. The method of claim 1, wherein said ligand binding domain comprises an amino acid sequence of about 1 to about 500 amino acids in length.

30. The method of claim 1, wherein said ligand binding domain is an antibody domain.

31. The method of claim 1, wherein said ligand binding domain is an Fc domain.

32. The method of 1, wherein said ligand binding domain comprises the amino acid sequence of SEQ ID NO:3.

33. The method of claim 1, wherein said detectable moiety is a fluorescent moiety.

34. The method of claim 1, wherein said detectable moiety is a phycoerythrin (PE) moiety.

35. The method of claim 1, wherein said chimeric binding peptide comprises a ROR1 domain, an Fc domain and one or more PE moieties.

36. The method of claim 1, wherein said chimeric binding peptide comprises the amino acid sequence of SEQ ID NO:4.

37. A chimeric peptide-chimeric antigen receptor (CAR) T-cell complex comprising: a CAR-expressing T-cell bound to a chimeric binding peptide, said chimeric binding peptide comprising a CAR-binding domain a ligand binding domain, and a detectable moiety.

38. The complex of claim 37, wherein said CAR-binding domain is bound to said CAR.

39. The complex of claim 37, wherein said CAR-expressing T-cell is a ROR CAR T-cell, a ROR2 CAR T-cell, or a CD19 CAR T-cell.

40. The complex of claim 37, wherein said complex further comprises a ligand bound to said ligand binding domain.

41. The complex of claim 37, wherein said binding is covalent or non-covalent.

42. The complex of claim 37, wherein said ligand is a protein, a nucleic acid or a small molecule.

43. The complex of claim 37, wherein said ligand is an antibody domain.

44. The complex of claim 37, wherein said complex is bound to a metal-coated particle or a solid support through said ligand.

45. The complex of claim 37, wherein said CAR-binding domain and said ligand binding domain are connected through a chemical linker.

46. The complex of claim 45, wherein said chemical linker is a peptide.

47. The complex of claim 45, wherein said chemical linker comprises an amino acid sequence of about 2 about 25 amino acids in length.

48. The complex of claim 45, wherein said chemical linker comprises the amino acid sequence of SEQ ID NO:2.

49. The complex of claim 37, wherein said chimeric binding peptide comprises an amino acid sequence of about 25 to about 1000 amino acids in length.

50. The complex of claim 37, wherein said CAR-binding domain comprises an amino acid sequence of about 25 to about 500 amino acids in length.

51. The complex of claim 37, wherein said CAR-binding domain is a cancer antigen or fragment thereof.

52. The complex of claim 37, wherein said CAR-binding domain is a receptor tyrosine kinase domain, a tumor necrosis factor (TNF) receptor, an interleukin cytokine, an interleukin receptor, a non-classical serine protease, a B cell surface molecule, a T-cell surface molecule, a natural killer (NK) cell surface molecule, a mucin, a cell adhesion molecule, a disialoganglioside, a C-type lectin-like receptor, a tight junction protein, a Lewis antigen, or a zinc metalloenzyme.

53. The complex of claim 37, wherein said CAR-binding domain is a ROR domain.

54. The complex of claim 37, wherein said CAR-binding domain is a ROR1 domain.

55. The complex of claim 37, wherein said CAR-binding domain is a ROR1 Ig-like domain.

56. The complex of claim 37, wherein said CAR-binding domain comprises the sequence of SEQ ID NO:1.

57. The complex of claim 37, wherein said ligand binding domain comprises an amino acid sequence of about 1 to about 500 amino acids in length.

58. The complex of claim 37, wherein said ligand binding domain is an antibody domain.

59. The complex of claim 37, wherein said ligand binding domain is an Fc domain.

60. The complex of claim 37, wherein said ligand binding domain comprises the sequence of SEQ ID NO:3.

61. The complex of claim 37, wherein said detectable moiety is a fluorescent moiety.

62. The complex of claim 37, wherein said detectable moiety is a phycoerythrin (PE) moiety.

63. The complex of claim 37, wherein said chimeric binding peptide comprises a ROR1 domain, an Fc domain and one or more PE moieties.

64. The complex of claim 37, wherein said chimeric binding peptide comprises the sequence of SEQ ID NO:4.

65. The complex of claim 37, wherein said complex comprises a ROR1 CAR T-cell, a ROR1 domain, an Fc domain and one or more PE moieties.

66. A pharmaceutical composition comprising a therapeutically effective amount of said purified CAR T-cell population of claim 2 and a pharmaceutically acceptable excipient.

67. A method of treating cancer in a subject in need thereof comprising administering to said subject a therapeutically effective amount of said purified CAR T-cell of claim 2 or the pharmaceutical composition of claim 66, thereby treating said cancer.

68. The method of claim 67, wherein said cancer is lymphoma, chronic lymphocytic leukemia (CLL), small lymphocytic lymphoma, marginal cell B-Cell lymphoma, Burkett's Lymphoma, leukemia, chronic B cell leukemia, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, or head and neck cancer.


SEQUENCE LISTING (4 sequences)

SEQ ID NO: 1 - protein, 374 aa, synthetic construct
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKME

SEQ ID NO: 2 - protein, 6 aa, synthetic construct
IEGRMD

SEQ ID NO: 3 - protein, 231 aa, synthetic construct
PKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 611 aa, synthetic construct
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK